(19) United States
(12) Patent Application Publication
(10) Pub. No.: US 2025/0170066 A1
(43) Pub. Date: May 29, 2025

(54) TREATMENT OF SOLID TUMORS WITH NEGATIVELY CHARGED PARTICLES

(71) Applicant: ONCOUR PHARMA, INC., Skokie, IL (US)

(72) Inventors: John Puisis, Northbrook, IL (US); Adam Elhofy, Chicago, IL (US); Michael Boyne, Skokie, IL (US); Joseph Podojil, Skokie, IL (US); Tushar Murthy, Northbrook, IL (US)

(21) Appl. No.: 18/840,232

(22) PCT Filed: Feb. 27, 2023
(86) PCT No.: PCT/US2023/063367

§ 371 Date: Aug. 21, 2024

Related U.S. Application Data
(60) Provisional application No. 63/313,959, filed on Feb. 25, 2022.

(51) Int. Cl. A61K 9/16; A61K 47/12; A61K 47/26; A61P 35/00

(52) U.S. Cl. CPC A61K 9/1647; A61K 47/12; A61K 47/26; A61P 35/00

(57) ABSTRACT

The present disclosure generally relates to the methods of treating solid tumors using negatively charged particles.

### CROSS-REFERENCE TO RELATED APPLICATIONS

This application claims priority to and the benefit of U.S. Provisional Patent Application No. 63/313,959 filed on Feb. 25, 2022, which is incorporated by reference in its entirety for all purposes.

### FIELD OF THE DISCLOSURE

The present disclosure generally relates to the methods of treating solid tumors using negatively charged particles.

### BACKGROUND

Solid tumors are one of the leading causes of mortality in the US and worldwide. Despite aggressive therapeutic interventions such as chemotherapies and immunotherapies, solid tumors account for nearly 500,000 annual deaths in the US and 9 million annual deaths worldwide. The current standard of care for solid tumors relies on multiple lines of treatment involving surgical tumor resection (when possible), radiation therapy, and monotherapy or combination therapy with cytotoxic agents, targeted therapies, and immunotherapies. A significant number of patients cannot tolerate these intensive treatment regimens due to severe side-effects (e.g., hepatotoxicity, cytopenia, gastrointestinal toxicity, and neurotoxicity). Of the patients who do receive these treatment regimens, a large number do not respond, develop treatment resistance after an initial response, or experience disease recurrence within a short period of time.

The immune system plays a critical role in influencing disease progression, response to treatment, and treatment outcomes in solid tumors. Immune suppression due to dysregulation of myeloid derived cells such as tumor-associated macrophages (tams) and myeloid derived suppressor cells (mdscs) during disease progression contributes to poor response to treatment, treatment resistance, and risk of recurrence. These dysregulated myeloid derived cells engage in pro-tumor functions such as remodeling of the tumor microenvironment promoting immune exclusion, and contributing to immune exhaustion. Together, these activities promote tumor growth, metastasis, and mortality despite aggressive treatment. There is an urgent need for therapies that can overcome and/or reverse this immune suppression and thus enable re activation of the endogenous anti-tumor immune function to improve patient outcomes.

### SUMMARY

Negatively charged particles made from biodegradable materials and free from attached (covalent and non-covalent), adhered, or embedded drugs (e.g., peptides, antigenic moieties, and bioactive materials) are designed to have immunomodulatory properties, due at least in part to the particle diameter (size), surface charge (zeta potential), and size distribution. These negatively charged particles are free of antigens or therapeutics. The physicochemical properties of negatively charged particles are designed for targeted uptake by myeloid-derived cells (e.g., monocytes, macrophages, and neutrophils). This targeted uptake results in downstream immunomodulation via induction of cell-surface IL-15 expression on leukocytes, activation of anti-tumor T cells and NK cells, and production of anti-tumor cytokines/chemokines (e.g., TNF-α, MIP-1β, and RANTES (CCL5)). The immunomodulatory properties of negatively charged particles relieve immune suppression and re-activate the endogenous anti-tumor immune function. In multiple preclinical solid tumor models representing treatment responsive, treatment resistant, and metastatic tumors, negatively charged particles have demonstrated efficacy at inhibiting tumor growth and metastasis leading to improved survival. Thus, anti-tumor mechanism of action is highly relevant for the treatment of solid tumors.

Provided herein is a method of treating solid tumors in a subject comprising administering to the subject negatively charged particles, wherein the negatively charged particles are administered at a dose of about 0.1 to 15 mg/kg. Also provided herein is a method of inducing an anti-tumor immune response in a subject suffering from solid tumors, the method comprising administering to the subject negatively charged particles, wherein the negatively charged particles are administered at a dose of about 0.1 mg/kg to about 15 mg/kg. In various embodiments, the negatively charged particles are administered at a dose of about 0.5 mg/kg to about 10 mg/kg, from about 1 mg/kg to about 8 mg/kg, from about 1.5 mg/kg to about 15 mg/kg, from about 2 mg/kg to about 15 mg/kg, from about 2 mg/kg to about 10 mg/kg, from about 3 mg/kg to about 10 mg/kg, from about 4 mg/kg to about 10 mg/kg, from about 4 mg/kg to about 15 mg/kg, from about 5 mg/kg to about 15 mg/kg, or from about 2 mg/kg to about 8 mg/kg. In various embodiments, negatively charged particles are administered at a dose of about 0.1 mg/kg, 0.25 mg/kg, 0.5 mg/kg, 1.0 mg/kg, 2.0 mg/kg, 3.0 mg/kg, 4.0 mg/kg, 5 mg/kg, 6 mg/kg, 8.0 mg/kg, 10 mg/kg, 12 mg/kg, or 15 mg/kg. In various embodiments, negatively charged particles are administered at a dose of about 10 mg, 25 mg, 50 mg, 75 mg, 100 mg, 125 mg, 150 mg, 175 mg, 200 mg, 225 mg, 250 mg, 275 mg, 300 mg, 325 mg, 350 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg, 800 mg, 850 mg, 875 mg, 900 mg, 950 mg, 975 mg, or 1000 mg.

In various embodiments, the negatively charged particles are biodegradable. In embodiments, the negatively charged particles are polyglycolic acid (PGA) particles, polylactic acid (PLA) particles, poly (lactic-co-glycolic acid) (PLGA) particles, polystyrene particles, diamond particles, or iron, zinc, cadmium, gold, or silver particles, or combinations thereof.

In some embodiments, the negatively charged particles are poly (lactic-co-glycolic acid) (PLGA) particles. In various embodiments, the particles comprise PLGA at a co-polymer ratio of about 100:0 to about 0:100, about 90:10 to about 10:90, about 80:20 to about 20:80, or about 50:50 polyglycolic acid:polylactic acid. In various embodiments, the particle comprises 50:50 polylactic acid:polyglycolic acid. In various embodiments, the particle comprises polylactic acid:polyglycolic acid from about 99:1 to about 1:99, e.g., about 99:1, about 95:5, about 90:10, about 85:15, about 80:20, about 75:25, about 70:30, about 65:35, about 60:40, about 55:45, about 50:50, about 45:55, about 40:60, about 35:65, about 30:70, about 25:75, about 20:80, about 15:85, about 10:90, about 5:95, and about 1:99, including all values and ranges that lie in between these values.

In various embodiments, negatively charged particles are surface functionalized by carboxylation. In various embodiments, negatively charged particles have a negative zeta potential. In some embodiments, the carboxylation produces a negative charge on the particles. In some embodiments, the carboxylation increases the negative charge of the negatively charged particles. In various embodiments, the negative zeta potential of negatively charged particles is about −100 mV to about 0 mV. In various embodiments, the zeta potential of the particles is from about −100 mV to about −25 mV, from about −100 mV to about −30 mV, from about −80 mV to about −30 mV, from about −75 mV to about −30 mV, from about −70 mV to about −30 mV, from about −75 to about −35 mV, from about −70 mV to about −25 mV, from about −60 mV to about −30 mV, from about −60 mV to about −35 mV, or from about −50 mV to about −30 mV. In various embodiments, the zeta potential is about −25 mV, −30 mV, −35 mV, −40 mV, −45 mV, −50 mV, −55 mV, −60 mV, −65 mV, −70 mV, −75 mV, −80 mV, −85 mV, −90 mV, −95 mV or −100 mV, including all values and ranges therein. In various embodiments, the negatively charged particles have a negative zeta potential of about −30 mV to −60 mV.

In various embodiments, the size, or diameter, of negatively charged particles is from about 0.05 μm to about 10 μm. In various embodiments, the diameter of negatively charged particles is from about 0.1 μm to about 10 μm. In various embodiments, the diameter of negatively charged particles is from about 0.1 μm to about 5 μm. In various embodiments, the diameter of negatively charged particles is from 0.1 μm to about 3 μm. In various embodiments, the diameter of negatively charged particles is from 0.3 μm to about 5 μm. In various embodiments, the diameter of negatively charged particles is from 0.3 μm to about 3 μm. In various embodiments, the diameter of negatively charged particles is from about 0.3 μm to about 1 μm. In various embodiments, the diameter of negatively charged particles is from about 0.4 μm to about 1 μm. In various embodiments, the negatively charged particles have a diameter of about 100 nm to 10000 nm, about 100 nm to 5000 nm, about 100 nm to 3000 nm, about 100 nm to 2000 nm, about 100 nm to 1500 nm, about 300 nm to 5000 nm, about 300 nm to 3000 nm, about 300 nm to 1000 nm, about 300 nm to 800 nm, about 400 nm to 800 nm, or about 200 nm to 700 nm. In various embodiments, the negatively charged particles have a diameter of about 50 nm, 100 nm, 200 nm, 300 nm, 400 nm, 500 nm, 600 nm, 700 nm, 800 nm, 900 nm, 1000 nm, 1100 nm, 1200 nm, 1300 nm, 1400 nm, 1500 nm, or 2000 nm, including all values and ranges therein. In various embodiments, the diameter of the negatively charged particles is from 350 nm to 800 nm.

In various embodiments, the negatively charged particles have a homogenous size distribution. In various embodiments, the negatively charged particles have a homogenous size distribution wherein at least 90% of the particles have a diameter of about 0.05 μm to about 10 μm, about 0.1 μm to about 10 μm, about 0.1 μm to about 5 μm, about 0.1 μm to about 3 μm, about 0.3 μm to about 5 μm, about 0.3 μm to about 3 μm, including all values and ranges therein. In various embodiments, the negatively charged particles have a homogenous size distribution wherein at least 90% of the particles have a diameter of about 100 nm to 10000 nm, about 100 nm to 5000 nm, about 100 nm to 3000 nm, about 100 nm to 2000 nm, about 300 nm to 5000 nm, about 300 nm to 3000 nm, about 300 nm to 1000 nm, about 300 nm to 800 nm, about 400 nm to 800 nm, or about 200 nm to 700 nm, including all values and ranges therein. In various embodiments, the negatively charged particles have a diameter of about 50 nm, 100 nm, 200 nm, 300 nm, 400 nm, 500 nm, 600 nm, 700 nm, 800 nm, 900 nm, 1000 nm, 1100 nm, 1200 nm, 1300 nm, 1400 nm, 1500 nm, or 2000 nm including all values and ranges therein. In various embodiments, the negatively charged particles have a homogenous size distribution wherein at least 50% of the particles have a diameter of about 0.05 μm to about 10 μm, about 0.1 μm to about 10 μm, about 0.1 μm to about 5 μm, about 0.1 μm to about 3 μm, about 0.3 μm to about 5 μm, and about 0.3 μm to about 3 μm, including all values and ranges therein. In various embodiments, the particles have a homogenous size distribution wherein at least 50% of the particles have a diameter of about 100 nm to about 10000 nm, about 100 nm to about 5000 nm, about 100 nm to about 3000 nm, about 100 nm to about 2000 nm, about 300 nm to about 5000 nm, about 300 nm to about 3000 nm, about 300 nm to about 1000 nm, about 300 nm to about 800 nm, about 400 nm to about 800 nm, or about 200 nm to about 700 nm, including all values and ranges therein. In various embodiments, the negatively charged particles have a diameter of about 50 nm, 100 nm, 200 nm, 300 nm, 400 nm, 500 nm, 600 nm, 700 nm, 800 nm, 900 nm, 1000 nm, 1100 nm, 1200 nm, 1300 nm, 1400 nm, 1500 nm, or 2000 nm, including all values and ranges therein.

In various embodiments, the negatively charged particles have a homogenous size distribution wherein at least 10% of the particles have a diameter of about 0.05 μm to about 10 μm, about 0.1 μm to about 10 μm, about 0.1 μm to about 5 μm, about 0.1 μm to about 3 μm, about 0.3 μm to about 5 μm, or about 0.3 μm to about 3 μm, including all values and ranges therein. In various embodiments, the negatively charged particles have a homogenous size distribution wherein at least 10% of the particles have a diameter of about 100 nm to 10000 nm, about 100 nm to 5000 nm, about 100 nm to 3000 nm, about 100 nm to 2000 nm, about 300 nm to 5000 nm, about 300 nm to 3000 nm, about 300 nm to 1000 nm, about 300 nm to 800 nm, about 400 nm to 800 nm, or about 200 nm to 700 nm, including all values and ranges therein. In various embodiments, the negatively charged particles have a diameter of about 50 nm, 100 nm, 200 nm, 300 nm, 400 nm, 500 nm, 600 nm, 700 nm, 800 nm, 900 nm, 1000 nm, 1100 nm, 1200 nm, 1300 nm, 1400 nm, 1500 nm, or 2000 nm, including all values and ranges therein.

In various embodiments, the negatively charged particles are PLGA particles having a zeta potential ranging from about −80 mV to about −30 mV and a diameter ranging from about 200 nm to about 2000 nm.

In various embodiments, negatively charged particles are administered intravenously, subcutaneously, intramuscularly, intraperitoneally, intranasally, transdermally, ocularly or orally.

In various embodiments, negatively charged particles are administered at a concentration of about 0.05 mg/mL to about 50 mg/mL. In various embodiments, negatively charged particles are administered at a concentration of about 0.1 mg/mL, 0.5 mg/mL, 1 mg/mL, 2 mg/mL, 3 mg/mL, 4 mg/mL, 5 mg/mL, 6 mg/mL, 7 mg/mL, 8 mg/mL, 9 mg/mL, 10 mg/mL, 11 mg/mL, 12.5 mg/mL, 15 mg/mL, 17.5 mg/mL, 20 mg/mL, 25 mg/mL, 30 mg/mL, 40 mg/mL, or 50 mg/mL including all values and ranges therein. In various embodiments, negatively charged particles are administered via intravenous infusion lasting about 1, 2, 3, 4, 5, 6, 7, or 8 hours including all values and ranges therein.

In various embodiments, the negatively charged particles are administered once daily, twice daily, three times per day, seven times per week, six times per week, five times per week, four times per week, three times per week, twice weekly, once weekly, once every two weeks, once every three weeks, once every 4 weeks, once every two months, once every three months, once every 6 months or once per year. In various embodiments, the negatively charged surface functionalized particle and/or the cancer therapeutic is administered for 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, or 52 weeks, or more including all values and ranges therein.

In various embodiments, the negatively charged particles are administered in treatment cycles. In various embodiments, one treatment cycle consists of 1, 2, 3, 4, 5, 6, or 7 doses is administered weekly. In various embodiments, the subject is administered 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, or 52 treatment cycles of negatively charged particles.

In various embodiments, the subject has a cancer selected from the group consisting of brain cancer, skin cancer, eye cancer, breast cancer, prostate cancer, lung cancer, esophageal cancer, head and neck cancer, cervical cancer, liver cancer, colon cancer, bone cancer, uterine cancer, ovarian cancer, bladder cancer, stomach cancer, oral cancer, thyroid cancer, kidney cancer, and testicular cancer.

In various embodiments, administering negatively charged particles to a subject in need thereof improves one or more symptoms associated with the solid tumor(s) or cancer. In various embodiments, the one or more symptoms are selected from the group consisting of tumor size, tumor burden, and tumor metastasis. In various embodiments, administering negatively charged particles to the subject improves survival.

In various embodiments, the efficacy of negatively charged particles at improving one or more symptoms of the cancer in a subject is determined from the assay of one or more biological samples from the subject. In various embodiments, the biological samples are selected from the group consisting whole-blood, peripheral blood, peripheral blood mononuclear cells (PBMCs), serum, plasma, urine, cerebrospinal fluid (CSF), stool, a tissue biopsy, and a bone-marrow biopsy.

In various embodiments, the negatively charged particles are administered to a subject in need thereof alone or in combination with one or more additional cancer therapeutics. In various embodiments, the negatively charged particles are administered prior to, in combination with, or after the administration of one or more additional cancer therapeutics. In various embodiments, the negatively charged particles are administered as a neoadjuvant prior to surgical resection of the solid tumor. In various embodiments, the negatively charged particles are administered as an adjuvant following surgical resection of the solid tumor.

In various embodiments, the cancer therapeutic is a chemotherapeutic selected from the group consisting of growth inhibitors, DNA-replication inhibitors, kinase inhibitors, signaling cascade inhibitors, angiogenesis inhibitors, metabolic inhibitors, amino acid synthesis inhibitors, selective inhibitors of oncogenic proteins, inhibitors of metastasis, inhibitors of anti-apoptosis factors, apoptosis inducers, nucleoside signaling inhibitors, enzyme inhibitors, proteasome inhibitors, and DNA-damaging agents.

In various embodiments, the cancer therapeutic comprises one or more biologic agents selected from the group consisting of cytokines, angiogenesis inhibitors, immune checkpoint modulators, enzymes, and monoclonal antibodies.

In various embodiments, cytokines are selected from the group consisting of transforming growth factors, tumor necrosis factor, interferons, and interleukins. Exemplary cytokines include, but are not limited to, IFN-alpha, IFN-beta, IFN-gamma, IL-1, IL-2, IL-4, IL-5, IL-6, IL-7, IL-10, IL-11, IL-12, IL-13, IL-15, IL-17, IL-18, IL-21, members of the transforming growth factor beta superfamily, including TGF-β1, TGF-β2 and TGF-β3, tumor necrosis factor alpha, Granulocyte colony-stimulating factor (G-CSF), and Granulocyte macrophage colony-stimulating factor (GM-CSF).

In various embodiments, the cancer therapeutic comprises an enzyme. In various embodiments, the cancer therapeutic comprises an enzyme that targets T-cells, B-cells, APCs, monocytes, MDSCs, TAMs, neutrophils, other monocyte-derived cells, tumor-associated stroma, cancer stem cells, mesenchymal stem cells, extracellular matrix, and amino acids. In various embodiments, the cancer therapeutic comprises an enzyme selected from the group comprising asparaginase, kynurininase, L-arginine deiminase, L-methionine-γ-lyase, one or more amino acid degrading enzymes, and one or more nucleoside degrading enzymes.

In various embodiments the monoclonal antibodies are mono-specific, bi-specific, tri-specific, or bispecific T-cell engaging (BiTE) antibodies.

In various embodiments, monoclonal antibodies are selected from the group comprising Alemtuzumab, Bevacizumab, Brentuximab, Cetuximab, Denosumab, Ibritumomab, Trastuzumab, Panitumumab, Pertuzumab, and Rituximab. In various embodiments, monoclonal antibodies target receptor tyrosine kinase, EGFR, VEGF, VEGFR, PDGF, PDGFR, TGF-β, TGF-β-LAP, SIRP-α, CD47, CD39, CD73, or fibroblast activation protein (FAP). In various embodiments, the immune checkpoint modulators target Programmed cell death protein 1 (PD1), Programmed cell death protein ligand-1 (PD-L1), cytotoxic T-lymphocyte-associated protein 4 (CTLA-4), T-cell Immunoglobulin and mucin-domain containing-3 (TIM-3), Lymphocyte-activation Gene 3 (LAG-3) and/or TIGIT (T cell immunoreceptor with Ig and ITIM domains). In various embodiments, the immune checkpoint modulator is an antibody selected from the group consisting of ipilimumab, tremelimumab, pembrolizumab, nivolumab, atezolizumab, avelumab, Cemiplimab, and durvalumab. In various embodiments, the cancer therapeutic comprises one or more cell-based therapies selected from the group consisting of adoptive cell transfer, tumor-infiltrating leukocyte therapy, chimeric antigen receptor T-cell (CAR-T), NK-cell therapy and stem cell therapy.

In various embodiments, the cell-based therapy is the adoptive transfer of autologous patient-derived cells. In various embodiments, the cell-based therapy is the adoptive transfer of allogenic donor-derived cells. In various embodiments, the cell-based therapy is the transfer of universal donor-derived or induced pluripotent stem cell-derived cells that are not patient specific and amenable to long-term storage. Such therapies are also referred to as ‘off-the-shelf’ therapies.

In various embodiments, the cancer therapeutic is a hormone therapy. In various embodiments, the cancer therapeutic comprises one or more antibody-drug conjugates. In various embodiments, the cancer therapeutic comprises one or more cancer vaccines. In various embodiments, the cancer vaccine is a protein, polypeptide, and/or nucleic acid vaccine.

In various embodiments, the cancer therapeutic is an immunotherapy selected from the group comprising oncolytic virus, bacteria, oncolytic bacteria or other bacterial consortia, tumor cell lysate, bacterial cell lysate, lipopolysaccharide (LPS), Bacillus Calmette-Guerin (BCG), a microbiome modulator, and/or a toll-like receptor (TLR) agonist. In various embodiments, the TLR agonist is a TLR3, TLR4, TLR5, TLR7, TLR8, TLR9, TLR10, TLR11, TLR12, and/or TLR13 agonist. In various embodiments, the TLR agonist is derived from virus, bacteria and/or made synthetically. In various embodiments, the immunotherapy is a STING pathway modulator.

In various embodiments, the cancer therapeutic comprises a viral or a bacterial vector. In various embodiments, the viral vector is selected from the group comprising adenovirus, adeno-associated virus (AAV), herpes simplex virus, lentivirus, retrovirus, alphavirus, flavivirus, rhabdovirus, measles virus, Newcastle disease virus, poxvirus, vaccinia virus, modified Ankara virus, vesicular stomatitis virus, picornavirus, tobacco mosaic virus, potato virus x, comovirus, or cucumber mosaic virus. In various embodiments, the virus is an oncolytic virus. In various embodiments the virus is a chimeric virus, a synthetic virus, a mosaic virus or a pseudotyped virus.

In various embodiments, the subject is a mammal. In various embodiments, the subject is a human.

In various embodiments, the negatively charged particles are formulated in a composition comprising a pharmaceutical acceptable excipient.

It is understood that each feature or embodiment, or combination, described herein is a non-limiting, illustrative example of any of the aspects of the invention and, as such, is meant to be combinable with any other feature or embodiment, or combination, described herein. For example, where features are described with language such as “various embodiments”, “one embodiment”, “some embodiments”, “certain embodiments”, “further embodiment”, “specific exemplary embodiments”, and/or “another embodiment”, each of these types of embodiments is a non-limiting example of a feature that is intended to be combined with any other feature, or combination of features, described herein without having to list every possible combination. Such features or combinations of features apply to any of the aspects of the disclosure. Where examples of values falling within ranges are disclosed, any of these examples are contemplated as possible endpoints of a range, any and all numeric values between such endpoints are contemplated, and any and all combinations of upper and lower endpoints are envisioned.

### BRIEF DESCRIPTION OF DRAWINGS

FIGS. 1A-C. Efficacy of negatively charged particles (referred to as ONP-302) when administered at different dose levels and at different dosing frequencies. MC38 tumor bearing mice were administered the indicated treatments and the indicated dosing frequencies beginning when tumor volumes were ˜50 mm³. (A and C) Effect of indicated treatments on tumor growth. (B) Effect of indicated treatments on survival. Significance of tumor growth kinetics were analyzed using two-way repeated measures ANOVA and Bonferroni's post-hoc analysis (*p=<0.05; **p<0.01; ****p<0.0001).

FIGS. 2A-B. Efficacy of ONP-302 When Administered at Different Dose Levels and at Different Dosing Frequencies in the B16-F10 tumor model. B16 tumor bearing mice were administered the indicated treatments when tumor volumes were ˜50 mm³. Effect of indicated treatments on (A) tumor growth and (B) survival are shown. Significance of tumor growth kinetics were analyzed using two-way repeated measures ANOVA and Bonferroni's post-hoc analysis (*p=<0.05; **p<0.01; ****p<0.0001).

FIG. 3. Efficacy of ONP-302 at inhibiting primary tumor growth in the presence and absence of a functional adaptive immune system in the orthotopic 4T1 tumor model. 4T1 primary tumors were implanted into the mammary fat pad of female WT or Rag1^{-/-} BALB/c mice. Day 1 post-tumor inoculation, animals were administered ONP-302 or Saline (Control) via intravenous tail vein injection once every 3 days. Shown are tumor volumes of mice in the indicated groups (N=10 per group). Significance of differences in tumor volumes was determined using multiple unpaired t-test (*p<0.05).

FIGS. 4A-D. Efficacy of ONP-302 at inhibiting primary tumor metastasis to the lungs in the presence and absence of a functional adaptive immune system in the orthotopic 4T1 tumor model. 4T1 primary tumors were implanted into the mammary fat pad of female WT or Rag1^{-/-} BALB/c mice. Day 1 post-tumor inoculation, animals were administered ONP-302 or Saline (Control) via intravenous tail vein injection once every 3 days. Mice were sacrificed on Day 21 and lung metastases were evaluated by assaying bioluminescence signal using IVIS®. Shown are incidence of lung metastasis in WT tumor bearing mice treated with (A) Saline or (B) ONP-302, and Rag1^{-/-} tumor bearing mice treated with (C) Saline (Control) or (D) ONP-302.

FIGS. 5A-B. Effect of ONP-302 on gene expression changes by scRNA-seq from lung tissue samples of orthotopic 4T1 tumor bearing mice. 4T1 tumor cells expressing luciferase were orthotopically implanted into the mammary fat pads of female 6-8-week-old female BALB/c mice. Mice were treated with ONP-302 or Saline once every three days beginning on Day 1 after tumor implantation. Mice were sacrificed on Day 21 post-tumor implantation and lungs were harvested and processed for scRNA-seq. (A) Numbers of different cell types in the lungs of ONP-302 and Saline treated mice. (B) Phenotypic changes in the indicate cell populations determined by Gene Set Enrichment Analysis (GSEA).

FIGS. 6A-C. Effect of ONP-302 on gene expression changes by scRNA-seq from spleens of naïve mice. Naïve female C57BL/6 mice (n=3 per group) were treated with saline or ONP-302 (1.0 mg/dose in 200 mL of saline) either once, three days consecutively (days 0, 1, and 2), or every three days (Days 0, 3, and 6) via i.v. injection. Twenty-four hours after the last injection, spleens were collected to assess the transcriptome via single cell RNAseq. (A) The distribution of cells present within the spleen in response to saline and each of the ONP-302 dosing schemes is compared. (B) The ONP-302-induced changes in gene expression by macrophages, monocytes, and neutrophils is compared. Red genes have a log fold change >1 and a p-value <0.05. (C) The signaling pathways induced by the ONP-302 treatment relative to saline treatment alone as measured through Gene Set Enrichment Analysis (GSEA), NES=normalized enrichment score, *p<0.1, **p<0.01, ***p<0.001, #pathway was not enriched in this sample.

FIGS. 7A-F. Effect of ONP-302 treatment on immune cells in the spleen of naïve mice. Naïve female C57BL/6 mice (n=3 per group) were treated with saline or ONP-302 (1.0 mg/dose in 200 mL of saline) either once, three days consecutively (days 0, 1, and 2), or every three days (Days 0, 3, and 6) via i.v. injection. Twenty-four hours after the last injection, spleens were collected to assess the immune cell populations present. A gating schematic detailing how cells were identified is shown (A). The total number of cells (B), as well as the phenotypes of CD4+ T cells, CD8+ T cells, NK cells (C), monocytes and macrophages (D), as well as other myeloid cells (E) are shown as heatmaps. The frequency of specific populations expressing cellular markers of activation after treatment is shown in (F). The data are presented as the mean percentage of cells+/−S.E.M. One representative experiment of two is presented. Asterisks (*, **, ***, ****) indicate a statistically significant difference as compared to saline treated mice, p<0.05, <0.01, <0.001, and <0.0001 respectively.

FIGS. 8A-B. Effect of ONP-302 treatment on cytokine/chemokine expression in RAW macrophages and bone marrow derived myeloid cells. RAW cells possessing or lacking the cGAS gene (cGAS −/−) were treated with ONP-302 for 24 hours in the presence or absence of a STING inhibitor (RU.521) or a Caspase inhibitor (ZVAD-FMK) and supernatants were collected to measure the level of chemokines and cytokines (A). BMDMs from either WT C57BL/6 mice or STING/mice were treated with ONP-302 for 24 hours and supernatants were collected to measure the level of chemokines and cytokines (B). Each dot represents a unique well of cells tested and the bar represents the mean+S.E.M. Asterisks (*, **, ***, ****) indicate a statistically significant difference as compared to untreated cells, p<0.05, <0.01, <0.001, and <0.0001 respectively.

### DETAILED DESCRIPTION

The present disclosure provides methods of dosing negatively charged particles free from attached drugs or bioactive agents, alone or in combination with one or more additional therapeutics, for the treatment of solid tumors in a subject in need thereof.

Each publication, patent application, patent, and other reference cited herein is incorporated by reference in its entirety to the extent that it is not inconsistent with the present disclosure.

It is noted here that as used in this specification and the appended claims, the singular forms “a,” “an,” and “the” include plural reference unless the context clearly dictates otherwise.

“Particle” as used herein refers to any non-tissue derived composition of matter, it may be a sphere or sphere-like entity, bead, or liposome. The term “particle”, the term “immune modifying particle”, and the term “bead” may be used interchangeably depending on the context. The term “negatively charged particles”, “surface functionalized particles’ and “negatively charged surface functionalized particles” may be used interchangeably depending on the context. Additionally, the term “particle” may be used to encompass beads and spheres.

“Negatively charged particle” as used herein refers to particles which possess a net surface charge that is less than zero. In embodiments, negatively charged particles are negatively charged surface functionalized particles. “Surface-functionalized” as used herein refers to particles which have one or more functional groups on its surface. In some embodiments, the surface functionalization occurs by the introduction of one or more functional groups to a surface of a particle. In embodiments, surface functionalization may be achieved by carboxylation (i.e., addition of one or more carboxyl groups to the particle surface) or addition of other chemical groups (e.g., other chemical groups that impart a negative surface charge).

“Free of therapeutic agent” or “free from attached drug” refers to negatively charged particles that do not comprise any other therapeutic agents or drugs. Said differently, therapeutically active drugs, peptides, antigenic moieties, or bioactive active agents are not attached, embedded, or otherwise associated with the negatively charged particles described herein. This phrase is intended to distinguish negatively charged particles coupled with therapeutic agents from the negatively charged particles described herein, which are themselves therapeutic agents.

Zeta potential is the charge that develops at the interface between a solid surface and its liquid medium. “Negative zeta potential” refers to a particle having a zeta potential of the particle surface as represented in milliVolts (mV) and measured by an instrument known in the field to calculate zeta potential, e.g., a NanoBrook ZetaPlus zeta potential analyzer or Malvern Zetasizer.

“Carboxylated particles” or “carboxylated beads” or “carboxylated spheres” includes any particle that has been modified or surface functionalized to add one or more additional carboxyl group onto the particle surface. Carboxylation of the particles can be achieved using any compound which adds additional carboxyl groups, including, but not limited to, Poly (ethylene-maleic anhydride) (PEMA). Carboxylation may also be achieved by using polymers with native carboxyl groups (e.g., PLGA) to form particles, in which the manufacturing process results in the additional carboxyl groups being located on the surface of the particle.

“Biodegradable” as used herein refers to a particle comprising a polymer that may undergo degradation, for example, by a result of functional groups reacting with the water in the solution. The term “degradation” as used herein refers to becoming soluble, either by reduction of molecular weight or by conversion of hydrophobic groups to hydrophilic groups. Biodegradable particles do not persist for long times in the body, and the time for complete degradation can be controlled. Biocompatible, biodegradable polymers useful in the present invention include polymers or copolymers of caprolactones, carbonates, amides, amino acids, orthoesters, acetals, cyanoacrylates, and degradable urethanes, as well as copolymers of these with straight chain or branched, substituted or unsubstituted, alkanyl, haloalkyl, thioalkyl, aminoalkyl, alkenyl, or aromatic hydroxy- or di-carboxylic acids. In addition, the biologically important amino acids with reactive side chain groups, such as lysine, arginine, aspartic acid, glutamic acid, serine, threonine, tyrosine and cysteine, or their enantiomers, may be included in copolymers with any of the aforementioned materials to provide reactive groups for conjugating to antigen peptides and proteins or conjugating moieties. Biodegradable materials suitable for the present invention include PLA, PGA, polypropylene sulfide, and PLGA polymers.

The term “subject” as used herein refers to a human or non-human animal, including a mammal or a primate, that is administered a particle as described herein. Subjects can include animals such as dogs, cats, rats, mice, rabbits, horses, pigs, sheep, cattle, and humans and other primates.

The term “therapeutic agent” refers to a moiety that is able to ameliorate or lessen one or more symptoms or signs of the disease or disorder being treated when administered at a therapeutically effective amount. Non-limiting examples of therapeutic agents include other cancer therapeutics, including peptides, proteins, or small molecule therapeutic agents.

The term “therapeutically effective amount” is used herein to indicate the amount of target-specific composition of the disclosure that is effective to ameliorate or lessen one or more symptoms or signs of the disease or disorder being treated.

The terms “treat”, “treated”, “treating” and “treatment”, as used with respect to methods herein refer to eliminating, reducing, suppressing, or ameliorating, either temporarily or permanently, either partially or completely, one or more clinical symptom, manifestation or progression of an event, disease, or condition. Such treating need not be absolute to be useful.

### Negatively Charged Particle

The present disclosure provides for use of negatively charged particles in the treatment methods described herein. In some embodiments, the negatively charged particles have a negative zeta potential. In some embodiments, the negative charge enhances phagocyte/monocyte uptake of the particles from circulation, for instance via receptor mediated phagocytosis including, but not limited to, interaction with scavenger receptors such as MARCO.

Negatively charged particles can be formed from a wide range of materials. The particle is preferably composed of a material suitable for biological use (e.g., a pharmaceutically acceptable material). For example, particles may be composed of glass, silica, polyesters of hydroxy carboxylic acids, polyanhydrides of dicarboxylic acids, or copolymers of hydroxy carboxylic acids and dicarboxylic acids. In various embodiments, the particles may be composed of polyesters of straight chain or branched, substituted or unsubstituted, saturated or unsaturated, linear or cross-linked, alkanyl, haloalkyl, thioalkyl, aminoalkyl, aryl, aralkyl, alkenyl, aralkenyl, heteroaryl, or alkoxy hydroxy acids, or polyanhydrides of straight chain or branched, substituted or unsubstituted, saturated or unsaturated, linear or cross-linked, alkanyl, haloalkyl, thioalkyl, aminoalkyl, aryl, aralkyl, alkenyl, aralkenyl, heteroaryl, or alkoxy dicarboxylic acids. Additionally, particles can be quantum dots, or composed of quantum dots, such as quantum dot polystyrene particles (Joumaa et al. (2006) Langmuir 22: 1810-6). Particles including mixtures of ester and anhydride bonds (e.g., copolymers of glycolic and sebacic acid) may also be employed. For example, particles may comprise materials including polyglycolic acid polymers (PGA), polylactic acid polymers (PLA), polysebacic acid polymers (PSA), poly(lactic-co-glycolic) acid copolymers (PLGA or PLG; the terms are interchangeable), poly(lactic-co-sebacic) acid copolymers (PLSA), poly(glycolic-co-sebacic) acid copolymers (PGSA), polypropylene sulfide polymers, poly(caprolactone), chitosan, etc. Other biocompatible, biodegradable polymers useful in the present invention include polymers or copolymers of caprolactones, carbonates, amides, amino acids, orthoesters, acetals, cyanoacrylates and degradable urethanes, as well as copolymers of these with straight chain or branched, substituted or unsubstituted, alkanyl, haloalkyl, thioalkyl, aminoalkyl, alkenyl, or aromatic hydroxy- or di-carboxylic acids. In addition, the biologically important amino acids with reactive side chain groups, such as lysine, arginine, aspartic acid, glutamic acid, serine, threonine, tyrosine and cysteine, or their enantiomers, may be included in copolymers with any of the aforementioned materials to provide reactive groups for conjugating to antigen peptides and proteins or conjugating moieties.

In embodiments, the negatively charged particles comprise one or more biodegradable polymers or materials. Biodegradable materials suitable for the present invention include PLA, PGA, polypropylene sulfide, and PLGA polymers.

In various embodiments, it is contemplated that the particles comprise polyglycolic acid polymers (PGA), polylactic acid (PLA), polystyrene, copolymers of PLG and PLA (poly(lactide-co-glycolide), PLGA), a liposome, PEG, cyclodextran, or a combination thereof.

In various embodiments, the negatively charged particle is a co-polymer having a molar ratio from about 100:0 to about 0:100, about 99:1 to about 1:99, about 80:20 to about 20:80, or about 50:50 polyglycolic acid:polylactic acid, including any values or ranges therebetween. In various embodiments, the negatively charged particle is a co-polymer having a molar ratio from about 50:50 or about 80:20 to about 99:1 polylactic acid:polyglycolic acid or from about 50:50 or about 80:20 to about 99:1 polyglycolic acid:polylactic acid. In some embodiments, the negatively charged surface functionalized particle is a poly(lactic-co-glycolic acid) particle. In various embodiments, the negatively charged particle comprises 50:50 polylactic acid:polyglycolic acid. In various embodiments, the negatively particle comprises polylactic acid:polyglycolic acid from about 99:1 to about 1:99, e.g., about 99:1, about 95:5, about 90:10, about 85:15, about 80:20, about 75:25, about 70:30, about 65:35, about 60:40, about 55:45, about 50:50, about 45:55, about 40:60, about 35:65, about 30:70, about 25:75, about 20:80, about 15:85, about 10:90, about 5:95, and about 1:99, including all values and ranges that lie in between these values.

In some embodiments, the zeta potential of the negatively charged particle is from about −100 mV to about −1 mV. In some embodiments, the zeta potential of the negatively charged particle is from about −100 mV to about −40 mV, from about −80 mV to about −30 mV, from about −75 mV to about −40 mV, from about −70 mV to about −30 mV, from about −60 mV to about −35 mV, or from about −50 mV to about −40 mV, including any values or ranges therebetween. In various embodiments, the zeta potential is about −30 mV, about −35 mV, about −40 mV, about −45 mV, about −50 mV, about −55 mV, about −60 mV, about −65 mV, about −70 mV, about −75 mV, about −80 mV, about −85 mV, about −90 mV, about −95 mV or about −100 mV, including all values and subranges that lie between these values.

In some embodiments, the negatively charged particles have an average diameter of about 0.1 μm to about 10 μm. In some embodiments, the negatively charged particles have an average diameter of about 0.2 μm to about 2 μm. In some embodiments, the negatively charged particles have an average diameter of about 0.3 μm to about 5 μm. In some embodiments, the negatively charged particles have an average diameter of about 0.5 μm to about 3 μm. In some embodiments, the negatively charged particles have an average diameter of about 0.5 μm to about 1 μm. In some embodiments, the negatively charged particles have an average diameter of about 100 nm to 1500 nm, about 200 nm and 2000 nm, about 100 nm to 10000 nm, about 300 nm to 1000 nm, about 400 nm to 800 nm, or about 200 nm to 700 nm, including all values and subranges that lie between these values.

In various embodiments, the negatively charged particles have a homogenous size distribution. In various embodiments, the negatively charged particles have a homogenous size distribution wherein at least 90% of the negatively charged particles have an average diameter ranging from about 0.05 μm to about 10 μm, about 0.1 μm to about 10 μm, about 0.1 μm to about 5 μm, about 0.1 μm to about 3 μm, about 0.3 μm to about 5 μm, or about 0.3 μm to about 3 μm, including any values or ranges therebetween. In various embodiments, the negatively charged particles have a homogenous size distribution wherein at least 90% of the negatively charged particles have an average diameter of about 100 nm to about 10000 nm, about 100 nm to about 5000 nm, about 100 nm to about 3000 nm, about 100 nm to about 2000 nm, about 300 nm to about 5000 nm, about 300 nm to about 3000 nm, about 300 nm to about 1000 nm, about 300 nm to about 800 nm, about 400 nm to about 800 nm, or about 200 nm to about 700 nm, including any values or ranges therebetween. In various embodiments, the negatively charged particles have an average diameter of about 50 nm, about 100 nm, about 200 nm, about 300 nm, about 400 nm, about 500 nm, about 600 nm, about 700 nm, about 800 nm, about 900 nm, about 1000 nm, about 1100 nm, about 1200 nm, about 1300 nm, about 1400 nm, about 1500 nm, or about 2000 nm, or any values or ranges therebetween. In various embodiments, the negatively charged particles have a homogenous size distribution wherein at least 50% of the negatively charged particles have an average diameter ranging from about 0.05 μm to about 10 μm, about 0.1 μm to about 10 μm, about 0.1 μm to about 5 μm, about 0.1 μm to about 3 μm, about 0.3 μm to about 5 μm, and about 0.3 μm to about 3 μm, including any values or ranges therebetween. In various embodiments, the negatively charged particles have a homogenous size distribution wherein at least 50% of the negatively charged particles have a diameter of about 100 nm to about 10000 nm, about 100 nm to about 5000 nm, about 100 nm to about 3000 nm, about 100 nm to about 2000 nm, about 300 nm to about 5000 nm, about 300 nm to about 3000 nm, about 300 nm to about 1000 nm, about 300 nm to about 800 nm, about 400 nm to about 800 nm, or about 200 nm to about 700 nm, including all values and ranges therebetween. In various embodiments, the negatively charged particles have an average diameter of about 50 nm, about 100 nm, about 200 nm, about 300 nm, about 400 nm, about 500 nm, about 600 nm, about 700 nm, about 800 nm, about 900 nm, about 1000 nm, about 1100 nm, about 1200 nm, about 1300 nm, about 1400 nm, about 1500 nm, or about 2000 nm, including any values or ranges therebetween. In various embodiments, the negatively charged particles have a homogenous size distribution wherein at least 10% of the negatively charged particles have an average diameter ranging from about 0.05 μm to about 10 μm, about 0.1 μm to about 10 μm, about 0.1 μm to about 5 μm, about 0.1 μm to about 3 μm, about 0.3 μm to about 5 μm, and about 0.3 μm to about 3 μm, including any values or ranges therebetween. In various embodiments, the negatively charged particles have a homogenous size distribution wherein at least 10% of the negatively charged particles have an average diameter of about 100 nm to about 10000 nm, about 100 nm to about 5000 nm, about 100 nm to about 3000 nm, about 100 nm to about 2000 nm, about 300 nm to about 5000 nm, about 300 nm to about 3000 nm, about 300 nm to about 1000 nm, about 300 nm to about 800 nm, about 400 nm to about 800 nm, or about 200 to about 700 nm, including any values or ranges therebetween. In various embodiments, the negatively charged particles have an average diameter of about 50 nm, about 100 nm, about 200 nm, about 300 nm, about 400 nm, about 500 nm, about 600 nm, about 700 nm, about 800 nm, about 900 nm, about 1000 nm, about 1100 nm, about 1200 nm, about 1300 nm, about 1400 nm, about 1500 nm, or about 2000 nm, including any values or ranges there between.

In embodiments, the negatively charged particles have a D90 of about 300 nm to about 1000 nm, including about 1000 nm, about 900 nm, about 800 nm, about 700 nm, about 600 nm, about 300 nm, about 310 nm, about 320 nm, about 330 nm, about 340 nm, about 350 nm, about 360 nm, about 370 nm, about 380 nm, about 390 nm, about 400 nm, about 410 nm, about 420 nm, about 430 nm, about 440 nm, about 450 nm, about 460 nm, about 470 nm, about 480 nm, about 490 nm about about 500 nm, about 510 nm, about 520 nm, about 530 nm, about 540 nm, about 550 nm, about 560 nm, about 570 nm, about 580 nm, about 590 nm, about 600 nm, about 610 nm, about 620 nm, about 630 nm, about 640 nm, about 650 nm, about 660 nm, about 670 nm, about 680 nm, about 690 nm, or about 700 nm, including any values or ranges therebetween. In embodiments, the negatively charged particles have a D50 of about 400 nm to about 600 nm, including about about 400 nm, about 420 nm, about 440 nm, about 460 nm, about 480 nm, about 500 nm, about 510 nm, about 520 nm, about 530 nm, about 540 nm, about 550 nm, about 560 nm, about 570 nm, about 580 nm, about 590 nm, or about 600 nm, including any values or ranges therebetween. In embodiments, the negatively charged particles have a D10 of about 300 nm to about 550 nm, including about 300 nm, about 320 nm, about 340 nm, about 360 nm, about 380 nm, about 400 nm, about 410 nm, about 420 nm, about 430 nm, about 440 nm, about 450 nm, about 460 nm, about 470 nm, about 480 nm, about 490 nm, about 500 nm, about 510 nm, about 520 nm, about 530 nm, about 540 nm, or about 550 nm, including any values or ranges therebetween. In embodiments, the negatively charged particles have a D90 of about 600 nm to about 700 nm. In embodiments, the negatively charged particles have a D50 of about 550 nm to about 600 nm. In embodiments, the negatively charged particles have a D10 of about 500 nm to about 550 nm.

To administer negatively charged particles as described herein to human or other mammals, the particle may be formulated in a sterile composition comprising one or more sterile pharmaceutically acceptable carriers. The phrase “pharmaceutically or pharmacologically acceptable” refer to molecular entities and compositions that do not produce allergic, or other adverse reactions when administered using routes well-known in the art, as described below. “Pharmaceutically acceptable carriers” include any and all clinically useful solvents, dispersion media, coatings, antibacterial and antifungal agents, isotonic and absorption delaying agents and the like.

Pharmaceutical compositions of the present disclosure containing a negatively charged surface functionalized particle herein may contain sterile pharmaceutically acceptable carriers or additives depending on the route of administration. Examples of such carriers or additives include water, a pharmaceutical acceptable organic solvent, collagen, polyvinyl alcohol, polyvinylpyrrolidone, a carboxyvinyl polymer, carboxymethylcellulose sodium, polyacrylic sodium, sodium alginate, water-soluble dextran, carboxymethyl starch sodium, pectin, methyl cellulose, ethyl cellulose, xanthan gum, gum Arabic, casein, gelatin, agar, diglycerin, glycerin, propylene glycol, polyethylene glycol, Vaseline, paraffin, stearyl alcohol, stearic acid, human serum albumin (HSA), mannitol, sorbitol, lactose, a pharmaceutically acceptable surfactant and the like. Additives used are chosen from, but not limited to, the above or combinations thereof, as appropriate, depending on the dosage form of the present invention. For solutions or emulsions, suitable carriers include, for example, aqueous or alcoholic/aqueous solutions, emulsions or suspensions, including saline and buffered media. Parenteral vehicles can include sodium chloride solution, Ringer's dextrose, dextrose and sodium chloride, lactated Ringer's or fixed oils. Intravenous vehicles can include various additives, preservatives, or fluid, nutrient or electrolyte replenishers. A variety of aqueous carriers are suitable, e.g., sterile phosphate buffered saline solutions, bacteriostatic water, water, buffered water, 0.4% saline, 0.3% glycine, and the like, and may include other proteins for enhanced stability, such as albumin, lipoprotein, globulin, etc., subjected to mild chemical modifications or the like.

A variety of aqueous carriers, e.g., sterile phosphate buffered saline solutions, bacteriostatic water, water, buffered water, 0.4% saline, 0.3% glycine, and the like, and may include other proteins for enhanced stability, such as albumin, lipoprotein, globulin, etc., subjected to mild chemical modifications or the like.

Therapeutic formulations of the inhibitors are prepared for storage by mixing the inhibitor having the desired degree of purity with optional physiologically acceptable carriers, excipients or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl para-bens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

The formulations to be used for in vivo administration must be sterile. This is readily accomplished by filtration through sterile filtration membranes.

Aqueous suspensions may contain the active compound in admixture with excipients suitable for the manufacture of aqueous suspensions. Such excipients are suspending agents, for example sodium carboxymethylcellulose, methylcellulose, hydroxypropylmethylcellulose, sodium alginate, polyvinylpyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethyl-eneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate.

Solid dosage forms for oral administration include capsules, tablets, pills, powders, and granules. In such solid dosage forms, the modified particles are mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

It is contemplated that the particle may further comprise a surfactant and/or stabilizer. The surfactant and/or stabilizer can be anionic, cationic, or nonionic. Surfactants in the poloxamer and polaxamine family are commonly used in particle synthesis. Surfactants and/or stabilizers that may be used, include, but are not limited to polyethylene glycol (PEG), Tween-80, gelatin, dextran, pluronic L-63, polyvinyl alcohol (PVA), polyacrylic acid (PAA), methylcellulose, lecithin, Dimethylaminobenzaldehyde (DMAB) and poly(ethylene-alt-maleic anhydride) (PEMA). Additionally, biodegradable and biocompatible surfactants including, but not limited to, vitamin E TPGS (D-α-tocopheryl polyethylene glycol 1000 succinate), poly amino acids (e.g., polymers of lysine, arginine, aspartic acid, glutamic acid, serine, threonine, tyrosine and cysteine, or their enantiomers), and sulfate polymers. In certain embodiments, two surfactants are used. In certain embodiments, two stabilizers are used. In certain embodiments, a combination of two or more surfactants and stabilizers are used. For example, if the particle is produced by a double emulsion method, the two surfactants can include a hydrophobic surfactant for the first emulsion, and a hydrophobic surfactant for the second emulsion. For example, stabilizers can be compounds which stabilize the primary and/or the secondary emulsion as described herein by providing a physical barrier or an energy barrier between adjacent nanoparticle droplets in the emulsion, thereby reducing their probability to coalesce and form larger nanoparticle droplets.

Emulsions occur in many forms of processing. Oil-water (single) or water-oil-water (double) emulsion are methods by which negatively charged surface functionalized PLGA particles can be manufactured. In summary, PLGA is dissolved into an organic phase (oil) that is emulsified with a surfactant or stabilizer (water). High intensity homogenization (e.g., sonication bursts) facilitate the formation of small polymer droplets. The resulting emulsion is added to a larger aqueous phase and stirred for several hours, which allows the solvent to evaporate. Hardened nanoparticles are collected and washed by centrifugation. In certain embodiments, hardened emulsion particles can be obtained through evaporation of the oil phase.

“Water-in-oil-in-water” (W/O/W) emulsion is an example of a double emulsion, in which dispersions of small water droplets within larger oil droplets are themselves dispersed in a continuous aqueous phase. Because of their compartmentalized internal structure, double emulsions can provide advantages over simple oil-in-water emulsions for encapsulation, such as the ability to carry both polar and non-polar cargos (pharmaceutical/biological agent, e.g., proteins), and improved control over release of therapeutic molecules. The preparation of double emulsions typically requires surfactants or their mixtures for stability. The surfactants stabilize droplets subjected to extreme flow, leading to direct, mass production of robust double nanoemulsions that are amenable to nanostructured encapsulation applications in various industries. In one example, a double emulsion process involves generating a primary emulsion by mixing an aqueous solution including a polymer resulting in a water-in-oil primary emulsion. The primary emulsion is then mixed with a solution including one or more surfactants to form an oil-in-water secondary emulsion. The secondary emulsion is then hardened by evaporation to remove the solvent(s) resulting in hardened polymeric nanoparticles encapsulating the pharmaceutical/biological agent(s).

“Homogenization” as used herein relates to an operation using a class of processing equipment referred to as homogenizers that are geared towards reducing the size of droplets in liquid-liquid dispersions. Factors that affect the particle or droplet size include but are not limited to the type of emulsifier, emulsifier concentration, solution conditions, and mechanical device (homogenizing power; pressure, rotation speed, time). Non-limiting examples of homogenizers include high speed blender, high pressure homogenizers, colloid mill, high shear dispersers, ultrasonic disruptor membrane homogenizers, and ultrasonicators. Mechanical homogenizers, manual homogenizers, sonicators, mixer mills, vortexers, and the like may be utilized for mechanical and physical disruption within the scope of the disclosure.

### Methods of Use

Provided herein is a method of treating solid tumors in a subject comprising administering to the subject negatively charged particles, wherein the negatively charged particles are administered at a dose of about 0.1 mg/kg to about 15 mg/kg. Also provided herein is a method of inducing an anti-tumor immune response in a subject suffering from solid tumors, the method comprising administering to the subject negatively charged particles, wherein the negatively charged particles are administered at a dose of about 0.1 mg/kg to about 15 mg/kg. In various embodiments, the negatively charged particles are administered at a dose of about 0.5 mg/kg to about 10 mg/kg, from about 1 mg/kg to 8 mg/kg, from about 1.5 mg/kg to 15 mg/kg, from about 2 mg/kg to 15 mg/kg, from about 2 mg/kg to 10 mg/kg, from about 3 mg/kg to 10 mg/kg, from about 4 mg/kg to 10 mg/kg, from about 4 mg/kg to 15 mg/kg, from about 5 mg/kg to 15 mg/kg, or from about 2 mg/kg to 8 mg/kg. In various embodiments, negatively charged particles are administered at a dose of about 0.1 mg/kg, 0.25 mg/kg, 0.5 mg/kg, 1.0 mg/kg, 2.0 mg/kg, 3.0 mg/kg, 4.0 mg/kg, 5 mg/kg, 6 mg/kg, 8.0 mg/kg, 10 mg/kg, 12 mg/kg, or 15 mg/kg. In various embodiments, negatively charged particles are administered at a dose of about 10 mg, 25 mg, 50 mg, 75 mg, 100 mg, 125 mg, 150 mg, 175 mg, 200 mg, 225 mg, 250 mg, 275 mg, 300 mg, 325 mg, 350 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg, 800 mg, 850 mg, 875 mg, 900 mg, 950 mg, 975 mg, or 1000 mg.

In various embodiments, negatively charged particles are administered intravenously, subcutaneously, intramuscularly, intraperitoneally, intranasally, or orally.

In various embodiments, negatively charged particles are administered at a concentration of about 0.05 mg/mL to about 50 mg/mL. In various embodiments, negatively charged particles are administered at a concentration of about 0.1 mg/mL, 0.5 mg/mL, 1 mg/mL, 2 mg/mL, 3 mg/mL, 4 mg/mL, 5 mg/mL, 6 mg/mL, 7 mg/mL, 8 mg/mL, 9 mg/mL, 10 mg/mL, 11 mg/mL, 12.5 mg/mL, 15 mg/mL, 17.5 mg/mL, 20 mg/mL, 25 mg/mL, 30 mg/mL, 40 mg/mL, or 50 mg/mL. In various embodiments, negatively charged particles are administered via intravenous infusion lasting about 1, 2, 3, 4, 5, 6, 7, or 8 hours.

In various embodiments, the negatively charged particles are administered once daily, twice daily, three times per day, seven times per week, six times per week, five times per week, four times per week, three times per week, twice weekly, once weekly, once every two weeks, once every three weeks, once every 4 weeks, once every two months, once every three months, once every 6 months or once per year. In various embodiments, the negatively charged surface functionalized particle and/or the cancer therapeutic is administered for 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, or 52 weeks, or more.

In various embodiments, the negatively charged particles are administered in treatment cycles. In various embodiments, one treatment cycle consists of 1, 2, 3, 4, 5, 6, or 7 doses administered weekly. In various embodiments, the subject is administered 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, or 52 treatment cycles of negatively charged particles.

Exemplary diseases, conditions or disorders that can be treated using the methods herein include cancers, such as esophageal cancer, pancreatic cancer, metastatic pancreatic cancer, metastatic adenocarcinoma of the pancreas, bladder cancer, stomach cancer, fibrotic cancer, glioma, malignant glioma, diffuse intrinsic pontine glioma, recurrent childhood brain neoplasm renal cell carcinoma, clear-cell metastatic renal cell carcinoma, kidney cancer, prostate cancer, metastatic castration resistant prostate cancer, stage IV prostate cancer, metastatic melanoma, melanoma, malignant melanoma, recurrent melanoma of the skin, melanoma brain metastases, stage IIIA skin melanoma; stage IIIB skin melanoma, stage IIIC skin melanoma; stage IV skin melanoma, malignant melanoma of head and neck, lung cancer, non small cell lung cancer (NSCLC), squamous cell non-small cell lung cancer, breast cancer, recurrent metastatic breast cancer, hepatocellular carcinoma, hodgkin's lymphoma, follicular lymphoma, non-hodgkin's lymphoma, advanced B-cell NHL, HL including diffuse large B-cell lymphoma (DLBCL), multiple myeloma, chronic myeloid leukemia, adult acute myeloid leukemia in remission; adult acute myeloid leukemia with Inv(16)(p13.1q22); CBFB-MYH11; adult acute myeloid leukemia with t(16;16)(p13.1;q22); CBFB-MYH11; adult acute myeloid leukemia with t(8;21)(q22;q22); RUNX1-RUNX1T1; adult acute myeloid leukemia with t(9;11)(p22;q23); MLLT3-HLL; adult acute promyelocytic leukemia with t(15;17)(q22;q12); PML-RARA; alkylating agent-related acute myeloid leukemia, chronic lymphocytic leukemia, richter's syndrome; waldenstrom macroglobulinemia, adult glioblastoma; adult gliosarcoma, recurrent glioblastoma, recurrent childhood rhabdomyosarcoma, recurrent ewing sarcoma/peripheral primitive neuroectodermal tumor, recurrent neuroblastoma; recurrent osteosarcoma, colorectal cancer, MSI positive colorectal cancer; MSI negative colorectal cancer, nasopharyngeal nonkeratinizing carcinoma; recurrent nasopharyngeal undifferentiated carcinoma, cervical adenocarcinoma; cervical adenosquamous carcinoma; cervical squamous cell carcinoma; recurrent cervical carcinoma; stage IVA cervical cancer; stage IVB cervical cancer, anal canal squamous cell carcinoma; metastatic anal canal carcinoma; recurrent anal canal carcinoma, recurrent head and neck cancer; carcinoma, squamous cell of head and neck, head and neck squamous cell carcinoma (HNSCC), ovarian carcinoma, colon cancer, gastric cancer, advanced GI cancer, gastric adenocarcinoma; gastroesophageal junction adenocarcinoma, bone neoplasms, soft tissue sarcoma; bone sarcoma, thymic carcinoma, urothelial carcinoma, recurrent merkel cell carcinoma; stage III merkel cell carcinoma; stage IV merkel cell carcinoma, myelodysplastic syndrome and recurrent mycosis fungoides and Sezary syndrome. In various embodiments, the cancers are selected from brain cancer, skin cancer, eye cancer, breast cancer, prostate cancer, lung cancer, esophageal cancer, head and neck cancer, cervical cancer, liver cancer, colon cancer, bone cancer, uterine cancer, ovarian cancer, bladder cancer, stomach cancer, oral cancer, thyroid cancer, kidney cancer, testicular cancer, leukemia, lymphoma, and mesothelioma.

In various embodiments, the disclosure provides a method of treating cancer in a subject comprising administering to the subject negatively charged particles alone or in combination with a cancer therapeutic, wherein the subject has experienced disease progression after all prior lines of standard of care therapy or cannot tolerate standard of care therapy. Standard of care therapies may involve multiple lines of treatment including surgery, cytotoxic agents, immunotherapies, and cell therapies. In various embodiments, the subject has received and experienced disease progression on 1, 2, 3, 4, 5, or more prior lines of therapy.

In various embodiments, the disclosure provides a method of treating cancer in a subject comprising administering to the subject negatively charged particles alone or in combination with a cancer therapeutic, wherein the subject has experienced tumor recurrence after surgical resection of the primary tumor. In various embodiments, the disclosure provides a method of treating cancer in a subject comprising administering to the subject negatively charged particles alone or in combination with a cancer therapeutic, wherein the subject has a tumor that cannot be surgically removed. In various embodiments, the disclosure provides a method of treating cancer in a subject comprising administering to the subject negatively charged particles alone or in combination with a cancer therapeutic, wherein the subject has no treatment options available.

Multiple tools for the diagnosis of cancer in a subject have been described and are incorporated herein by reference. Examples of tools for detecting and diagnosing the presence of cancer in a subject include Computed Tomography (CT), Magnetic Resonance Imaging (MRI), Positron Emission Tomography (PET), Single-photon Emission Computerized Tomography (SPECT), ultrasound, X-ray imaging, digital mammography, and sentinel node mapping. In various embodiments, the method of detecting and diagnosing cancer in a subject includes obtaining and characterizing one or more biological samples from the subject. In various embodiments, the biological sample is blood, tumor biopsy, tissue biopsy, cerebrospinal fluid, urine, stool, buccal swab, nasal swab, lavage, and bone marrow biopsy.

Examples of approved diagnostics include FOUNDATIONONE® CDX, FOUNDATIONONE® LIQUID, FOUNDATIONONE® HEME, BRACAnalysis CDx, therascreen EGFR RGQ PCR kit, cobase EGFR Mutation Test v2, PD-L1 IHC 22C3 pharmDx, Abbott Real Time IDH1, MRDx BCR-ABL test, VENTANA ALK (D5F3) CDx Assay, Abbott RealTime IDH2, Praxis Extended RAS Panel, Oncomine Dx Target Test, LeukoStrat CDx FLT3 Mutation Assay, FoundationFocus CDxBRCA Assay, Vysis CLL FISH Probe Kit, KIT D816V Mutation Detection, PDGFRB FISH, cobas KRAS Mutation Test, therascreen KRAS RGQ PCR Kit, FerriScan, Dako c-KIT pharmDx, INFORM Her-2/neu, PathVysion HER-2 DNA Probe Kit, SPOT-LIGHT HER2 CISH Kit, Bond Oracle HER2 IHC System, HER2 CISH pharmDx Kit, INFORM HER2 DUAL ISH DNA Probe Cocktail, HercepTest, HER2 FISH pharmDx Kit, THXID BRAF Kit, Vysis ALK Break Apart FISH Probe Kit, cobas 4800 BRAF V600 Mutation Test, VENTANA PD-L1 (SP142) Assay, therascreen FGFR RGQ RT-PCR Kit, and therascreen PIK3CA RGQ PCR Kit.

In various embodiments, the disclosure of the present invention provides a method of treating cancer in a subject, the method comprising administration to the subject negatively charged particles, alone or in combination with one or more cancer therapeutics.

In various embodiments, the administration of negatively charged particles reduces the tumor size or tumor burden by about 1%, 2%, 3%, 4%, 5%, 6%, 7%, 8%, 9%, 10%, 20%, 30% or more. In various embodiments, the administration reduces the tumor size or tumor burden by about 10%, about 20%, about 30%, about 40%, about 50%, about 60%, about 70%, about 80%, about 90% or about 100%, including all values and ranges in between these values. Several criteria and definitions published in the literature can be used to determine the effect of one or more treatments on tumors in a subject suffering from cancer. Based on these criteria, tumors are defined as ‘responsive’, ‘stable’, or ‘progressive’ when they improve, remain the same, or worsen during the course of treatment, respectively. Examples of the most commonly used criteria published in the literature include Response Evaluation Criteria in Solid Tumors (RECIST), Modified Response Evaluation Criteria in Solid Tumors (mRECIST), PET Response Criteria in Solid Tumors (PERCIST), Choi Criteria, Lugano Response Criteria, European Association for the Study of the Liver (EASL) Criteria, Response Evaluation Criteria in the Cancer of the Liver (RECICL), and WHO Criteria in Tumor Response.

In various embodiments, the administration of negatively charged particles in a subject suffering from cancer demonstrates therapeutic efficacy as defined by the RECIST criteria. In various embodiments, therapeutic efficacy is calculated as objective response rate (ORR) equal to the sum of complete responses (CR) and partial responses (PR) observed for each dose level divided by the total number of subjects (N) dosed at that level. In various embodiments, the tumor response criteria according to RECIST as defined as follows based on evaluation of target lesions:
Complete Response (CR): Disappearance of all target lesions. Any pathological lymph nodes (whether target or non-target) must have reduction in short axis to <1 cm.
Partial Response (PR): At least a 30% decrease in the sum of diameters of target lesions, taking as reference the Baseline sum diameters.
Progressive Disease (PD): At least a 20% increase in the sum of diameters of target lesions, taking as reference the smallest sum on study (this includes the Baseline sum if that is the smallest on study). In addition to the relative increase of 20%, the sum must also demonstrate an absolute increase of at least 0.5 cm. The appearance of 1 or more new lesions is also considered progression.
Stable Disease (SD): Neither sufficient shrinkage to qualify for PR nor sufficient increase to qualify for PD, taking as reference the smallest sum diameters while on study.

In various embodiments, the tumor response criteria according to RECIST are defined as follows based on evaluation of non-target lesions:
Complete Response (CR): Disappearance of all non-target lesions and normalization of tumor marker level. All lymph nodes must be non-pathological in size (<1 cm short axis).
Non-complete Response/Non-progressive Disease: Persistence of 1 or more non-target lesion(s) and/or maintenance of tumor marker level above the normal limits.
Progressive Disease (PD): Unequivocal progression of existing non-target lesions.

In various embodiments, a measurable lesion is defined as one that can be accurately measured in at least 1 dimension (longest diameter in the plane of measurement is to be recorded) with a minimum size of 1 cm by CT scan (CT scan slice thickness no greater than 0.5 cm), or 1 cm caliper measurement by clinical examination (lesions which cannot be accurately measured with calipers should be recorded as non-measurable), or 2 cm by chest x-ray.

In various embodiments, a non-measurable lesion is defined as all other lesions, included small lesions (longest diameter <1.0 cm or pathological lymph nodes with ≥10 to <1.5 cm short axis) as well as truly non measurable lesions. Lesions considered truly non-measurable include: leptomeningeal disease, ascites, pleural or pericardial effusion, inflammatory breast disease, lymphangitic involvement of skin or lung, abdominal masses/abdominal organomegaly identified by physical examination that is not measurable by reproducible imaging techniques.

In various embodiments, administration of negatively charged particles improves survival. In various embodiments, administration of negatively charged particles improves progression free survival and/or overall survival. In various embodiments, administration of negatively charged particles improves survival by 0.5 to 12 months. In various embodiments, administration of negatively charged particles improves survival by 0.5, 0.6, 0.7, 0.8, 0.9, 1, 2, 3, 4, 5, 6, 7, 8, 9, 10, 11 or 12 months including all values and ranges in between these values. In various embodiments, administration of negatively charged particles improves survival by 1 to 100 years. In various embodiments, administration of negatively charged particles improves survival by 1 to 50 years. In various embodiments, administration of negatively charged particles improves survival by 1, 2, 3, 4, 5, 6, 7, 8, 9, 10, 15, 20, 25, 30, 35, 40, 45, 50, 55, 60, 65, 70, 75, 80, 85, 90, 95, or 100 years including all values and ranges in between these values.

In various embodiments, administration of negatively charged particles reduces the number of circulating tumor cells (CTCs) in blood. In various embodiments, administering the negatively charged particles reduces the number of CTCs in by about 1%, 2%, 3%, 4%, 5%, 6%, 7%, 8%, 9%,10%, 20%, 30% or more. In various embodiments, the administration reduces the number of CTCs by about 10%, about 20%, about 30%, about 40%, about 50%, about 60%, about 70%, about 80%, about 90% or about 100%, including all values and ranges in between these values. In various embodiments, administration of negatively charged particles reduces the number of CTC clusters. In various embodiments, administration of negatively charged particles reduces the number of CTC clusters in blood by about 1%, 2%, 3%, 4%, 5%, 6%, 7%, 8%, 9%,10%, 20%, 30% or more. In various embodiments, the administration reduces the number of CTC clusters by about 10%, about 20%, about 30%, about 40%, about 50%, about 60%, about 70%, about 80%, about 90% or about 100%, including all values and ranges in between these values. In various embodiments, administration of negatively charged particles reduces the number of CTC-leukocyte clusters. In various embodiments, administration of negatively charged particles reduces the number of CTC-leukocyte clusters in blood by about 1%, 2%, 3%, 4%, 5%, 6%, 7%, 8%, 9%,10%, 20%, 30% or more. In various embodiments, the administration reduces the number of CTC-leukocyte clusters by about 10%, about 20%, about 30%, about 40%, about 50%, about 60%, about 70%, about 80%, about 90% or about 100%, including all values and ranges in between these values. In various embodiments, the levels of CTCs and/or CTC-leukocyte clusters in blood are reduced to ≤20, ≤15, ≤10, ≤5, ≤1, or 0 per 7.5 mL blood inclusive of inclusive of all values and ranges between these values. In various embodiments, the leukocyte in the CTC-leukocyte cluster is a myeloid derived cell. In various embodiments, the myeloid derived cell is a monocyte, macrophage, neutrophil, or a dendritic cell. In various embodiments, the number of CTCs and CTC-leukocyte clusters in blood are enumerated per mL of blood. In various embodiments, the number of CTCs and CTC-leukocyte clusters in blood are determined using commercially available size-based and/or tumor marker based microfluidic capture devices.

In various embodiments, administration of negatively charged particles to a subject in need thereof induces an anti-tumor immune response. In various embodiments, the anti-tumor immune response is a T cell response, a B cell response, an NK cell response, an NKT cell response, and/or a myeloid cell response. In various embodiments, the myeloid cells are selected from the group consisting of monocytes, macrophages, dendritic cells, and neutrophils. In various embodiments, the anti-tumor response is a cytokine and/or a chemokine response. In various embodiments, the induction of the anti-tumor response is assayed from one or more biological samples obtained from the subject prior to treatment (baseline), during treatment, and/or after the completion of treatment. In various embodiments, the biological sample is selected from the group consisting whole-blood, peripheral blood, peripheral blood mononuclear cells (PBMCs), serum, plasma, urine, cerebrospinal fluid (CSF), stool, a tissue biopsy, and/or a bone-marrow biopsy.

In various embodiments, administration of negatively charged particles to a subject in need thereof increases the levels of activated anti-tumor immune cells. In various embodiments, the levels of activated anti-tumor immune cells are increased by 5-100% (e.g., increased by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, or 50% compared to one or more blood and/or tumor samples collected from the subject prior to treatment. In various embodiments, the activated pro-inflammatory cells are dendritic cells (DCs), macrophages, M1 macrophages, T-cells, B-cells, NK cells, NK-T cells, and iNK cells. In various embodiments, the frequency of pro-inflammatory immune cells is increased to 10-50% (e.g., about 10%, 15%, 20%, 25%, 30%, 35%, 40%, 45%, or 50%, inclusive of all values and ranges between these values) of all leukocytes analyzed from one or more blood samples collected from the subject. In various embodiments, activated pro-inflammatory immune cells are identified by the assay of cell-surface protein expression.

In various embodiments, administration of negatively charged particles to a subject in need thereof decreases the levels of pro-tumor cells. In various embodiments, the levels of pro-tumor immune cells are reduced by 5-100% (e.g reduced by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, or 50% compared to one or more blood and/or tumor samples collected from the subject prior to treatment. In various embodiments, the levels of immune suppressive cells are reduced by about 2-100 fold (e.g., about 2, 5, 10, 15, 20, 25, 30, 35, 40, 45, 50, 55, 60, 65, 70, 75, 80, 85, 90, 95, or 100 fold, inclusive of all values and ranges between these values) compared to one or more blood samples collected from the subject prior to treatment. In various embodiments, immune suppressive cells are identified by the assay of cell-surface proteins expression. In various embodiments, the pro-tumor cells are myeloid derived suppressor cells (MDSCs), tumor-associated macrophages (TAMs), neutrophils, T_{reg} cells, and B_{reg} cells. In various embodiments, MDSCs are monocytic MDSCs (M-MDSCs) and polymorphonuclear MDSCs (PMN-MDSCs). In various embodiments, the TAMs are M2 TAMs. In various embodiments, the immune suppressive cells are CAFs.

In various embodiments, the cell surface proteins are selected from the group consisting receptor tyrosine kinase (RTK), CD1C, CD2, CD3, CD4, CD5, CD8, CD9, CD10, CD11B, CD11C, CD14, CD15, CD16, CD18, CD19, CD20, CD21, CD22, CD23, CD24, TACI, CD25, CD27, CD28, CD30, CD30L, CD31, CD32, CD32B, CD34, CD33, CD38, CD39, CD40, CD40-L, CD41B, CD42A, CD42B, CD43, CD44, CD45, CD47, CD45RA, CD45RO, CD48, CD52, CD55, CD56, CD58, CD61, CD66B, CD70, CD72, CD79, CD68, CD84, CD86, CD93, CD94, CD95, CRACC, BLAME, BCMA, CD103, CD107, CD112, CD120A, CD120B, CD123, CD125, CD134, CD135, CD140A, CD141, CD154, CD155, CD160, CD163,CD172A, XCR1, CD203C, CD204, CD206, CD207 CD226, CD244, CD267, CD268, CD269, CD355, CD358, NKG2A, NKG2B, NKG2C, NKG2D, NKG2E, NKG2F, NKG2H, KIR2DL1, KIR2DL2, KIR2DL3, KIR2DL5A, KIR2DL5B, KIR3DL1, KIR3DL2, KIR3DL3, KIR3DL4, KIR2DS1, KIR2DS2, KIR2DS3, KIR2DS4, KIR2DS5, DAP12, KIR3DS, NKP44, NKP46, TCR, BCR, INTEGRINS, FCβεRI, MHC-I, MHC-II, IL-1R, IL-2Rα, IL-2Rβ, IL-2Rγ, IL-3Rα, CSF2RB, IL-4R, IL-5Rα, CSF2RB, IL-6Rα, GP130, IL-7Rα, IL-9R, IL-12Rβ1, IL-12Rβ2, IL-13Rα1, IL-13Rα2, IL-15Rα, IL-21R, IL23R, IL-27Rα, IL-31Rα, OSMR, CSF-1R, CELLIL-15, IL-10Rα, IL-10R3, IL-20Rα, IL-20Rβ, IL-22Rα1, IL-22Rα2, IL-22Rβ, IL-28RA, PD-1, PD-1H, BTLA, CTLA-4, PD-L1, PD-L2, 2B4, B7-1, B7-2, B7-H1, B7-H4, B7-DC, DR3, LIGHT, LAIR, LTa1β2, LTβR, TIM-1, TIM-3, TIM-4, TIGIT, LAG-3, ICOS, ICOS-L, SLAM, SLAMF2, OX-40, OX-40L, GITR, GITRL, TL1A, HVEM, 41-BB, 41BB-L, TL-1A, TRAF1, TRAF2, TRAF3, TRAF5, BAFF, BAFF-R, APRIL, TRAIL, RANK, AITR, TRAMP, CCR1, CCR2, CCR3, CCR4, CCR5, CCR6, CCR7, CCR8, CCR9, CCR10, CCR11, CXCR1, CXCR2, CXCR3, CXCR4, CXCR5, CXCR6, CXCR7, CLECL9A, DC-SIGN, IGSF4A, SIGLEC, EGFR, PDGFR, VEGFR, FAP, α-SMA, VIMENTIN, LAMININ, FAS, FAS-L, F_{C}, ICAM-1, ICAM-2, ICAM-3, ICAM-4, ICAM-5, PECAM-1, MICA, MICB, UL16, ULBP1, ULBP2, ILBP3, ULBP4, ULBP5, ULBP6, MULT1, RAE1 α, β, γ, δ, AND ε, A₁R, A_{2A}R, A_{2B}R, AND A₃R, H60A, H60B, AND H60C. In various embodiments, the integrins are selected from the group consisting α1, α2, αIIb, α3, α4, α5, α6, α7, α8, α9, α10, α11, αD, αE, αL, αM, αV, αX, β1, β2, β3, β4, β5, β6, β7, β8, and/or combinations thereof. In various embodiments, TCR is selected from the group consisting of α, β, γ, δ, ε, and ζ TCR. Several methods have been described in the literature for assaying of cell-surface protein expression, including Flow Cytometry and Mass Cytometry (CyTOF). The presence or abundance of one or more of these cell-surface proteins indicates that the patient is responsive to treatment with the method disclosed herein.

In various embodiments, administering negatively charged particles in a subject in need thereof increases the levels of tumor inhibiting, anti-tumor, and/or pro-inflammatory proteins in one or more blood and/or tumor samples collected from the subject. In various embodiments, tumor inhibiting, anti-tumor, and/or pro-inflammatory proteins are selected from the group consisting of IL-1α, IL-1β, IL-2, IL-3, IL-4, IL-5, IL-6, IL-7, IL-8, IL-9, IL-10, IL-11, IL-12, IL-13, IL-14, IL-15, IL-16, IL-17, IL-17, IL-18, IL-20, IL-21, IL-22, IL-23, IL-24, IL-25, IL-26, IL-27, IL-28, IL-29, IL-30, IL-31, IL-32, IL-33, IL-35, IL-36, cell-surface IL-15, CXCL2 (MCP-1), CXCL3 (MIP-1α), CXCL4 (MIP-1β), CXCL5 (RANTES), IFN-α, IFN-β, IFN-γ, Granzyme-B, Perforin, and TNF-α. In various embodiments, the levels of anti-tumor, and/or pro-inflammatory proteins are increased by 5-100% (e.g increased by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, 50%, or 100% compared to one or more blood samples collected prior to treatment. In various embodiments, the levels of anti-tumor, and/or pro-inflammatory proteins are increased by 2-100 fold (e.g., increased relative to one or more samples collected prior to treatment by about 2, 5, 10, 15, 20, 25, 30, 35, 40, 45, 50, 55, 60, 65, 70, 75, 80, 85, 90, 95, or 100 fold, inclusive of all values and ranges between these values) compared to one or more blood samples collected from the subject prior to treatment. Several methods have been described in the literature for assaying proteins from blood samples, including western blot, and ELISA.

In various embodiments, administering negatively charged particles in a subject in need thereof reduces the levels of tumor promoting, anti-inflammatory, and/or immune suppressive proteins. In various embodiments, the tumor promoting, anti-inflammatory, and/or immune suppressive proteins are selected from the group consisting CD39, CD79, MMP1, MMP2, MMP3, MMP7, MMP8, MMP9, MMP10, MMP11, MMP12, MMP13, MMP14, MMP15, MMP16, MMP17, MMP18, MMP19, MMP20, MMP21, MMP23A, MMP23B, MMP24, MMP25, MMP26, MMP27 and MMP28, CXCL12, GM-CSF, G-CSF, TGF-β1, TGF-β2, and TGF-β3, arginase, asparaginase, kyneurinase, indoleamine 2,3 dioxygenase (IDO1 and IDO2), tryptophan 2,3 dioxygenase (TDO), myeloperoxidase (MPO), neutrophil elastase (NE), and IL4I1. In various embodiments, the levels of tumor promoting, anti-inflammatory, and/or immune suppressive proteins in one or more blood and/or tumor samples of the subject are decreased by 5-100% (e.g., decreased by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, 50%, or 100% compared to one or more blood and/or tumor samples collected prior to treatment. In various embodiments, the levels of tumor promoting, anti-inflammatory, and/or immune suppressive proteins in one or more blood and/or tumor samples samples of the subject are decreased by 2-100 fold (e.g., decreased relative to one or more samples collected prior to treatment by about 2, 5, 10, 15, 20, 25, 30, 35, 40, 45, 50, 55, 60, 65, 70, 75, 80, 85, 90, 95, or 100 fold, inclusive of all values and ranges between these values) compared to one or more blood samples collected from the subject prior to treatment.

In various embodiments, administering negatively charged particles in a subject in need thereof increases the levels of tumor inhibiting, anti-tumor, and/or pro-inflammatory genes in one or more samples collected from the subject. In one or more embodiments, the expression of tumor inhibiting, anti-tumor, and/or pro-inflammatory genes is increased by 5-100% (e.g increased by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, 50%, or 100% compared to one or more blood and/or tumor samples collected prior to treatment. In various embodiments, the expression of tumor inhibiting, anti-tumor, and/or pro-inflammatory genes is increased by 2-100-fold (e.g., increased by about 2, 5, 10, 15, 20, 25, 30, 35, 40, 45, 50, 55, 60, 65, 70, 75, 80, 85, 90, 95, or 100 fold, inclusive of all values and ranges between these values) compared to one or more blood and/or tumor samples collected from the subject prior to treatment. In various embodiments, administering negatively charged particles in a subject in need thereof decreases the levels of tumor promoting and/or anti-inflammatory genes in one or more samples collected from the subject. In one or more embodiments, the expression of tumor promoting and/or anti-inflammatory genes is decreased by 5-100% (e.g increased by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, 50%, or 100% compared to one or more blood and/or tumor samples collected prior to treatment. In various embodiments, the expression of tumor promoting and/or anti-inflammatory genes is increased by 2-100-fold (e.g., increased by about 2, 5, 10, 15, 20, 25, 30, 35, 40, 45, 50, 55, 60, 65, 70, 75, 80, 85, 90, 95, or 100 fold, inclusive of all values and ranges between these values) compared to one or more blood and/or tumor samples collected from the subject prior to treatment. In various embodiments, the gene expression analysis is performed by PCR, RT-PCR, qRT-PCR, next-generation sequencing (NGS), RNA-seq, ATAC-seq, exome sequencing, Southern Blot, microarray analysis, and/or single-cell sequencing.

In various embodiments, administering the negatively charged particles to a subject in need thereof reduces the levels of neutrophil extracellular traps (NETs) in one or more blood and/or tumor samples collected from the subject. In various embodiments, the levels of NETs in one or more blood and/or tumor samples is decreased by 5-100% (e.g., decreased by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, 50%, or 100% compared to one or more tumor samples collected prior to treatment. In various embodiments, the levels of NETs in one or more blood and/or tumor samples is decreased by 2-100-fold (e.g., decreased relative to one or more samples collected prior to treatment by about 2, 5, 10, 15, 20, 25, 30, 35, 40, 45, 50, 55, 60, 65, 70, 75, 80, 85, 90, 95, or 100 fold, inclusive of all values and ranges between these values) compared to one or more blood and/or tumor samples collected from the subject prior to treatment. Several methods have been described in the literature for assaying NETs from tumor samples, including western blot, ELISA, and flow cytometry.

In various embodiments, administering the negatively charged particles to a subject in need thereof reduces the neutrophil to lymphocyte (NLR) in one or more blood samples from high to moderate, or high to low. In various embodiments, the NLR is reduced to a level between 1-2 (e.g., between 1, 1.1, 1.2, 1.3, 1.4, 1.5, 1.6, 1.7, 1.8, 1.9, and 2 inclusive of all values and ranges between these values).

In various embodiments, administering the negatively charged particles to a subject in need thereof alters the levels of one or more metabolites associated with tumor growth and progression. In various embodiments, administering the negatively charged particles to a subject in need thereof increases the levels of one or more anti-tumor metabolites. In various embodiments, the anti-metabolite is an inflammatory metabolite. In various embodiments, examples of inflammatory metabolites include acids, lipids, sugars, amino acids, lactate, trimethylamine N-oxide, O-acetyl creatine, L-carnitine, choline, succinate, glutamine, fatty acids, cholesterol, 3-hydroxybutyrate, 3′-sialyllactose, arachidonic acid, prostaglandin (G2 and H2), PGD2, PGE2, PGF2a, PGI2, TXA2, leukotrienes (A4, B4, C4, D4, E4), lipoxin A4, and lipoxin B4. In various embodiments, the levels of anti-tumor metabolites in one or more blood and/or tumor samples is increased by 5-100% (e.g., decreased by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, 50%, or 100% compared to one or more tumor samples collected prior to treatment. In various embodiments, the levels of anti-tumor metabolites in one or more blood and/or tumor samples is increased by 2-100-fold (e.g., decreased relative to one or more samples collected prior to treatment by about 2, 5, 10, 15, 20, 25, 30, 35, 40, 45, 50, 55, 60, 65, 70, 75, 80, 85, 90, 95, or 100 fold, inclusive of all values and ranges between these values) compared to one or more blood and/or tumor samples collected from the subject prior to treatment.

In various embodiments, administering the negatively charged particles to a subject in need thereof reduces the levels of one or more pro-tumor metabolites. In various embodiments, the pro-tumor metabolite is an anti-inflammatory metabolite. In various embodiments, examples of anti-inflammatory metabolites include kynurenine, 3-hydroxy kynurenine, 2-amino-3-carboxymuconic 6-semialdehyde, picolinic acid, anthranilic acid, 3-hydroxylanthranilic acid, glutaryl co-A, NAD+, quinolinic acid, arginine, butyrate, and adenosine. In various embodiments, the levels of pro-tumor metabolites in one or more blood and/or tumor samples is decreased by 5-100% (e.g., decreased by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, 50%, or 100% compared to one or more tumor samples collected prior to treatment. In various embodiments, the levels of pro-tumor metabolites in one or more blood and/or tumor samples is decreased by 2-100-fold (e.g., decreased relative to one or more samples collected prior to treatment by about 2, 5, 10, 15, 20, 25, 30, 35, 40, 45, 50, 55, 60, 65, 70, 75, 80, 85, 90, 95, or 100 fold, inclusive of all values and ranges between these values) compared to one or more blood and/or tumor samples collected from the subject prior to treatment

A list of human metabolites that can be assayed from a biological sample can be found in the literature including in (Psychogios et al., 2011), (Wishart et al., HMDB: the Human Metabolome Database. Nucleic Acids Res. 2007 January; 35(Database issue):D521-6, 2007), and the Human Metabalome Database (HMDB) and are incorporated herein by reference.

In various embodiments, the therapeutic efficacy of negatively charged particles administered to a subject with cancer is determined based on successful demonstration of at least one of the following parameters:
Decrease in tumor burden as determined by RECIST.
Decrease in in number of circulating tumor cells (CTCs) in blood after treatment and/or during treatment with negatively charged particles when compared to one or more samples collected prior to treatment with negatively charged particles.
Increase in the frequency of T cells and NK cells in blood and/or tumor after treatment and/or during treatment with negatively charged particles when compared to one or more samples collected prior to treatment with negatively charged particles.
Increase in the frequency of activated t cells and nk cells in blood and/or tumor after treatment and/or during treatment with negatively charged particles when compared to one or more samples collected prior to treatment with negatively charged particles.
Increase in IL-15 gene expression levels in APCs in blood and/or tumor after treatment and/or during treatment with negatively charged particles when compared to one or more samples collected prior to treatment with negatively charged particles.
Decrease in levels of neutrophil extracellular traps (NETs) in blood after treatment and/or during treatment with negatively charged particles when compared to one or more samples collected prior to treatment with negatively charged particles.
Decrease in the neutrophil to lymphocyte ratio (NLR) in blood after treatment and/or during treatment with negatively charged particles when compared to one or more samples collected prior to treatment with negatively charged particles.
Increase in the levels of one or more anti-tumor cytokines and chemokines in blood and/or tumor after treatment and/or during treatment with negatively charged particles when compared to one or more samples collected prior to treatment with negatively charged particles.
Increase in the frequency of activated APCs in blood and/or tumor after treatment and/or during treatment with negatively charged particles when compared to one or more samples collected prior to treatment with negatively charged particles.

### Administration and Dosing

Contemplated herein are methods comprising administering a composition comprising negatively charged surface functionalized particles as described herein alone or in combination with one or more cancer therapeutic to treat a subject suffering from cancer.

Methods of the disclosure are performed using any medically accepted means for introducing a therapeutic directly or indirectly into a mammalian subject, including but not limited to injections, oral ingestion, intranasal, topical, transdermal, parenteral, inhalation spray, vaginal, or rectal administration. The term parenteral as used herein includes subcutaneous, intravenous, intramuscular, intraarticular, intraperitoneal, intrathecal and intracisternal injections, as well as catheter or infusion techniques. In various embodiments, the particle is administered intravenously, but may be administered through other routes of administration such as, but not limited to: intradermal, subcutaneous, epictuaneous, oral, intra-articular, and intrathecal.

In various embodiments, the compositions are administered at the site of the tumor. negatively charged particles are administered at a dose of about 0.1 mg/kg to 15 mg/kg. In various embodiments, the negatively charged particles are administered at a dose of about 0.5 mg/kg to 10 mg/kg, from about 1 mg/kg to 8 mg/kg, from about 1.5 mg/kg to 15 mg/kg, from about 2 mg/kg to 15 mg/kg, from about 2 mg/kg to 10 mg/kg, from about 3 mg/kg to 10 mg/kg, from about 4 mg/kg to 10 mg/kg, from about 4 mg/kg to 15 mg/kg, from about 5 mg/kg to 15 mg/kg, or from about 2 mg/kg to 8 mg/kg. In various embodiments, negatively charged particles are administered at a dose of about 0.1 mg/kg, 0.25 mg/kg, 0.5 mg/kg, 1.0 mg/kg, 2.0 mg/kg, 3.0 mg/kg, 4.0 mg/kg, 5 mg/kg, 6 mg/kg, 8.0 mg/kg, 10 mg/kg, 12 mg/kg, or 15 mg/kg, including any values or ranges therebetween. In various embodiments, negatively charged particles are administered at a dose of about 10 mg, 25 mg, 50 mg, 75 mg, 100 mg, 125 mg, 150 mg, 175 mg, 200 mg, 225 mg, 250 mg, 275 mg, 300 mg, 325 mg, 350 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg, 800 mg, 850 mg, 875 mg, 900 mg, 950 mg, 975 mg, or 1000 mg, including any values or ranges therebetween. These dose levels may be administered as a single dose or multiple doses.

In various embodiments, negatively charged particles are administered at a concentration of about 0.05 mg/mL and about 50 mg/mL. In various embodiments, negatively charged particles are administered at a concentration of about 0.1 mg/mL, 0.5 mg/mL, 1 mg/mL, 2 mg/mL, 3 mg/mL, 4 mg/mL, 5 mg/mL, 6 mg/mL, 7 mg/mL, 8 mg/mL, 9 mg/mL, 10 mg/mL, 11 mg/mL, 12.5 mg/mL, 15 mg/mL, 17.5 mg/mL, 20 mg/mL, 25 mg/mL, 30 mg/mL, 40 mg/mL, or 50 mg/mL, including any values or ranges therebetween. In various embodiments, negatively charged particles are administered via intravenous infusion lasting about 1, 2, 3, 4, 5, 6, 7, or 8 hours.

In various embodiments, the negatively charged particles are administered once daily, twice daily, three times per day, seven times per week, six times per week, five times per week, four times per week, three times per week, twice weekly, once weekly, once every two weeks, once every three weeks, once every 4 weeks, once every two months, once every three months, once every 6 months or once per year. In various embodiments, the negatively charged surface functionalized particle and/or the cancer therapeutic is administered for 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, or 52 weeks, or more.

In various embodiments, the negatively charged particles are administered in treatment cycles. In various embodiments, one treatment cycle consists of 1, 2, 3, 4, 5, 6, or 7 doses administered weekly. In various embodiments, the subject is administered 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, or 52 treatment cycles of negatively charged particles.

The disclosure further contemplates a sterile pharmaceutical composition comprising a particle as described herein, a cancer therapeutic and a pharmaceutically acceptable carrier.

The disclosure further contemplates a sterile pharmaceutical composition comprising a separate a particle as described herein and a pharmaceutically acceptable carrier.

The disclosure further contemplates a sterile pharmaceutical composition comprising a separate cancer therapeutic and a pharmaceutically acceptable carrier.

Syringes, e.g., single use or pre-filled syringes, sterile sealed containers, e.g., vials, bottle, vessel, and/or kits or packages comprising any of the foregoing antibodies or compositions, optionally with suitable instructions for use, are also contemplated.

### Combination Therapy

It is contemplated that the particles described herein are administered in combination with a cancer therapeutic to treat cancer or a proliferative disorder. In various embodiments, the cancer therapeutic is a chemotherapeutic, a biologic agent, a cell-based therapy, a hormone therapy, an antibody-drug conjugate, oncolytic virus, or a cancer vaccine.

Hormone therapies include Tamoxifen for breast cancer, Zoladex for breast cancer and prostate cancer, Aromatase inhibitors (e.g anastrazole, letrozole, exemestane). Antibody drug conjugates include Brentuximab vedotin for lymphomas. (anti-CD30 mAB+monomethyl auristatin E), Ado-trastuzumab entansine for breast cancers. (anti-Her2/Neu+maytansinoid) and Inotuzumab Ozagamicin for ALL (anti-CD22+calicheamicin). Oncolytic viruses include Imlygic (Amgen®). Cancer vaccines include Sipuleucel-T for prostate cancer. Several cancer vaccines are in development and include, but are not limited to, proteins, polypeptides, and nucleic acid vaccines.

In various embodiments, the cancer therapeutic is a chemotherapeutic selected from the group consisting of growth inhibitors, a cytotoxic agent, DNA-replication inhibitors, kinase inhibitors, signaling cascade inhibitors, angiogenesis inhibitors, metabolic inhibitors, amino acid synthesis inhibitors, selective inhibitors of oncogenic proteins, inhibitors of metastasis, inhibitors of anti-apoptosis factors, apoptosis inducers, nucleoside signaling inhibitors, enzyme inhibitors and DNA-damaging agents.

A cytotoxic agent refers to a substance that inhibits or prevents the function of cells and/or causes destruction of cells. The term is intended to include radioactive isotopes (e.g., i131, i125, y90 and re186), chemotherapeutic agents, and toxins such as enzymatically active toxins of bacterial, fungal, plant or animal origin or synthetic toxins, or fragments thereof. A non-cytotoxic agent refers to a substance that does not inhibit or prevent the function of cells and/or does not cause destruction of cells. A non-cytotoxic agent may include an agent that can be activated to be cytotoxic.

Chemotherapeutic agents contemplated for use in the methods of the present disclosure include, but are not limited to those listed in Table 1:

TABLE 1
chemotherapeutic agents

| Alkylating agents | Natural products |
|---|---|
| Nitrogen mustards | Antimitotic drugs |
| mechlorethamine | Taxanes |
| cyclophosphamide | paclitaxel |
| ifosfamide | Vinca alkaloids |
| melphalan | vinblastine (VLB) |
| chlorambucil | vincristine |
| bendamustine | vinorelbine |
| trabectedin | Taxotere ® (docetaxel) |
| temozolomide | estramustine |
| Nitrosoureas | estramustine phosphate |
| carmustine (BCNU) | Epipodophylotoxins |
| lomustine (CCNU) | etoposide |
| semustine (methyl-CCNU) | teniposide |
| streptozocin | Antibiotics |
| Ethylenimine/Methyl-melamine | actimomycin D |
| thriethylenemelamine (TEM) | daunomycin (rubido-mycin) |
| triethylene thiophosphoramide | doxorubicin (adria-mycin) |
| (thiotepa) | mitoxantroneidarubicin |
| hexamethylmelamine | bleomycin |
| (HMM, altretamine) | splicamycin (mithramycin) |
| Alkyl sulfonates | mitomycinC |
| busulfan | dactinomycin |
| Triazines | aphidicolin |
| dacarbazine (DTIC) | valrubicin |
| Antimetabolites | epirubicin |
| Folic Acid analogs | Enzymes |
| methotrexate | L-asparaginase |
| Trimetrexate | L-arginase |
| Pemetrexed | Radiosensitizers |
| (Multi-targeted antifolate) | metronidazole |
| Pyrimidine analogs | misonidazole |
| 5-fluorouracil | desmethylmisonidazole |
| capecitabine | pimonidazole |
| fluorodeoxyuridine | etanidazole |
| gemcitabine | nimorazole |
| cytosine arabinoside | RSU 1069 |
| (AraC, cytarabine) | EO9 |
| 5-azacytidine | RB 6145 |
| decitabine | SR4233 |
| 2,2′-difluorodeoxy-cytidine | nicotinamide |
| Purine analogs | 5-bromodeozyuridine |
| 6-mercaptopurine | 5-iododeoxyuridine |
| 6-thioguanine | bromodeoxycytidine |
| azathioprine | Miscellaneous agents |
| 2′-deoxycoformycin | Platinium coordination complexes |
| (pentostatin) | cisplatin |
| erythrohydroxynonyl-adenine (EHNA) | Carboplatin |
| fludarabine phosphate | oxaliplatin |
| 2-chlorodeoxyadenosine | Anthracenedione |
| (cladribine, 2-CdA) | mitoxantrone |
| nelarabine | Substituted urea |
| clofarabine | hydroxyurea |
| Type I Topoisomerase Inhibitors | Methylhydrazine derivatives |
| camptothecin | N-methylhydrazine (MIH) |
| topotecan | procarbazine |
| irinotecan | Adrenocortical suppressant |
| Biological response modifiers | mitotane (o, p′- DDD) |
| G-CSF | ainoglutethimide |
| GM-CSF | Cytokines |
| Differentiation Agents | interferon (α, β, γ) |
| retinoic acid derivatives | interleukin-2 |
| Hormones and antagonists | Photosensitizers |
| Adrenocorticosteroids/antagonists | hematoporphyrin derivatives |
| prednisone and equivalents | Photofrin ® |
| dexamethasone | benzoporphyrin derivatives |
| ainoglutethimide | Npe6 |
| Progestins | tin etioporphyrin (SnET2) |
| hydroxyprogesterone caproate | pheoboride-a |
| medroxyprogesterone acetate | bacteriochlorophyll-a |
| megestrol acetate | naphthalocyanines |
| Estrogens | phthalocyanines |
| diethylstilbestrol | zinc phthalocyanines |
| ethynyl estradiol/ equivalents | Radiation |
| Antiestrogen | X-ray |
| tamoxifen | ultraviolet light |
| Androgens | gamma radiation |
| testosterone propionate | visible light |
| fluoxymesterone/equivalents | infrared radiation |
| Antiandrogens | microwave radiation |
| flutamide | cobalt |
| gonadotropin-releasing | linear accelerator |
| hormone analogs | neutron beam |
| leuprolide | betatron |
| Nonsteroidal antiandrogens | spray radiation |
| flutamide | stereotatic radiation therapy |
| | gamma knife |
| | Proteasome inhibitors |
| | bortezomib |

It is also contemplated that the cancer therapeutic comprises one or more biologic agents, such as cytokines, angiogenesis inhibitors, immune checkpoint modulators and monoclonal antibodies.

Cytokines include interferons (IFN) and interleukins (ILs), such as IFN-alpha, IFN-beta, IFN-gamma, IL-1, IL-2, IL-4, IL-5, IL-6, IL-7, IL-10, IL-11, IL-12, IL-13, IL-15, IL-17, IL-18, IL-21, members of the transforming growth factor beta superfamily, including TGF-β1, TGF-β2 and TGF-β3, tumor necrosis factor alpha, Granulocyte colony-stimulating factor (G-CSF), and Granulocyte macrophage colony-stimulating factor (GM-CSF).

In various embodiments, the cancer therapeutic comprises an enzyme. In various embodiments, the cancer therapeutic comprises an enzyme that targets T-cells, B-cells, APCs, monocytes, MDSCs, TAMs, neutrophils, other monocyte-derived cells, tumor-associated stroma, cancer stem cells, mesenchymal stem cells, extracellular matrix, and amino acids. In various embodiments, the cancer therapeutic comprises an enzyme selected from the group comprising asparaginase, kynurininase, L-arginine deiminase, L-methionine-γ-lyase, one or more amino acid degrading enzymes, and one or more nucleoside degrading enzymes.

Biologic agents such as immune checkpoint modulators target PD1, PD-L1, CTLA-4, TIMP-3, LAG-3 and/or TIGIT (T cell immunoreceptor with Ig and ITIM domains). In various embodiments, the immune checkpoint modulators are antibodies specific for PD-1, PD-L1, or CTLA-4. Antibodies specific for checkpoint proteins include ipilimumab (YERVOY®, Bristol-Myers Squibb Company), and tremelimumab that bind CTLA-4; antibodies to PD-1 such as Pembrolizumab (KEYTRUDA®, Merck Sharp & Dohme Corp), dostarlimab (Jemperli, GSK) and nivolumab (OPDIVO®, Bristol-Myers Squibb); and antibodies that target PD-L1 such as Atezolizumab (TECENTRIQ®), Avelumab (BAVENCIO®), and Durvalumab (IMIFINZI®) (approved for treatment of urothelial carcinoma and non-small cell lung carcinoma), Cemiplimab (LIBTAYO®) (approved for cutaneous squamous cell carcinoma).

In various embodiments the monoclonal antibodies are mono-specific, bi-specific, tri-specific or bispecific T-cell engaging (BiTE) antibodies.

In various embodiments the monoclonal antibodies are immune cell co-stimulatory molecule agonists that induce an anti-tumor immune response. Exemplary co-stimulatory molecules include, but are not limited to, ICOS (Inducible T cell Co-stimulator) (CD278), OX40 (CD134), GITR (Glucocorticoid-induced Tumor Necrosis Factor Receptor), CD40 and CD27.

In various embodiments, monoclonal antibodies useful in the methods are selected from the group comprising Alemtuzumab, Bevacizumab, Brentuximab, Cetuximab, Denosumab, Ibritumomab, Trastuzumab, Panitumumab, Pertuzumab, and Rituximab. In various embodiments, monoclonal antibodies useful in the methods target receptor tyrosine kinase, EGFR, VEGF, VEGFR, PDGF, PDGFR, TGF-β, TGF-β-LAP, SIRP-α, CD47, CD39, CD73, and fibroblast activating protein (FAP).

Biologic agents include monoclonal antibodies that are mono-specific, bi-specific, tri-specific or bispecific T-cell engagers (BiTE). Monoclonal antibodies useful in the treatment of cancer include bevacizumab (AVASTIN®, Genentech), an antibody to VEGF-A; erlotinib (TARCEVA®, Genentech and OSI Pharmaceuticals), a tyrosine kinase inhibitor which acts on EGFR, dasatinib (SPRYCEL®, Bristol-Myers Squibb Company), an oral Bcr-Abl tyrosone kinase inhibitor; IL-21; pegylated IFN-α2b; axitinib (INLYTA®, Pfizer, Inc.), a tyrosine kinase inhibitor; and trametinib (MEKINIST®, GlaxoSmithKline), a MEK inhibitor (Philips and Atkins, Int Immunol., 27(1):39-46 (2015) which is incorporated herein by reference). Bispecific antibodies useful to treat cancer are described in Krishnamurthy et al., (Pharmacol Ther. 2018 May;185:122-134), and Yu et al., (J. Hematol Oncol 2017, 10:155), including Blinatumomab and catumaxomab.

The method also provides that the cancer therapeutic comprises one or more cell-based therapies including adoptive cell transfer, tumor-infiltrating leukocyte therapy, chimeric antigen receptor T-cell (CAR-T) therapy, NK-cell therapy, invariant NKT cell therapy and stem cell therapy.

In various embodiments the cell-based therapy is the adoptive transfer of autologous patient-derived cells. In various embodiments the cell-based therapy is the adoptive transfer of allogenic donor-derived cells.

In various embodiments, the cell-based therapy is the transfer of universal donor-derived or induced pluripotent stem cell-derived cells that are not patient specific and amenable to long-term storage. Such therapies are also referred to as ‘off-the-shelf’ therapies.

In various embodiments, the cancer therapeutic is a hormone therapy. In various embodiments, the cancer therapeutic comprises one or more antibody-drug conjugates. In various embodiments, the cancer therapeutic comprises one or more cancer vaccines. In various embodiments, the cancer vaccine is a protein, polypeptide, and/or nucleic acid vaccine.

In various embodiments, the cancer therapeutic is an immunotherapy selected from the group comprising oncolytic virus, bacteria, oncolytic bacteria or other bacterial consortia, Bacillus Calmette-Guerin (BCG), a microbiome modulator, and/or a toll-like receptor (TLR) agonist. In various embodiments, the TLR agonist is a TLR3, TLR4, TLR5, TLR7, TLR8, TLR9, TLR10, TLR11, TLR12, and/or TLR13 agonist. In various embodiments, the TLR agonist is derived from virus, bacteria and/or made synthetically. In various embodiments, the immunotherapy is a STING pathway modulator.

In various embodiments, the cancer therapeutic comprises a viral or a bacterial vector. In various embodiments, the viral vector is selected from the group comprising adenovirus, adeno-associated virus (AAV), herpes simplex virus, lentivirus, retrovirus, alphavirus, flavivirus, rhabdovirus, measles virus, Newcastle disease virus, poxvirus, vaccinia virus, modified Ankara virus, vesicular stomatitis virus, picornavirus, tobacco mosaic virus, potato virus x, comovirus or cucumber mosaic virus. In various embodiments, the virus is an oncolytic virus. In various embodiments the virus is a chimeric virus, a synthetic virus, a mosaic virus or a pseudotyped virus.

It is contemplated that the particle and the cancer therapeutic can be given concurrently, simultaneously, or sequentially. Concurrent administration of two therapeutic agents does not require that the agents be administered at the same time or by the same route, as long as there is an overlap in the time period during which the agents are exerting their therapeutic effect. Simultaneous or sequential administration is contemplated, as is administration on different days or weeks.

It is contemplated that the particle and the cancer therapeutic may be given simultaneously, in the same formulation. It is further contemplated that the agents are administered in a separate formulation and administered concurrently, with concurrently referring to agents given within 30 minutes of each other.

In another aspect, the cancer therapeutic is administered prior to administration of the particle composition. Prior administration refers to administration of the cancer therapeutic within the range of one week prior to treatment with the particle, up to 30 minutes before administration of the particle. It is further contemplated that the cancer therapeutic is administered subsequent to administration of the particle composition. Subsequent administration is meant to describe administration from 30 minutes after particle treatment up to one week after administration.

In various embodiments, the particle and/or the cancer therapeutic is administered once daily, twice daily, three times per day, seven times per week, six times per week, five times per week, four times per week, three times per week, twice weekly, once weekly, once every two weeks, once every three weeks, once every 4 weeks, once every two months, once every three months, once every 6 months or once per year.

In various embodiments, the particle and/or the cancer therapeutic is administered intravenously, orally, nasally, intramuscularly, ocularly, transdermally, or subcutaneously.

In various embodiments, the subject is a mammal. In various embodiments, the subject is human.

### Kits

As an additional aspect, the disclosure includes kits which comprise one or more compounds or compositions packaged in a manner which facilitates their use to practice methods of the disclosure. In one embodiment, such a kit includes a compound or composition described herein (e.g., a particle alone or in combination with a cancer therapeutic, or compositions thereof), packaged in a container such as a sealed bottle or vessel, with a label affixed to the container or included in the package that describes use of the compound or composition in practicing the method. Preferably, the compound or composition is packaged in a unit dosage form. The kit may further include a device suitable for administering the composition according to a specific route of administration or for practicing a screening assay. Preferably, the kit contains a label that describes use of the inhibitor compositions.

### Methods of Manufacture

In various embodiments, the present disclosure also provides a process for manufacturing a composition comprising negatively charged particles. In various embodiments, the method comprises: (a) generating a primary emulsion by mixing an aqueous solution with an oil phase including a polymer; (b) mixing the primary emulsion with a solution including one or more surfactants and/or stabilizers to form a secondary emulsion; (c) hardening the secondary emulsion by evaporation to remove the solvent resulting in hardened polymeric nanoparticles (d) filtering, washing, and concentrating the nanoparticles; and (e) freeze drying the nanoparticles. In various embodiments, the primary emulsion of step (a) is a water-in-oil emulsion. In various embodiments, the secondary emulsion of step (b) is an oil-in-water emulsion.

In embodiments, the method comprises: (a) generating a primary emulsion by mixing an aqueous solution with an oil phase including a polymer; (b) mixing the primary emulsion with a solution including one or more surfactants and/or stabilizers to form a secondary emulsion; (c) removing the solvent to form crude negatively charged particles; and (d) filtering the crude particles to form a composition comprising negatively charged particles, wherein the composition comprises less than 0.5% wt. (e.g., less than 0.4% wt., less than 0.3% wt., less than 0.2% wt., less than 0.1% wt., less than 0.05% wt., less than 0.01% wt.) of the one or more surfactants and less than 1% wt. (e.g., less than 0.9% wt., less than 0.8% wt., less than 0.7% wt., less than 0.6% wt., less than 0.5% wt., 0.4% wt., less than 0.3% wt., less than 0.2% wt., less than 0.1% wt., less than 0.05% wt., less than 0.01% wt.) of the one or more stabilizer. In embodiments, the crude particles were filtered through a filter with a pore size of about 1 μm, about 2 μm, about 5 μm, about 10 μm, about 20 μm, about 30 μm, about 40 μm, or about 50 μm, including any values or ranges therebetween. In embodiments, the filter has a pore size of about 10 μm, about 20 μm.

In various embodiments, the process of manufacturing is designed to obtain negatively charged particles having an average diameter of about 0.1 μm to about 10 μm, or about 0.1 μm to about 5 μm, or about 0.1 μm to about 3 μm, or about 0.3 μm to about 5 μm, or about 0.3 μm to about 3 μm, or about 0.3 μm to about 1 μm, including all values and ranges therein. In various embodiments, the process for manufacturing is designed to obtain negatively charged particles having a negative zeta potential of about −100 mV to about −10 mV, or about −100 mV to about −25 mV, or about −100 to about −30 mV, or about −80 mV to about −30 mV, or about −75 mV to about −30 mV, or about −70 mV to about −30 mV, or from about −60 mV to about −30 mV, or about −75 to about −35 mV, or about −70 to about −25 mV, including all values and ranges therein.

In various embodiments, the aqueous solution of step (a) includes a solvent. In various embodiments, the solvent is an organic solvent. In various embodiments, the solvent is an inorganic solvent. In various embodiments, the organic solvent is dichloromethane, acetone, ethanol, methylene chloride, dimethyl sulfoxide (DMSO), ethyl acetate, dimethylformamide, tetrahydrofuran, chloroform, and acetic acid. In various embodiments, the inorganic solvent is water, ammonia, sulphuric acid, carbon disulphide, bromine trifluoride, phosphorous oxychloride, hydrogen fluoride, and sulphur dioxide. In various embodiments, the solvent in the aqueous solution is at a concentration of 1, 2, 3, 5, 10, 15, 20, 25, 30, 35, 40, 45, or 50% (v/v). In various embodiments, the solvent in the aqueous solution is at a concentration of 0.1, 0.2, 0.3, 0.4, 0.5, 0.6, 0.7, 0.8, 0.9, 1.0, 1.5, 2, 2.5, 3, 3.5, 4, 4.5, 5, 5.5, 6, 6.5, 7.0, 7.5, 8.0, 8.5, 9.0, or 10.0 mM. In various embodiments, the solvent in the aqueous solution is at a concentration of 0.1, 0.2, 0.3, 0.4, 0.5, 0.6, 0.7, 0.8, 0.9, 1.0, 1.5, 2, 2.5, 3, 3.5, 4, 4.5, 5, 5.5, 6, 6.5, 7.0, 7.5, 8.0, 8.5, 9.0, or 10.0 M. In various embodiments, the selection of the solvent(s) in the process for manufacturing is designed to obtain negatively charged particles having a diameter of about 0.1 μm to about 10 μm, or about 0.1 μm to about 5 μm, or about 0.1 μm to about 3 μm, or about 0.3 μm to about 5 μm, or about 0.3 μm to about 3 μm, or about 0.3 μm to about 1 μm, including all values and ranges therein. In various embodiments, the the selection of the solvent(s) in the process for manufacturing is designed to obtain negatively charged particles having a negative zeta potential of about −100 mV to about −10 mV, or about −100 mV to about −25 mV, or about −100 to about −30 mV, or about −80 mV to about −30 mV, or of about −75 mV to about −30 mV, or about −70 mV to about −30 mV, or from about −60 mV to about −30 mV, or about −75 to about −35 mV, or about −70 to about −25 mV, including all values and ranges therein.

In various embodiments, the surfactant and/or stabilizer solution of step (b) includes a solvent. In various embodiments, the solvent is an organic solvent. In various embodiments, the solvent is an inorganic solvent. In various embodiments, the organic solvent is dichloromethane, acetone, ethanol, methylene chloride, dimethyl sulfoxide (DMSO), ethyl acetate, dimethylformamide, tetrahydrofuran, chloroform, and acetic acid. In various embodiments, the inorganic solvent is water, ammonia, sulphuric acid, carbon disulphide, bromine trifluoride, phosphorous oxychloride, hydrogen fluoride, and sulphur dioxide. In various embodiments, the solvent in the aqueous solution is at a concentration of 1, 2, 3, 5, 10, 15, 20, 25, 30, 35, 40, 45, or 50% (v/v). In various embodiments, the solvent in the aqueous solution is at a concentration of 0.1, 0.2, 0.3, 0.4, 0.5, 0.6, 0.7, 0.8, 0.9, 1.0, 1.5, 2, 2.5, 3, 3.5, 4, 4.5, 5, 5.5, 6, 6.5, 7.0, 7.5, 8.0, 8.5, 9.0, or 10.0 mM. In various embodiments, the solvent in the aqueous solution is at a concentration of 0.1, 0.2, 0.3, 0.4, 0.5, 0.6, 0.7, 0.8, 0.9, 1.0, 1.5, 2, 2.5, 3, 3.5, 4, 4.5, 5, 5.5, 6, 6.5, 7.0, 7.5, 8.0, 8.5, 9.0, or 10.0 M. In various embodiments, the solvent in the solution of step (a) and step (b) are the same. In various embodiments, the solvents in the solution of step (a) and step (b) are different.

In various embodiments, the polymer in step (a) is a biodegradable polymer. In various embodiments, the biodegradable polymer is polyglycolic acid (PGA), polylactic acid (PLA), polysebacic acid (PSA), poly(lactic-co-glycolic) (PLGA), poly(lactic-co-sebacic) acid (PLSA), poly(glycolic-co-sebacic) acid (PGSA), polypropylene sulfide, poly(caprolactone), chitosan, a polysaccharide, or a lipid. In various embodiments, the polymer is a co-polymer. In various embodiments, the co-polymer has varying molar ratios of constituent polymers. In various embodiments, the molar ratio is 25:75, 30:70, 35:65, 40:60, 45:55, 50:50, 55:45, 60:40, 65:35, 70:30, 75:25, 80:20, 81:19, 82:18, 83:17, 84:16, 85:15, 86:14, 87:13, 88:12, 89:11, 90:10, 91:9, 92:8, 93:7, 94:6, 95:5, 96:4, 97:3, 98:2, 99:1, or 100:0.

In various embodiments, the polymer in step (a) is PLGA. In various embodiments, the molar ratio of co-polymers of PLGA are 25:75, 30:70, 35:65, 40:60, 45:55, 50:50, 55:45, 60:40, 65:35, 70:30, 75:25, 80:20, 81:19, 82:18, 83:17, 84:16, 85:15, 86:14, 87:13, 88:12, 89:11, 90:10, 91:9, 92:8, 93:7, 94:6, 95:5, 96:4, 97:3, 98:2, 99:1, or 100:0. In various embodiments, the PLGA has a high molecular weight. In various embodiments, the PLGA has a low molecular weight. In various embodiments, the PLGA has a molecular weight of about 10 to 10,000 kDa (e.g., between 10, 20, 30, 40, 50, 60, 70, 80, 90, 100, 500, 1000, 2000, 3000, 4000, 5000, 6000, 7000, 8000, 9000, or 10000 kDa including all values lying within this range). In various embodiments, the amount of PLGA in the solution of step (a) is about 0.05 to 100% (e.g., between 0.05, 0.1, 0.2, 0.3, 0.4, 0.5, 0.6, 0.7, 0.8, 0.9, 1, 2, 3, 4, 5, 6, 7, 8, 9, 10, 20, 30, 40, 50, 60, 70, 80, 90, or 100% including all values lying within this range) by weight.

In various embodiments, the surfactant and/or stabilizer used in step (b) is anionic, cationic, or nonionic. In various embodiments, the surfactant and/or stabilizer is a poloxamer, a polyamine, PEG, Tween-80, gelatin, dextran, pluronic L-63, pluronic F-68, pluronic 188, pluronic F-127, PVA, PAA, methylcellulose, lecithin, DMAB, PEMA, vitamin E TPGS (D-a-tocopheryl polyethylene glycol 1000 succinate), hyaluronic acid, poly amino acids (e.g., polymers of lysine, arginine, aspartic acid, glutamic acid, serine, threonine, tyrosine and cysteine, or their enantiomers), methylcellulose, hydroxyethylcellulose, hydroxyprolylcellulose, hydroxypropylmethylcellulose, gelatin, a carbomer, or a sulfate polymer (e.g., heparin sulfate, chondroitin sulfate, fucoidan, ulvan, and carrageenan). In various embodiments, the amount of surfactant and/or stabilizer present in the solution in step (b) is about 0.0005% to 100% (e.g., between 0.0005, 0.001, 0.005, 0.01, 0.05, 0.1, 0.2, 0.3, 0.4, 0.5, 0.6, 0.7, 0.8, 0.9, 1, 2, 3, 4, 5, 6, 7, 8, 9, 10, 20, 30, 40, 50, 60, 70, 80, 90, or 100% including all values lying within this range) by weight or volume. In various embodiments, the surfactant and/or stabilizer have a molecular weight of about 0.1 kDa to 10,000 kDa (e.g., between 0.1, 0.2, 0.3, 0.4, 0.5, 0.6, 0.7, 0.8, 0.9, 1, 2, 3, 4, 5, 6, 7, 8, 9, 10, 20, 30, 40, 50, 60, 70, 80, 90, 100, 500, 1000, 2000, 3000, 4000, 5000, 6000, 7000, 8000, 9000, or 10000 kDa including all values lying within this range).

In various embodiments, the water-in-oil primary emulsion of step (a) is obtained by homogenization of the aqueous solution with the oil phase including a polymer. In various embodiments, homogenization is performed for 5, 10, 15, 20, 25, 30, 30, 40, 45, 60, 90, 120, 150, 180, 210, 240, 270, 300, 330, 360, 390, 420, 450, 480, 510, 540, 570, 600, 700, 800, 900, or 1000 seconds. In various embodiments, the oil-in-water secondary emulsion of step (b) is obtained by homogenization of the primary emulsion with a solution including one or more surfactants and/or stabilizer. In various embodiments, homogenization is performed for 5, 10, 15, 20, 25, 30, 30, 40, 45, 60, 90, 120, 150, 180, 210, 240, 270, 300, 330, 360, 390, 420, 450, 480, 510, 540, 570, 600, 700, 800, 900, or 1000 seconds. In various embodiments, the water-in-oil primary emulsion of step (a) is obtained by sonication of the aqueous solution with the oil phase including a polymer. In various embodiments, sonication is performed for 5, 10, 15, 20, 25, 30, 30, 40, 45, 60, 90, 120, 150, 180, 210, 240, 270, 300, 330, 360, 390, 420, 450, 480, 510, 540, 570, or 600 seconds.

In various embodiments, the solution including one or more surfactants and/or stabilizers that form an oil-in-water secondary emulsion in step (b) has a pH less than 4.0 or equal to 4.0. In various embodiments, the oil-in-water secondary emulsion has a pH of about pH 1 to about pH 4, about pH 2 to about pH 4, about pH 3 to about pH 4, or about pH 1, about pH 1.5, about pH 2, about pH 2.5, about pH 3, about pH 3.5, or about pH 4 including including all values and ranges therein.

In various embodiments, the oil-in-water secondary emulsion of step (b) is obtained by sonication of the primary emulsion with a solution including one or more surfactants and/or stabilizers. In various embodiments, sonication is performed for 5, 10, 15, 20, 25, 30, 30, 40, 45, 60, 90, 120, 150, 180, 210, 240, 270, 300, 330, 360, 390, 420, 450, 480, 510, 540, 570, or 600 seconds.

In various embodiments, the secondary emulsion is hardened by evaporation. In various embodiments, the evaporation is active evaporation. In various embodiments, the evaporation is passive evaporation. In various embodiments, the active evaporation is vacuum-driven evaporation. In various embodiments, evaporation is performed for 0.25, 0.5, 1, 2, 3, 4, 5, 6, 7, 8, 9, 10, 11, 12, 13, 14, 15, 16, 17, 18, 19, 20, 21, 22, 23, 24, 48, 72, or 96 hours. In various embodiments, the secondary emulsion is hardened by evaporation. In various embodiments, the evaporation is active evaporation. In various embodiments, the evaporation is passive evaporation. In various embodiments, the active evaporation is performed using stirring or under vacuum. In various embodiments, the active evaporation is performed under high-pressure vacuum. In various embodiments, the active evaporation is performed under low pressure vacuum. In various embodiments, evaporation is performed for 0.25, 0.5, 1, 2, 3, 4, 5, 6, 7, 8, 9, 10, 11, 12, 13, 14, 15, 16, 17, 18, 19, 20, 21, 22, 23, 24, 48, 72, or 96 hours. In various embodiments, the evaporation is performed at a pressure of kDa 0.01 to 1000 mBar (e.g., between 0.01, 0.02, 0.03, 0.04, 0.05, 0.1, 0.2, 0.3, 0.4, 0.5, 0.6, 0.7, 0.8, 0.9, 1, 2, 3, 4, 5, 6, 7, 8, 9, 10, 20, 30, 40, 50, 60, 70, 80, 90, 100, 150, 200, 250, 300, 350, 400, 450, 500, 550, 600, 650, 700, 750, 800, 850, 900, 950, or 1000 mBar including all including all values lying within this range. In various embodiments, the evaporation step is designed to remove process solvents such that the level of residual process solvents in the final nanoparticle formulation is about 0.0005% and 100% (e.g., between 0.0005, 0.001, 0.005, 0.01, 0.05, 0.1, 0.2, 0.3, 0.4, 0.5, 0.6, 0.7, 0.8, 0.9, 1, 2, 3, 4, 5, 6, 7, 8, 9, 10, 20, 30, 40, 50, 60, 70, 80, 90, or 100% including all values lying within this range) by weight or volume. In various embodiments, the evaporation step is designed to remove process solvents such that the level of residual process solvents in the final nanoparticle formulation is between 0.0005 and 500,000 ppm (e.g., between 0.0005, 0.001, 0.005, 0.01, 0.05, 0.1, 0.2, 0.3, 0.4, 0.5, 0.6, 0.7, 0.8, 0.9, 1, 2, 3, 4, 5, 6, 7, 8, 9, 10, 20, 30, 40, 50, 60, 70, 80, 90, 100, 1000, 10000, 100000, 200000, 400000, or 500000 ppm including all values lying within this range). In various embodiments, the evaporation step is designed to remove process solvents such that the level of residual process solvents in the final nanoparticle formulation is between 0.0005 and 500,000 ppb (e.g., between 0.0005, 0.001, 0.005, 0.01, 0.05, 0.1, 0.2, 0.3, 0.4, 0.5, 0.6, 0.7, 0.8, 0.9, 1, 2, 3, 4, 5, 6, 7, 8, 9, 10, 20, 30, 40, 50, 60, 70, 80, 90, 100, 1000, 10000, 100000, 200000, 400000, or 500000 ppb including all values lying within this range).

In various embodiments, the filtration, washing, and concentration of particles in step (d) is performed by gel filtration, membrane filtration, dialysis, centrifugation, chromatography, density gradient centrifugation, or combinations thereof. In various embodiments, the step is designed to remove process solvent(s), surfactant(s), and/or stabilizer(s) such that level of residual process solvent(s), surfactant(s), and/or stabilizer(s) in the final nanoparticle formulation is between 0.0005 and 100% (e.g., between 0.0005, 0.001, 0.005, 0.01, 0.05, 0.1, 0.2, 0.3, 0.4, 0.5, 0.6, 0.7, 0.8, 0.9, 1, 2, 3, 4, 5, 6, 7, 8, 9, 10, 20, 30, 40, 50, 60, 70, 80, 90, or 100% including all values lying within this range) by weight or volume. In various embodiments, the step is designed to remove process solvent(s), surfactant(s), and/or stabilizer(s) such that level of residual process solvent(s), surfactant(s), and/or stabilizer(s) in the final nanoparticle formulation is between 0.0005 and 500,000 ppm (e.g., between 0.0005, 0.001, 0.005, 0.01, 0.05, 0.1, 0.2, 0.3, 0.4, 0.5, 0.6, 0.7, 0.8, 0.9, 1, 2, 3, 4, 5, 6, 7, 8, 9, 10, 20, 30, 40, 50, 60, 70, 80, 90, 100, 1000, 10000, 100000, 200000, 400000, or 500000 ppm including all values lying within this range). In various embodiments, the step is designed to remove process solvent(s), surfactant(s), and/or stabilizer(s) such that level of residual process solvent(s), surfactant(s), and/or stabilizer(s) in the final nanoparticle formulation is between 0.0005 and 500,000 ppb (e.g., between 0.0005, 0.001, 0.005, 0.01, 0.05, 0.1, 0.2, 0.3, 0.4, 0.5, 0.6, 0.7, 0.8, 0.9, 1, 2, 3, 4, 5, 6, 7, 8, 9, 10, 20, 30, 40, 50, 60, 70, 80, 90, 100, 1000, 10000, 100000, 200000, 400000, or 500000 ppb including all values lying within this range).

In various embodiments, excipients are added to the nanoparticle composition prior to freeze drying in step (e). In various embodiments, the excipients are buffering agents and/or cryoprotectants. In various embodiments, the excipients are selected from the group consisting of sucrose, mannitol, trehalose, sorbitol, dextran, Ficoll, Dextran 70k, sodium citrate, lactose, L-arginine, or glycine. In various embodiments, the amounts of excipients added to the nanoparticle composition prior to freeze drying is between 0.05 and 100% (e.g., between 0.05, 0.1, 0.2, 0.3, 0.4, 0.5, 0.6, 0.7, 0.8, 0.9, 1, 2, 3, 4, 5, 6, 7, 8, 9, 10, 20, 30, 40, 50, 60, 70, 80, 90, or 100% including all values lying within this range) by weight or volume. As used herein, when an excipient is present at 100% weight or volume, said excipient is present in an equal amount compared to the negatively charged particles disclosed herein. In various embodiments, the amounts of excipients added to the nanoparticle composition prior to freeze drying is between 0.01 and 500 g (e.g., between 0.01, 0.02, 0.03, 0.04, 0.05, 0.1, 0.2, 0.3, 0.4, 0.5, 0.6, 0.7, 0.8, 0.9, 1, 2, 3, 4, 5, 6, 7, 8, 9, 10, 20, 30, 40, 50, 60, 70, 80, 90, 100, 150, 200, 250, 300, 350, 400, 450, or 500 g) per gram of nanoparticles. In various embodiments, the excipients used in the process for manufacturing negatively charged particles are selected to achieve a pH of about 1.0 and 14.0 for the final formulation (e.g., between 1.0, 2.0, 3.0, 4.0, 5.0, 6.0, 7.0, 8.0, 9.0, 10.0, 11.0, 12.0, 13.0, or 14.0) including all values and ranges therein. In various embodiments, the excipients used in the process of manufacturing negatively charged particles yield a pH of about 1.0 to 7.0, or about 2.0 to 7.0, or about 3.0 to 7.0, or about 4.0 to 7.0, or about 5.0 to 7.0, or about 6.0 to 7.0, or between 2.0 to 6.0, or about 3.0 to 6.0, or about 4.0 to 6.0, or about 1.0 to 5.0, or about 2.0 to 5.0, or about 3.0 to 5.0, or about 4.0 to 5.0, or about 1.0 to 4.0, or about 2.0 to 2.0, or about 3.0 to 4.0 for the final formulation, including all values and ranges therein.

In various embodiments, the freeze drying step is performed for about 1 hour to about 2000 hours (e.g., about 1, 2, 3, 4, 5, 6, 7, 8, 9, 10, 15, 20, 25, 30, 35, 40, 45, 50, 75, 100, 150, 200, 300, 400, 500, 600, 700, 800, 900, 1000, 1500, or 2000 hours) including all ranges and values therein. In various embodiments, the freeze drying step used in the process of manufacturing negatively charged particles is designed to yield a moisture level of between 0.0005 and 25% (e.g., between 0.0005, 0.001, 0.005, 0.01, 0.05, 0.1, 0.2, 0.3, 0.4, 0.5, 0.6, 0.7, 0.8, 0.9, 1, 2, 3, 4, 5, 6, 7, 8, 9, 10, 15, 20, or 25%) by weight or volume in the final formulation, including all values and ranges therein.

In various embodiments, the process for manufacturing negatively charged particles yields between about 10 and 10⁹⁹ particles (e.g., 10, 10¹, 10², 10³, 10⁴, 10⁵, 10⁶, 10⁷, 10⁸, 10⁹, 10¹⁰, 10²⁰, 10³⁰, 10⁴⁰, 10⁵⁰, 10⁶⁰, 10⁷⁰, 10⁸⁰, 10⁹⁰, or 10⁹⁹ per vial) including all values and ranges therein.

In various embodiments, the manufacturing batch sizes of negatively charged particles can be scaled up or down. In various embodiments, the manufacturing batch size is between 0.01 g to 100 kg. In various embodiments, the batch size is 0.01 g, 0.1 g, 10 g, 20 g, 40 g, 60 g, 80 g, 100 g, 160 g, 240 g, 320 g, 400 g, 480 g, 560 g, 640 g, 720 g, 800 g, 1000 g, 5 kg, 10 kg, 50 kg or 100 kg including all values and ranges that lie between these values.

“Batch size” as used herein relates to the scale of manufacture depending on the weight of the particles in the final product. The manufacturing process can be altered, scaled up or scaled down. The manufacturing process can be altered, scaled up or scaled down by altering the amount or volume of the solvent, antigens/proteins, polymer, surfactants, stabilizers, cryoprotectants or excipients. The manufacturing process can be scaled up or down by altering the time of homogenization, sonication, evaporation, filtration, concentration, washing or lyophilization.

In certain embodiments, the particles encapsulating antigens are manufactured by nanoprecipitation, co-precipitation, inert gas condensation, sputtering, microemulsion, sol-gel method, layer-by-layer technique or ionic gelation method. Several methods for manufacturing nanoparticles have been described in the literature and are incorporated herein by reference.

Additional aspects and details of the disclosure will be apparent from the following examples, which are intended to be illustrative rather than limiting.

### EXAMPLES

### Example 1: Phase 1b/2a Clinical Trial of Negatively Charged Particles of the Disclosure in Adult Subjects with Metastatic or Advanced Solid Tumors

The present example describes a Phase 1b/2a open-label clinical study to evaluate the safety, tolerability, pharmacodynamics, and efficacy of negatively charged particles (referred to as ONP-302) of the disclosure in adult subjects with treatment refractory metastatic or advanced solid tumors.

The negatively charged particles comprise of PLGA nanoparticles free from other drugs. The negatively charged particles have an average diameter of between 350-800 nm and a negative zeta potential of between −30 mV to −60 mV. The negatively charged particles are supplied as a lyophilized formulation. The negatively charged particles are reconstituted in sterile water for injection and diluted in sterile saline (0.9% sodium chloride, USP) prior to administration. Adult subjects ages ≥18 years are screened prior to enrollment. Subjects meeting all inclusion criteria and no exclusion criteria will be enrolled in the Cohort open at the time to receive ONP-302. The study consists of two parts: Part I (dose escalation) and Part II (dose expansion). Part I will follow a classic 3+3 design to support escalation decisions. 3 dose levels have been defined for escalation:
Dose Level 1: 2 mg/kg ONP-302
Dose Level 2: 4 mg/kg ONP-302
Dose Level 3: 8 mg/kg ONP-302 (up to 650 mg maximum)

In Part I each subject receives the negatively charged particles as a cycle consisting of four doses administered once weekly. The negatively charged particles are administered via intravenous infusion lasting approximately 3-4 hours unless safety concerns require premature discontinuation of infusion in a subject. Subjects are observed for AEs and SAEs, including infusion reactions (IRs), for up to two hours following infusion on each dosing day. The first subject of the first cohort at each dose level will be followed through 2 doses (study Day 8) before additional subjects are enrolled into that cohort. Enrollment of successive subjects in a cohort proceed after the previous subject has received two doses of negatively charged particles with no AEs ≥Grade 2 not resolving to baseline within 48 hours, no related SAEs, and/or no DLTs have been reported following the second dose. Initial cohorts at each dose level consist of 3 patients. When initial enrollment of a cohort is complete, further enrollment will be determined as per the DMC's decision: to escalate to the next dose level; to add an additional set of 3 patients at the current dose level (3+3); or that escalation has concluded. Part I concludes either with the identification of MTD or completion of the planned dose levels without reaching MTD. MTD is defined as the highest dose at which no more than 2/6 patients experience DLT during the initial cycle of treatment.

Once Part I has concluded, one or more dose level at or below the MTD are selected for expanded enrollment in Part II. Decisions regarding which dose level to enroll in Part II may include signs of pharmacodynamic and/or antitumor response from Part I in addition to safety data. Subjects may be added to the expanded dose level(s) (up to a total of 12 subjects per dose level), to further assess safety, tolerability, and pharmacodynamic effects, and to preliminarily assess anti-tumor activity. Approximately 12 subjects are expected to be included in Part I, and up to 18 additional subjects may be included in Part II, giving a total of up to 30 subjects. Subjects tolerating negatively charged particles without signs of progression may be eligible to receive additional cycles of treatment up to a maximum of 6 cycles total at their same dose level, at the discretion of the Investigator.

### Safety Assessments Will Include:

Physical exam at Screening, then symptom-driven physical exam on each dosing day of the first cycle, then on the fourth dosing day of each additional cycle.
Vital signs at Screening, then on each dosing day of the first cycle, then on the fourth dosing day of each additional cycle.
Single 12-lead ECG at Screening, then on each dosing day of the first cycle only.
AEs and SAEs during the entire course of the subject's participation in the study.
Safety labs (chemistry, hematology, coagulation, urinalysis) at Screening, on each dosing day of the first cycle, and then on the fourth dosing day of each additional cycle.
Complement (C3a, C5a, sC5b9, and C1q) and cytokines/chemokines (IL-1b, IL-2, IL-6, IL-7, IL-10, IP 10, MCP-1, and GM-CSF) in case of IRs (collected samples will be analyzed in the event of infusion reaction or other related infusion reaction adverse events with putative complement and cytokine/chemokine involvement). Samples are collected pre-dose on Day 1 to establish Baseline. Samples are collected at approximately 15 minutes post onset, 2 hours (+/−5 mins) post onset and approximately 24 (+/−2 hours) hours after the infusion reaction or related adverse event. Patients will stay in-unit for up to 8 hours post any AE related to infusion to monitor recovery.

### Pharmacodynamic (PD) Assessments Will Include:

Frequency of T cells and NK cells (total T cells/total leukocytes and total NK cells/total leukocytes) in blood pre-dose (Baseline) and 24-hours post-dose on each dosing day of the first cycle, then pre-dose Day 22 and 24-hours post-dose on the fourth dosing day of each additional cycle.
Frequency of activated T cells and NK cells (activated T cells/total T cells and activated NK cells/total NK cells) in blood pre-dose (Baseline) and 24-hours post-dose on each dosing day of the first cycle, then pre-dose Day 22 and 24-hours post-dose on the fourth dosing day of each additional cycle.
Number of CTCs (per mL blood) at Screening (Baseline), then 24 hours after every fourth dose of each cycle.
IL-15 gene expression levels in APCs in blood pre-dose (Baseline) and 24-hours post-dose on each dosing day of the first cycle, then pre-dose Day 22 and 24-hours post-dose on the fourth dosing day of each additional cycle.
Levels of NETs in blood pre-dose (Baseline) and 24-hours post-dose on each dosing day of the first cycle, then pre-dose Day 22 and 24-hours post-dose on the fourth dosing day of each additional cycle.
Neutrophil-to-lymphocyte ratio (NLR) pre-dose at Screening (Baseline) and then 24 hours after every fourth dose of each cycle.
Serum levels of cytokines and chemokines (soluble IL-15, IL-17, TNF-α, IFN-γ, MIP-1α, MIP-1β, RANTES, TGF-β1, TGF-β2, VEGF, and M-CSF) pre-dose (Baseline) and 24-hours post-dose on each dosing day of the first cycle, then pre-dose Day 22 and 24-hours post-dose on the fourth dosing day of each additional cycle.

### Clinical Assessments Will Include:

Tumor response per RECIST v1.1 8 weeks after initiation of treatment and every 8 weeks while on study.

### Pharmacokinetic (PK) Assessments Will Include:

Plasma concentration of negatively charged particles PD cytokine biomarker IL-8 pre-dose and at 0.5, 2 (+/−5 mins), and 24 hours (+/−2 hours) after IV administration of negatively charged particles on Day 1.

### Criteria for Inclusion:

Males and non-pregnant females, ages ≥18 years.
ECOG performance status 0-2.
Signed written informed consent obtained prior to study procedures.
Relapsed/refractory metastatic or advanced solid tumor for which no standard therapy exists.
Measurable disease per RECIST v1.1 criteria.
Adequate organ function as defined by:
   Aspartate aminotransferase (AST) and alanine aminotransferase (ALT)≤2.5× institution's ULN for patients with no concurrent liver metastases, OR ≤5.0× institution's ULN for patients with concurrent liver metastases.
   Total bilirubin ≤1.5×ULN, except in patients with documented Gilbert's Syndrome who must have a total bilirubin ≤3×ULN.
   Hemoglobin ≥9 g/dL without red blood cell transfusion for at least two weeks.
   LVEF ≥45%.
   ANC ≥10×10³/μL and platelet count ≥100×10³/μL without myeloid growth factor support or transfusion, respectively, for at least one week.
Males and females of child-bearing potential (WOCBP) must be willing to practice a highly effective method of contraception that may include, but is not limited to, abstinence, sex only with persons of the same sex, monogamous relationship with vasectomized partner, vasectomy, hysterectomy, bilateral tubal ligation, licensed hormonal methods, or intrauterine device (IUD).
Willing to comply with all study procedures for the duration of the study.

### Criteria for Exclusion:

Primary CNS malignancy or known CNS involvement.
Prior unrelated malignancy within 3 years of study procedures, except basal cell carcinoma or cancers
in situ.
Prior course of standard therapy or investigational drug within 28 days or 5 half-lives, whichever is shorter, prior to initiation of study treatment.
Concomitant conditions requiring immune suppression therapies.
Known or suspected pregnancy or nursing.
Clinically significant, active, and uncontrolled infection including HIV, Hepatitis B, or Hepatitis C infection.
PT, PTT >1.5×ULN.
Clinically significant coagulopathy or uncontrolled hypertension at investigator discretion.
History of grade ≥3 immune related AE that has not resolved to grade <2.
Known sensitivity to any components of negatively charged particles.
Other known comorbidities that, in the opinion of the Investigator, would pose an unwarranted safety risk.
The following potential comorbid conditions:
   Subjects with a history of cerebrovascular accident.
   Subjects with abnormal QTc (>450 ms in males, >470 ms in females).
   Subjects with known prior myocardial infarction (occurring at any time in the past), as defined by any of the following criteria:
      Development of known new pathological Q waves with or without symptoms.
      Imaging evidence of a region of loss of viable myocardium that is thinned and fails to contract, in the absence of a non-ischemic cause.
      Pathological findings of a healed or healing myocardial infarction.
   Subjects with chronic kidney disease, as defined by Glomerular Filtration Rate (GFR)<60 mL/min/1.73 m² for at least 3 months where GFR is calculated based on the CKD-EPI equation:

GFR=141×min(Scr/κ,1)α×max(Scr/κ,1)−1.209×0.993Age×1.018 [if female]×1.159 [if black] where:
Scr is serum creatinine in mg/dL
κ is 0.7 for females and 0.9 for males
α is −0.329 for females and −0.411 for males
Min indicates the minimum of Scr/κ or 1
Max indicates the maximum of Scr/κ or 1
Subjects who have received administration of any live vaccine (other than intranasal Influenza) within 28 days or subunit vaccine within 14 days prior to Screening or are planning to receive any vaccination at any time during the study. Subjects who have received any COVID-19 vaccine within 14 days prior to Screening. Subjects who have received the first dose of any COVID-19 vaccine may not screen for the study until 14 days following their last dose of the vaccine if applicable.

### Example 2: Determining the Optimal Dose Level and Dosing Frequency of Negatively Charged Particles for Effective Inhibition of Tumor Growth Using the MC38 Mouse Tumor Model

The aim of this study was to identify the dose level and dose administration frequency at which optimal anti-tumor efficacy is observed using the syngeneic MC38 colon adenocarcinoma mouse tumor model. MC38 tumor cells (1.0×10⁶ cells) were implanted subcutaneously into 6-8-week-old C57BL/6 mice. When tumors reached an average volume of 50 mm³, mice were treated with Saline (Control) or different dose levels of negatively charged particles (5 mg/kg, 25 mg/kg, 50 mg/kg, and 125 mg/kg mouse dose: 0.4 mg/kg, 2 mg/kg, 4 mg/kg, or 10 mg/kg HED, respectively). Mice were monitored for tumor growth and survival.

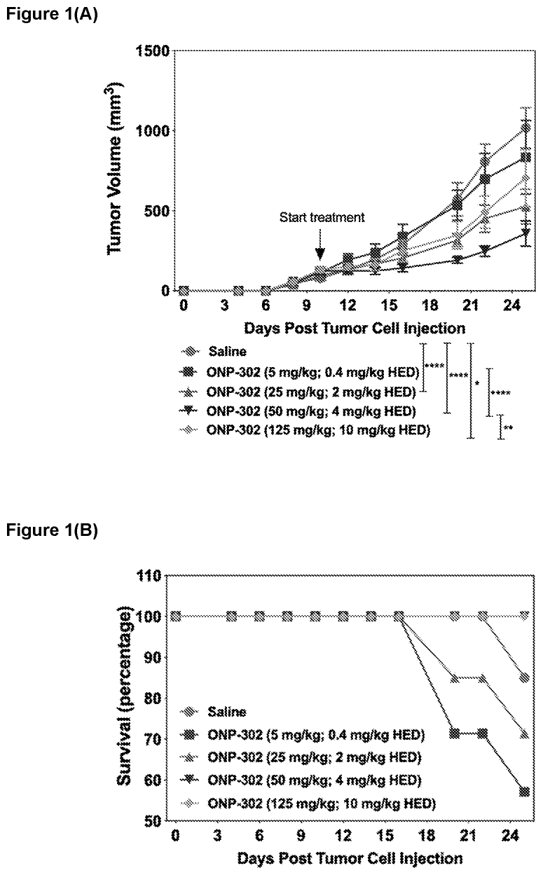

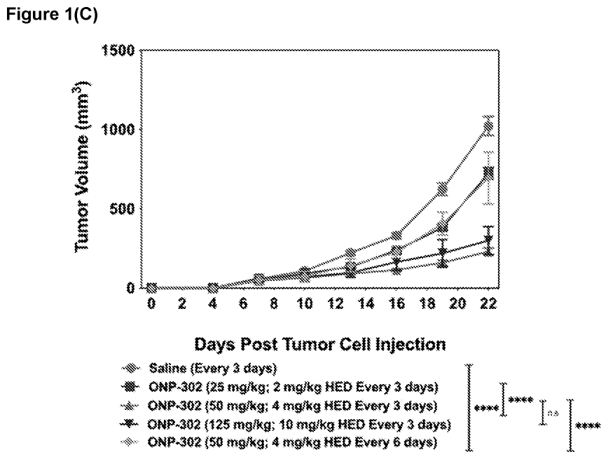

As shown in FIG. 1A, treatment with negatively charged particles (referred to as ONP-302) at 50 mg/kg mouse dose (4 mg/kg HED) led to a statistically significant 2.9× reduction in tumor growth when compared to Control treatment. In comparison, negatively charged particle treatment at lower dose levels tested (5 mg/kg and 25 mg/kg mouse dose; 0.4 mg/kg and 2 mg/kg HED, respectively) did not show the same efficacy at inhibiting tumor growth as that observed at 50 mg/kg mouse dose (4 mg/kg HED). At the highest dose level of 125 mg/kg mouse dose (10 mg/kg HED), negatively charged particles did not show improved efficacy at inhibiting tumor growth when compared to the 50 mg/kg mouse dose (4 mg/kg HED). As shown in FIG. 1B, treatment with negatively charged particles at 50 mg/kg mouse dose (4 mg/kg HED) improved survival compared to Control treatment. In comparison, negatively charged particles treatment at lower dose levels (5 mg/kg and 25 mg/kg mouse dose; 0.4 mg/kg and 2 mg/kg HED, respectively) did not show efficacy at improving survival. At the highest 125 mg/kg mouse dose level (10 mg/kg HED), negatively charged particles survival efficacy was similar to that at 50 mg/kg mouse dose (4 mg/kg HED) level. In a separate experiment, the efficacy of negatively charged particles at 50 mg/kg mouse dose (4 mg/kg HED) administered once every 3 days was compared to the same dose level administered once every 6 days. As shown in FIG. 1C, negatively charged particles administered less frequently (once every 6 days) led to a 1.5× reduction in tumor growth which was suboptimal compared to administration once every 3 days which led to a 4.4× reduction in tumor growth (N=5 per group).

In conclusion, a bell-shaped dose response was observed. Negatively charged particles at 50 mg/kg mouse dose (4 mg/kg HED) administered therapeutically once every 3 days is optimal for inhibiting tumor growth and improving survival in the MC38 mouse tumor model.

### Example 3: Determining the Optimal Dose Level and Dosing Frequency of Negatively Charged Particles for Effective Inhibition of Tumor Growth Using the B16F10 Mouse Tumor Model

The aim of this study was to identify the negatively charged particles dose level and dose administration frequency at which optimal anti-tumor efficacy is observed using the syngeneic B16.F10 (B16) melanoma mouse tumor model. B16 tumor cells (0.5×10⁶ cells) were implanted subcutaneously into 6-8-week-old C57BL/6 mice. When tumor reached an average volume of 50 mm³, mice were treated with Saline (Control) or different dose levels of ONP-302 (25 mg/kg, 50 mg/kg, and 125 mg/kg mouse dose: 2 mg/kg, 4 mg/kg, or 10 mg/kg HED, respectively) once every 3 days.

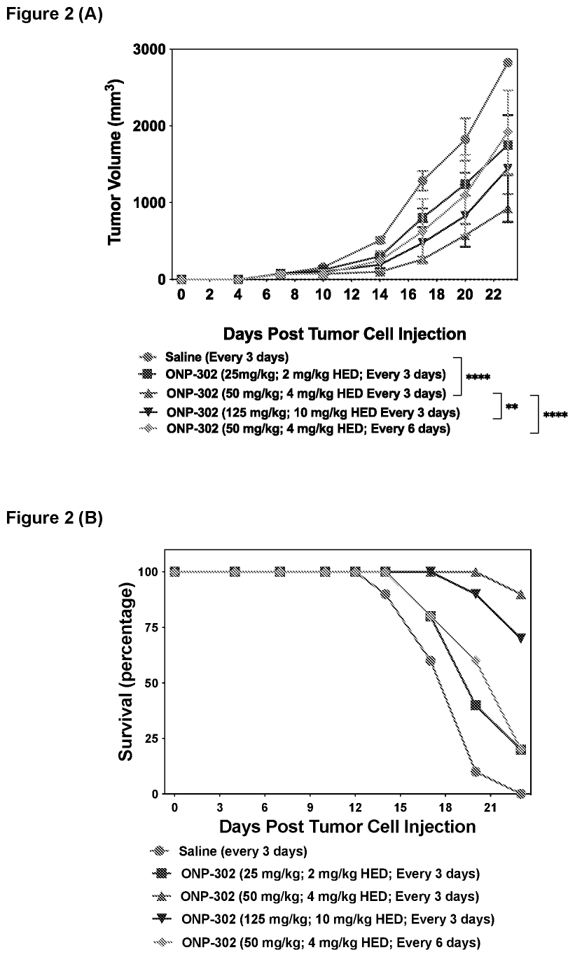

As shown in FIG. 2A, treatment with negatively charged particles (referred to as ONP-302) at 50 mg/kg mouse dose (4 mg/kg HED) administered once every 3 days led to a statistically significant 3× reduction in tumor growth when compared to Control treatment (p<0.05). Increasing negatively charged particles dose level to 125 mg/kg (10 mg/kg HED) did not show improved efficacy at inhibiting tumor growth when compared to 50 mg/kg mouse dose (4 mg/kg HED). As shown in FIG. 2B, treatment with negatively charged particles at 50 mg/kg mouse dose (4 mg/kg HED) administered once every 3 days led to improved survival when compared to Control treatment (p<0.05). In comparison, negatively charged particles treatment at the lower dose level of 25 mg/kg (2 mg/kg HED) did not show efficacy at improving survival. Increasing negatively charged particles dose level to 125 mg/kg mouse dose (10 mg/kg HED) did not show improved survival efficacy when compared to the 50 mg/kg mouse dose (4 mg/kg HED). In the same experiment, the efficacy ONP-302 at 50 mg/kg mouse (4 mg/kg HED) administered once every 3 days was compared to the same dose level administered less frequent administration (once every 6 days). As shown in FIG. 2A, negatively charged particles administration once every 6 days resulted a 1.5× reduction in tumor growth which was suboptimal when compared to administration once every 3 days which led to a 3× reduction in tumor growth. Similarly, as shown in FIG. 2B, negatively charged particles administration once every 6 days demonstrated suboptimal efficacy at improving survival when compared to administration once every 3 days.

In conclusion, a bell-shape dose response was observed. ONP-302 at 50 mg/kg mouse dose (4 mg/kg HED) administered therapeutically once every 3 days is optimal for inhibiting tumor growth and improving survival in the B16 mouse tumor model.

### Example 4: The Anti-Tumor Efficacy of Negatively Charged ONP-302 Nanoparticles is Dependent on a Functional Adaptive Immune System

The aim of this study was to evaluate ONP-302 efficacy at inhibiting primary tumor growth and metastases in the presence and absence of a functional adaptive immune response using the 4T1 orthotopic tumor model. ONP-302 efficacy at inhibiting primary tumor growth and metastasis was evaluated using the 4T1 orthotopic tumor model using Rag1 knockout (Rag1^{-/-}) mice which cannot mount a functional adaptive immune response involving T cells and NK cells.

Approximately, 0.1×10⁶ 4T1 tumor cells expressing luciferase were orthotopically implanted into the mammary fat pads of female 6-8-week-old female WT or Rag 1^{-/-} BALB/c mice. Mice were randomized into treatment groups and administered Saline (Control) or ONP-302 (50 mg/kg mouse dose: 4 mg/kg HED) beginning Day 1 post-tumor implantation. ONP-302 or Saline was administered via intravenous tail vein injection once every 3 days. Tumor growth was monitored by measuring tumor volumes. On Day 21 post-tumor inoculation, mice were euthanized and primary tumor metastasis to the lungs was evaluated by assaying bioluminescence signal from 4T1 tumor cells using the IVIS® imaging system.

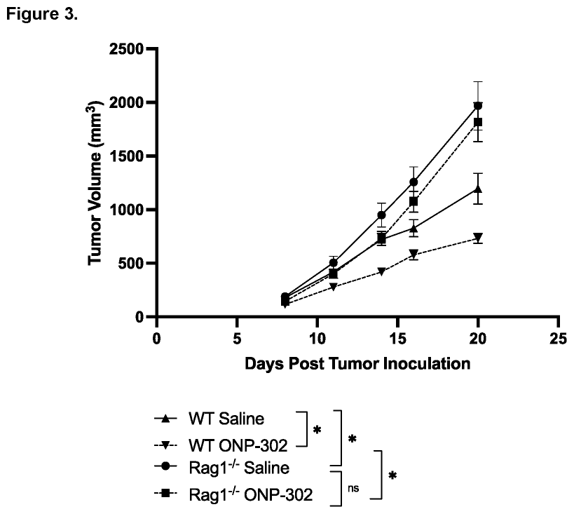

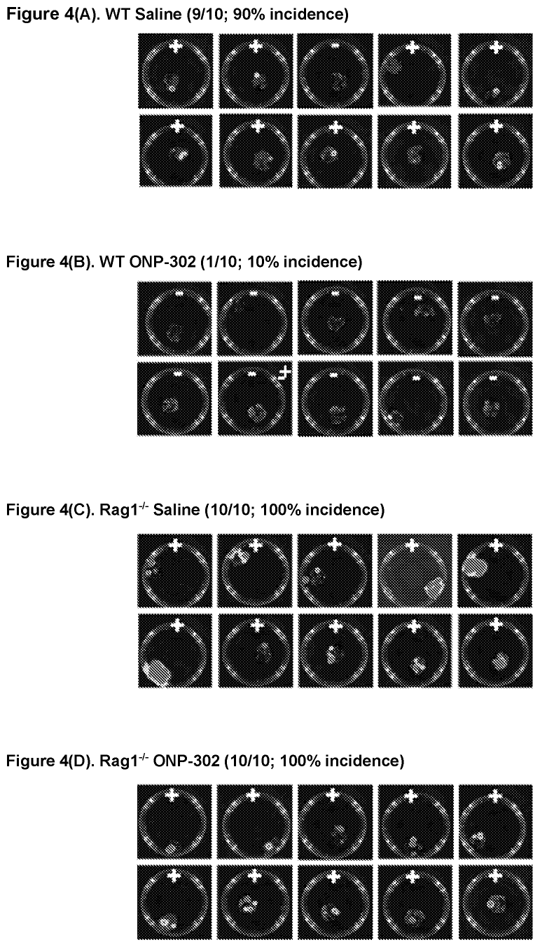

As shown in FIG. 3, treatment with ONP-302 at 50 mg/kg (4 mg/kg HED) in WT tumor bearing mice led to a statistically significant 1.6× reduction in tumor growth when compared to WT tumor bearing mice administered Saline (Control)(p<0.05). In contrast, ONP-302 treatment at the same dose level demonstrated no efficacy in the Rag1^{-/-} tumor bearing mice. As shown in FIG. 4, 9/10 (90%) mice in the Saline (Control) group exhibited lung metastases while only 1/10 (10%) mice in the ONP-302 group exhibited lung metastases. In contrast, ONP-302 demonstrated no efficacy at inhibiting primary tumor metastasis to the lungs in the Rag1^{-/-} tumor bearing mice. As shown in FIGS. 4C and 4D, 10/10 (100%) mice in both Saline (Control) and ONP-302 (50 mg/kg mouse dose; 4 mg/kg HED) treated Rag1^{-/-} tumor bearing mice exhibited evidence of lung metastases. In conclusion, the results of this study demonstrate that ONP-302 (50 mg/kg mouse dose; 4 mg/kg HED) efficacy at inhibiting tumor growth and metastasis in the orthotopic 4T1 breast cancer model is dependent on the presence of a functional adaptive immune system. These results are supportive of ONP-302 anti-tumor mechanism of action which overcomes immune suppression via activation of anti-tumor T cells and NK cells.

### Example 5: Negatively Charged ONP-302 Particles Induce Gene Expression Changes in Immune Cells in the Lungs Associated with Inhibition of Primary Orthotopic 4T1 Tumor Metastasis to the Lungs

To understand the ONP-302 induced immunological changes associated with inhibition of primary orthotopic 4T1 tumor metastasis to the lungs, single-cell RNA sequencing (scRNA-seq) was performed from lung tissues of 4T1 tumor bearing mice administered ONP-302 or saline.

Approximately, 0.1×10⁶ 4T1 tumor cells expressing luciferase were orthotopically implanted into the mammary fat pads of female 6-8-week-old female BALB/c mice. Mice were treated with ONP-302 or Saline once every three days beginning on Day 1 after tumor implantation. Mice were sacrificed on Day 21 post-tumor implantation and lungs were harvested and processed for scRNA-seq.

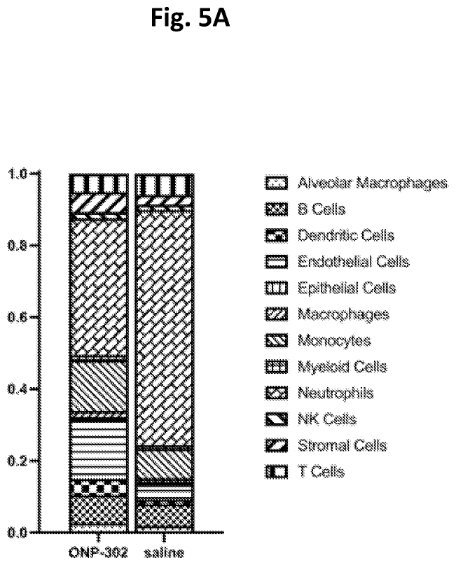

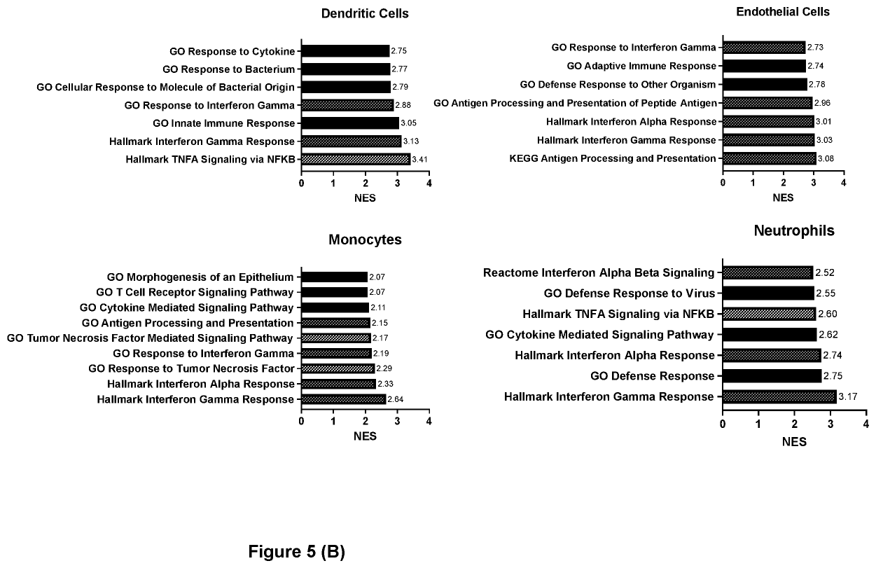

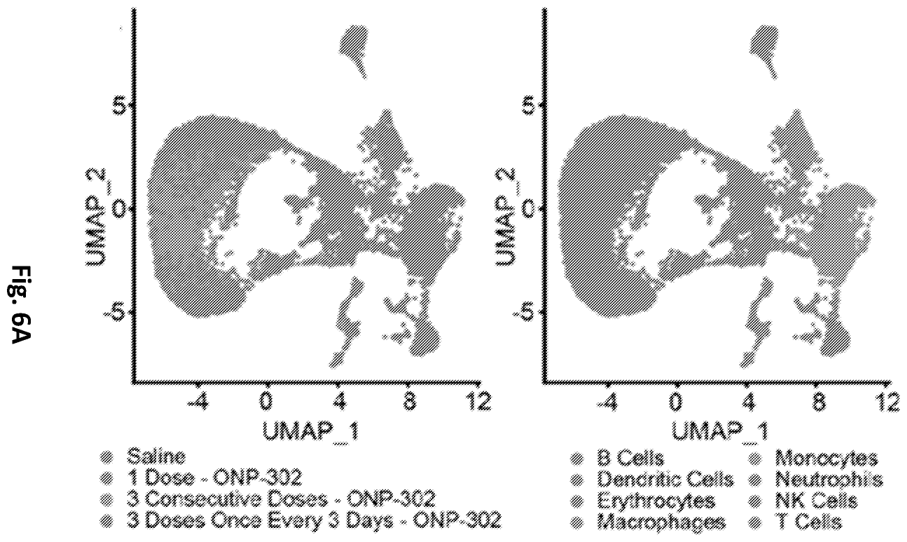

As shown in FIG. 5A, treatment with ONP-302 led to a decrease in the number of neutrophils and an increase in the number of endothelial cells present in the lung tissue. Phenotypic analysis of cell populations within the lungs using Gene Set Enrichment Analysis (GSEA) revealed that ONP-302 treatment induced the greatest phenotypic changes in endothelial cells, dendritic cells, monocytes, and neutrophils. As shown in FIG. 5B, ONP-302 treatment resulted in the induction of interferon-, TNF-, and antigen-associated gene expression signature indicating a shift to an anti-tumor environment in the lung tissue which was associated with inhibition of primary tumor metastasis to the lungs.

In conclusion, these results indicate that the anti-metastasis mechanism of action of ONP-302 is likely via induction of shift from immune suppressive to a more anti-tumor pro-inflammatory environment at metastatic sites.

### Example 6: Negatively Charged ONP-302 Nanoparticles Induce the Activation of cGAS/STING Pathway in Myeloid Cells

cGAS/STING pathway is essential for control of both bacterial and viral pathogens, and cGAS/STING can also be triggered by host genomic DNA in the presence of tumor cells. Consequently, the use of cGAS/STING pathway agonists has been suggested to be a potential therapeutic for the treatment of cancer. Dendritic cell sensing of tumor DNA via cGAS/STING has been shown to be essential for NK cell and CD8⁺ T cell control of tumors.

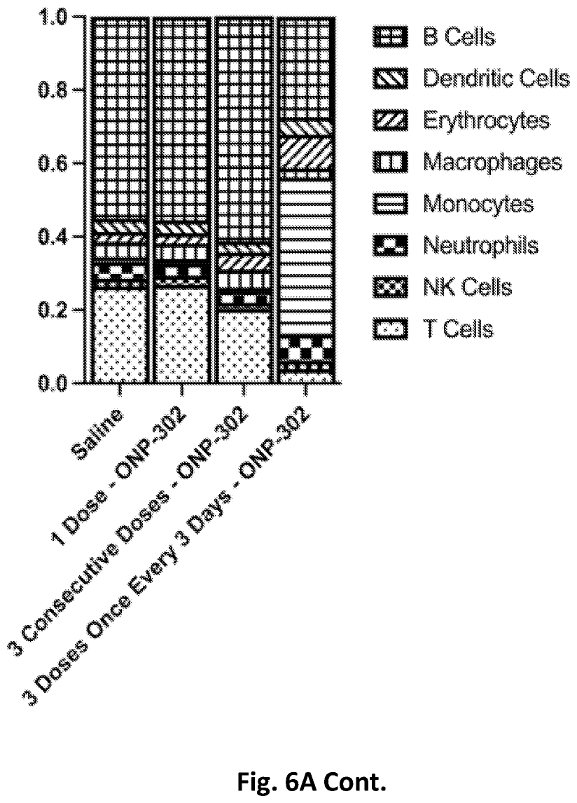

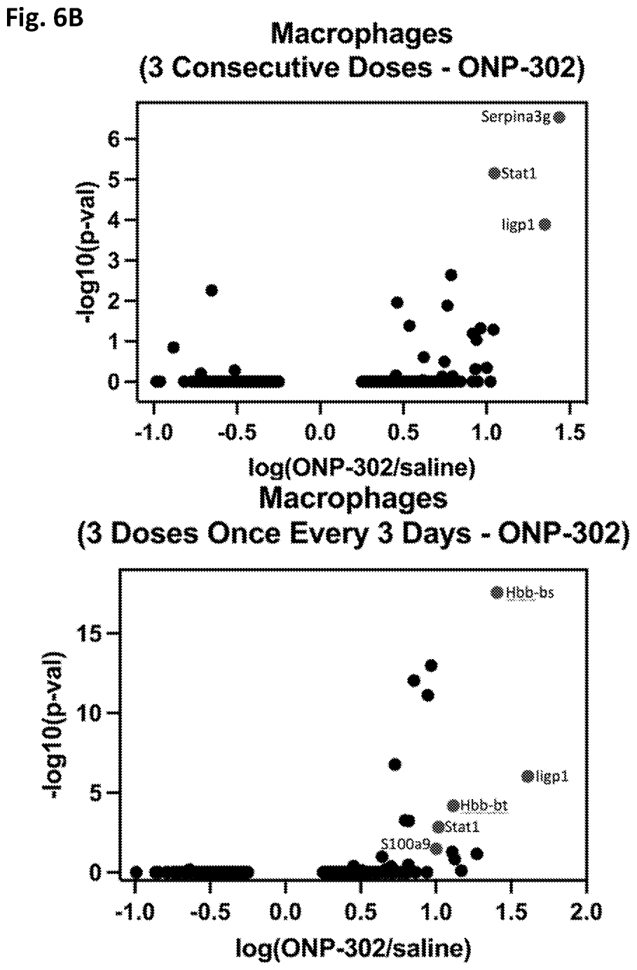

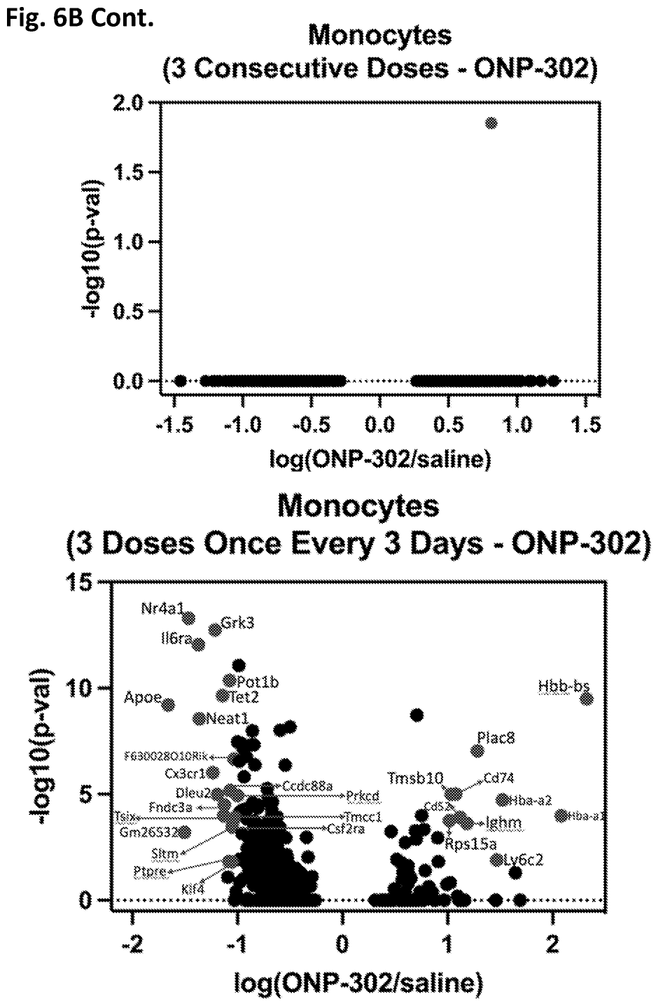

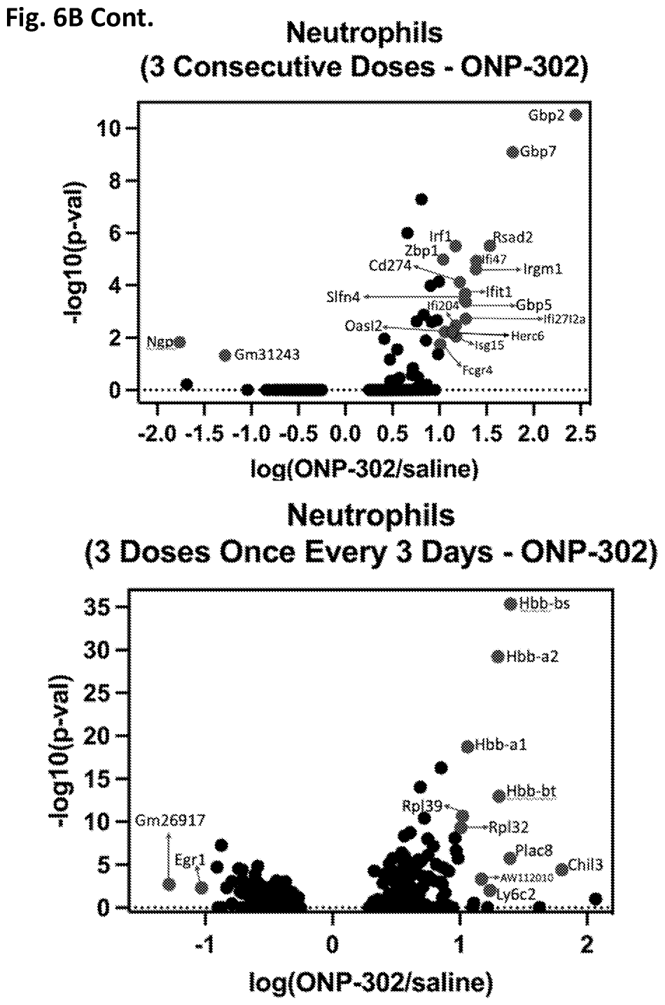

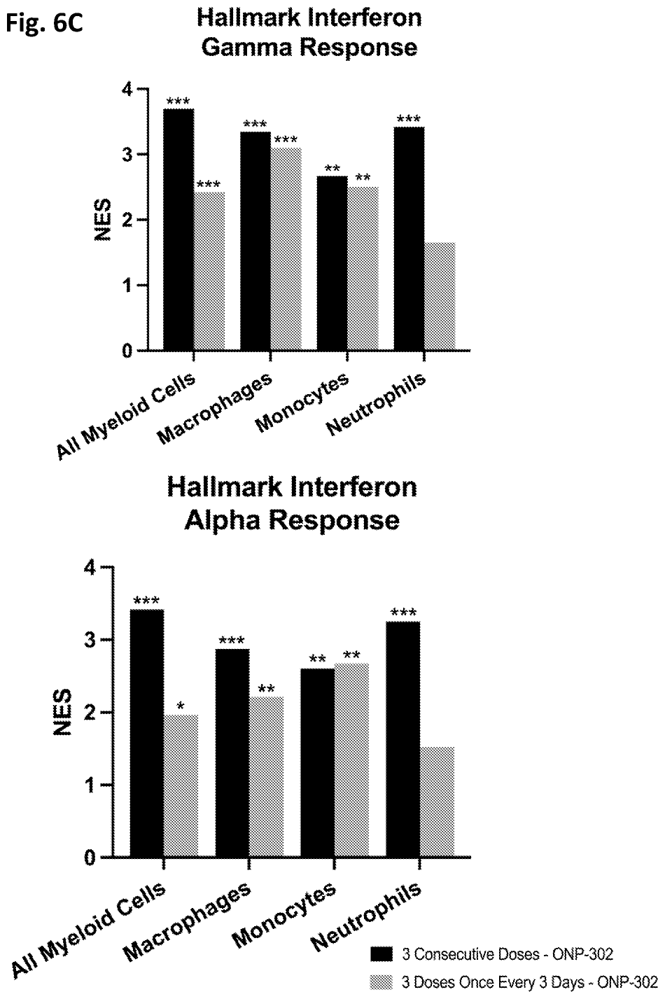

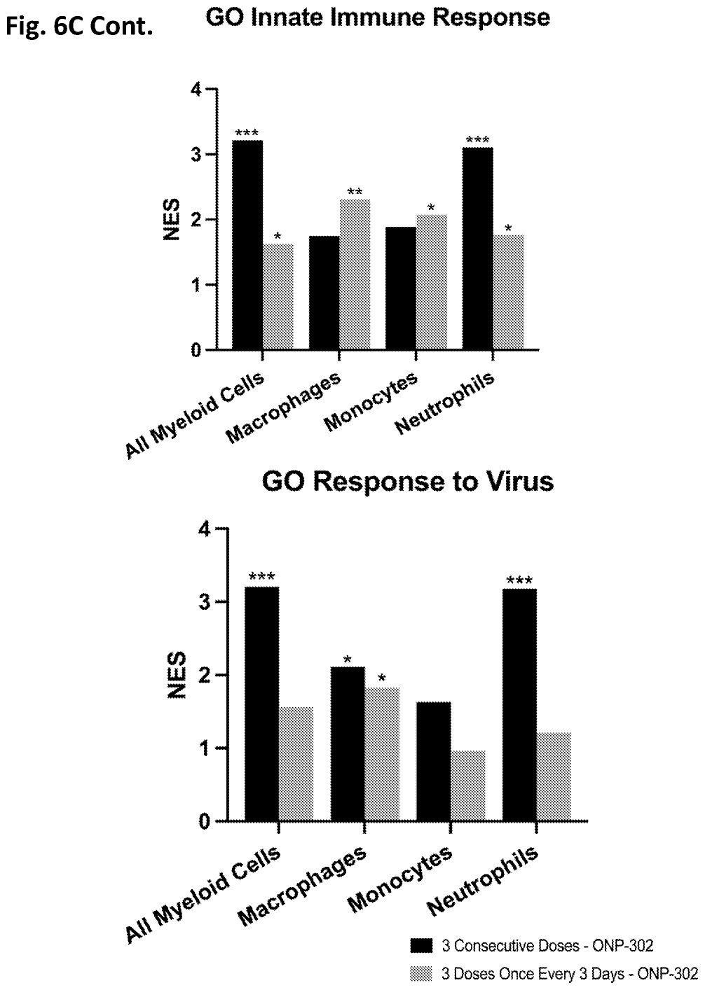

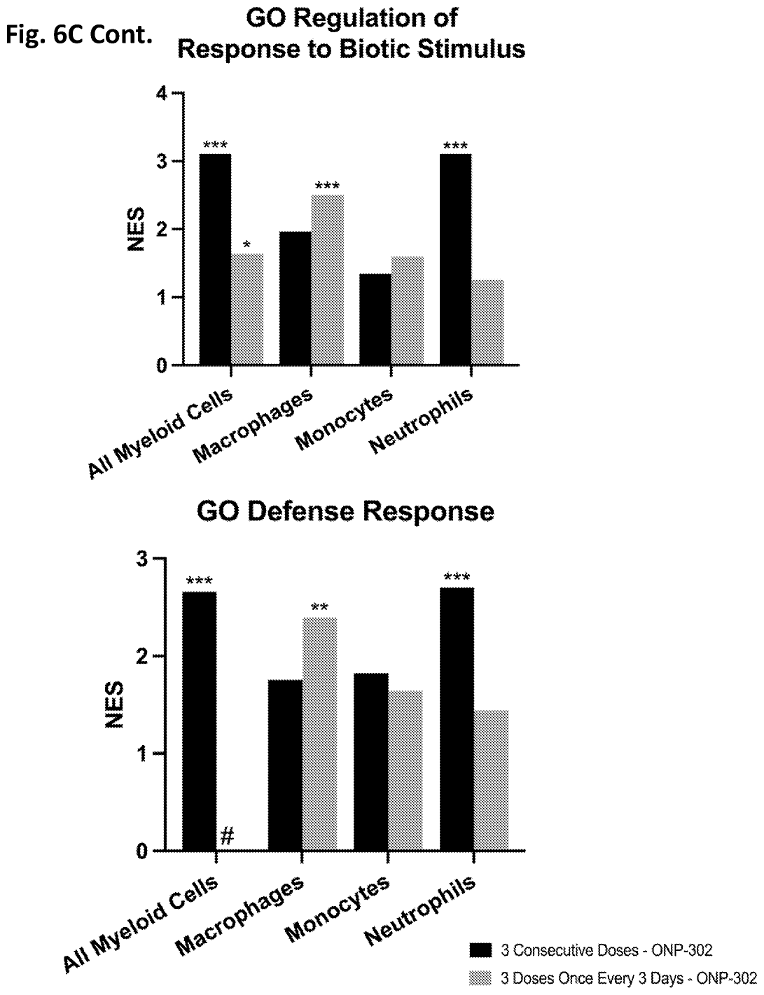

The aim of this study was to evaluate the effect of negatively charged ONP-302 nanoparticles on myeloid cells. To do so, single-cell RNA sequencing (scRNAseq) was performed on splenic leukocytes from naïve C57BL/6 mice injected i.v. with saline, one dose of ONP-302, three doses of ONP-302 on three consecutive days, or three doses of ONP-302 administered every three days. Treatment with three doses of ONP-302 (1 mg/dose) either on consecutive days or every three days induced significant shifts in cell population clustering as compared to saline treatment alone. The treatment of mice on three consecutive days induced an increase in the proportion of B cells, while treating mice every 3 days induced an increase in monocytes, macrophages, and neutrophils (FIG. 6A).

To determine the transcripts and pathways altered by three doses of ONP-302 treatment as compared to saline treatment alone, differential transcript expression was analyzed for macrophages, monocytes, and neutrophils. The present data show that three doses of ONP-302 induced significant alterations in transcripts expressed by macrophages, monocytes, and neutrophils (FIG. 6B). When the differentially expressed transcripts were further analyzed, a significant increase in transcripts associated with IFN-g, IFN-a, the activation of the innate immune response, and cellular pathways associated with anti-viral immune response was identified (FIG. 6C). The present findings suggest that treatment of C57BL/6 mice with three doses of ONP-302 every three days activates cellular pathways within splenic myeloid cells that would be advantageous for immune cell control of tumor growth.

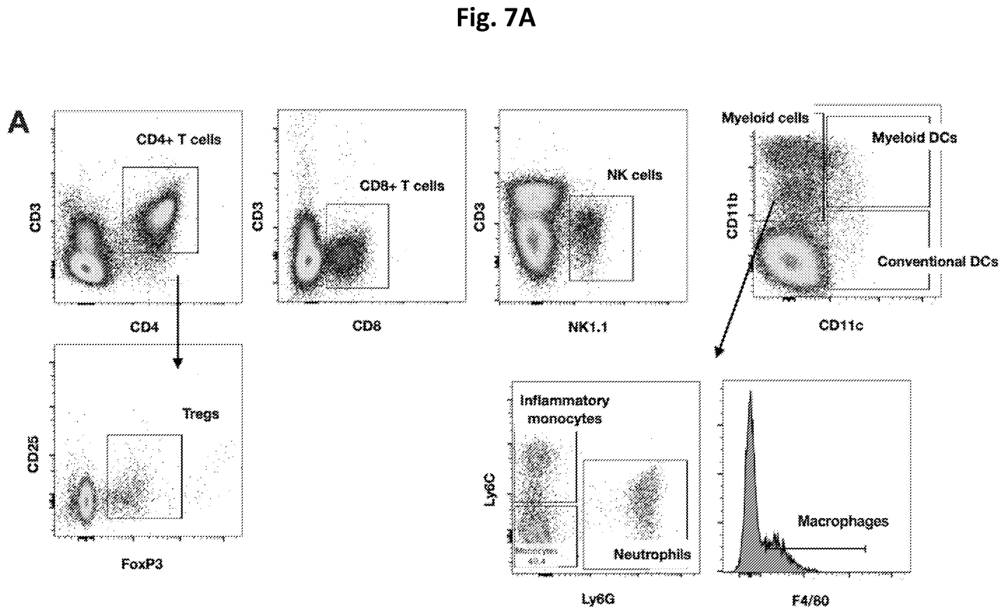

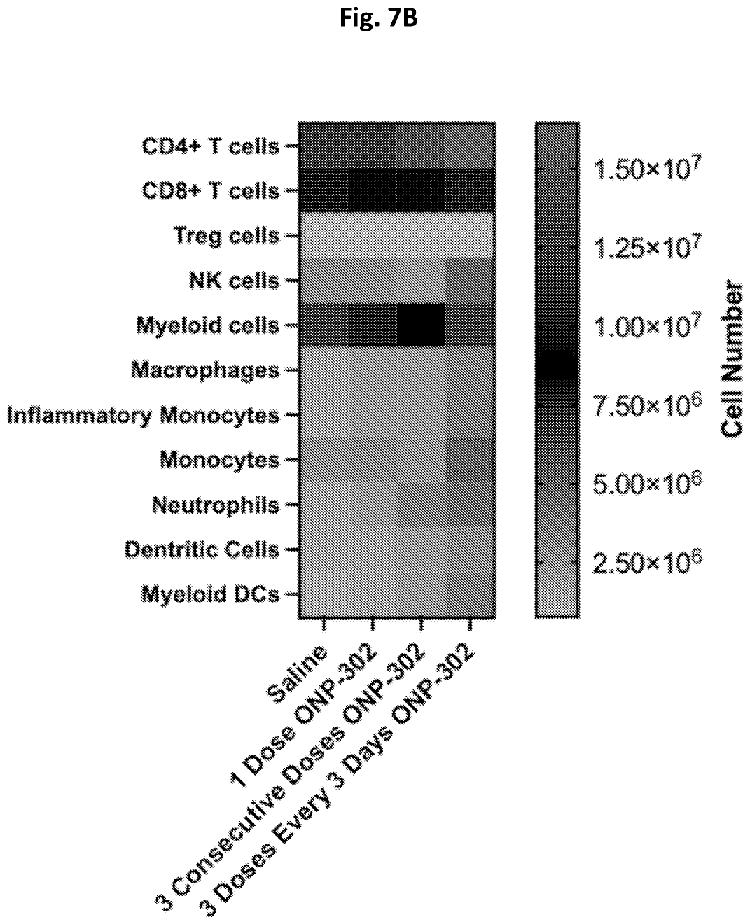

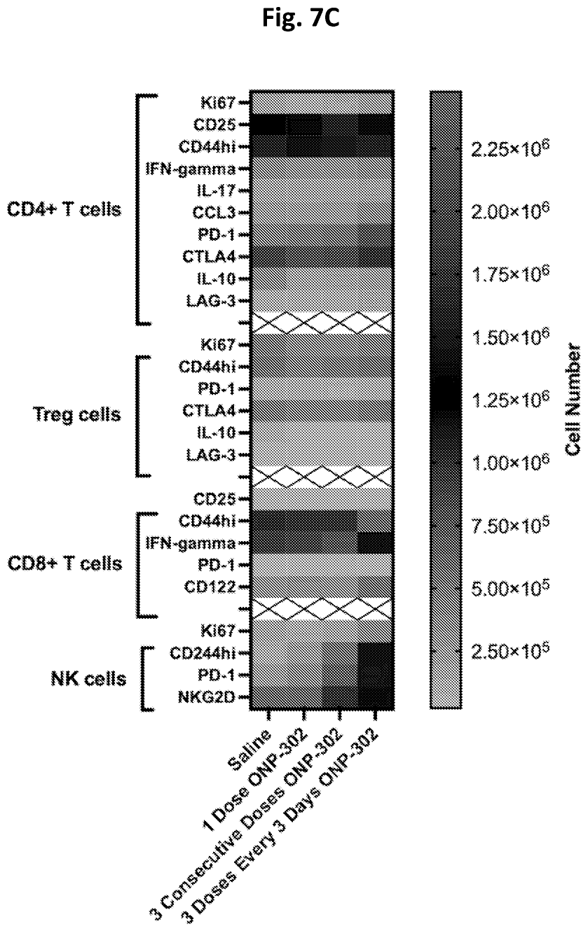

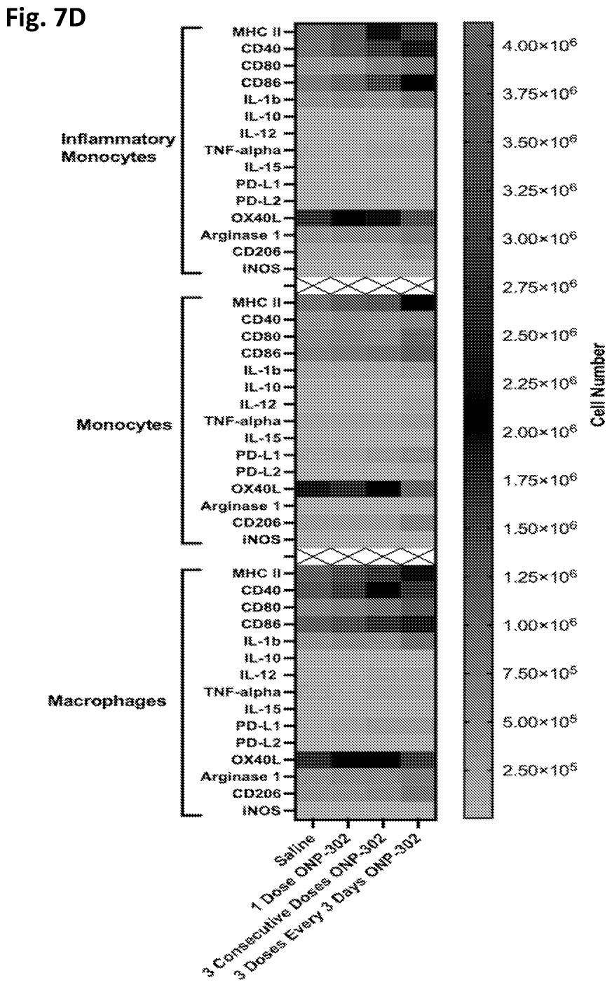

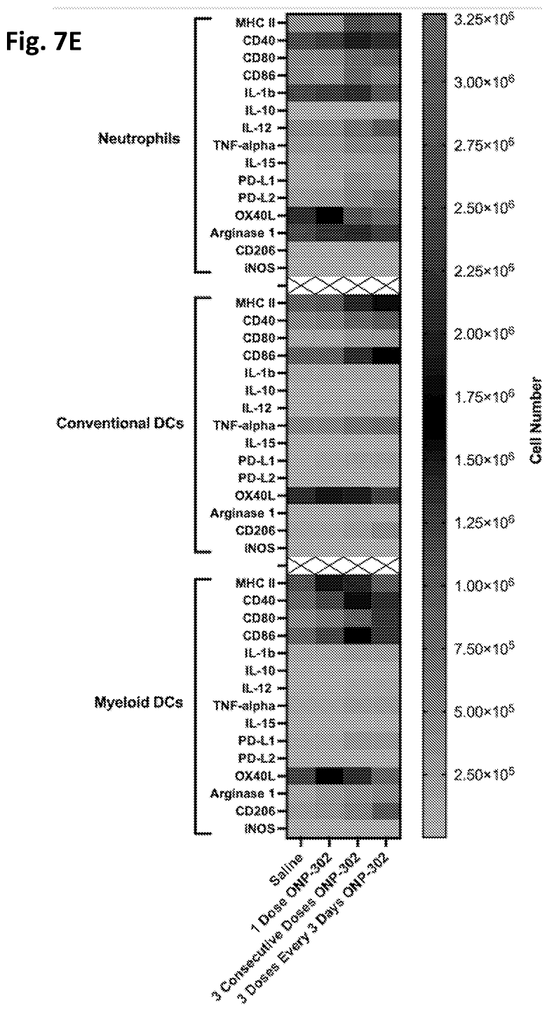

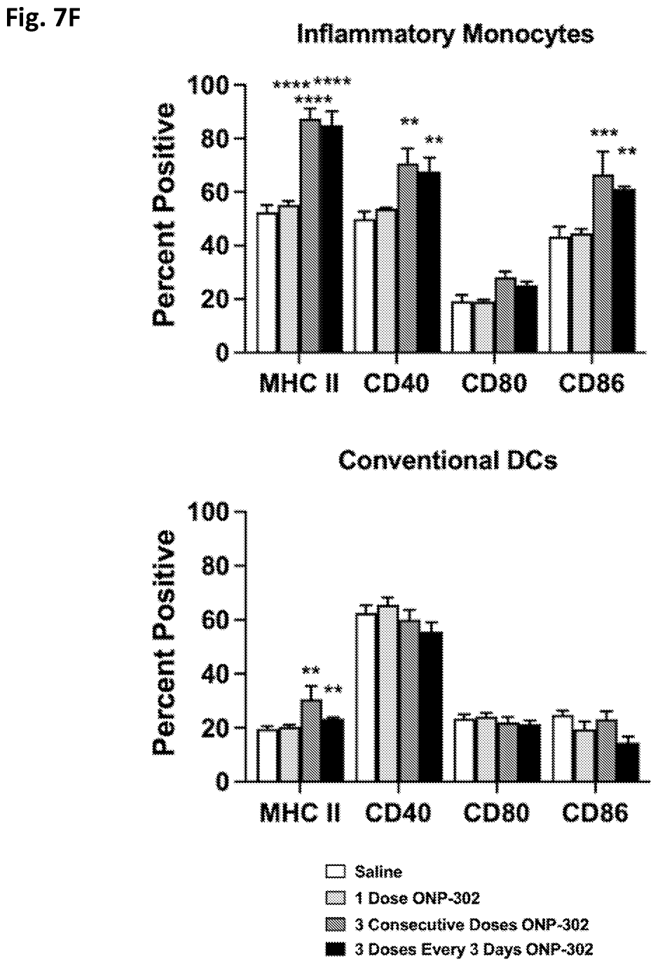

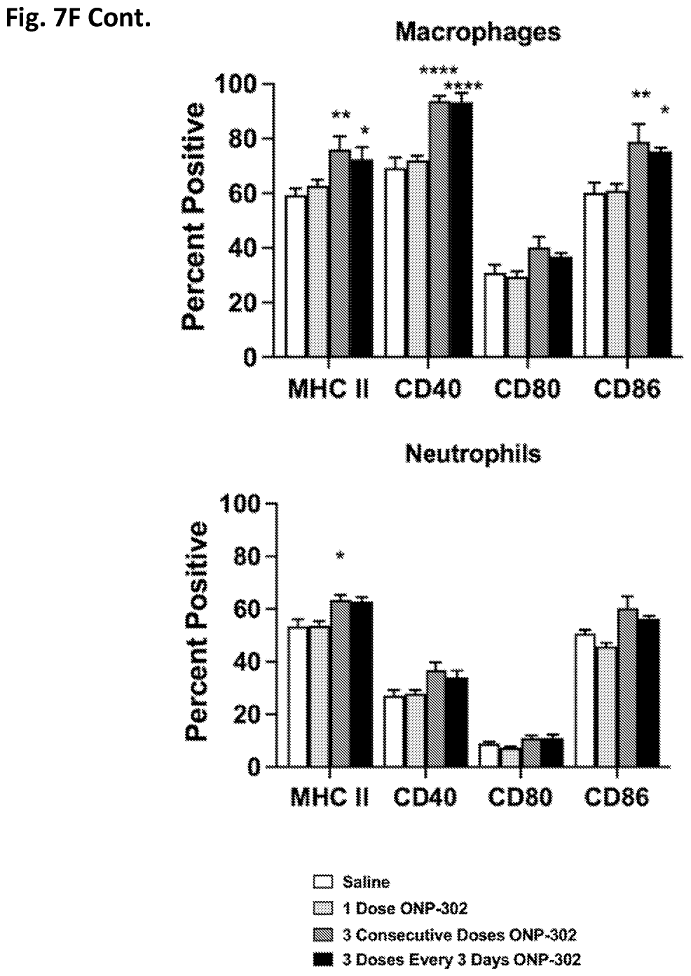

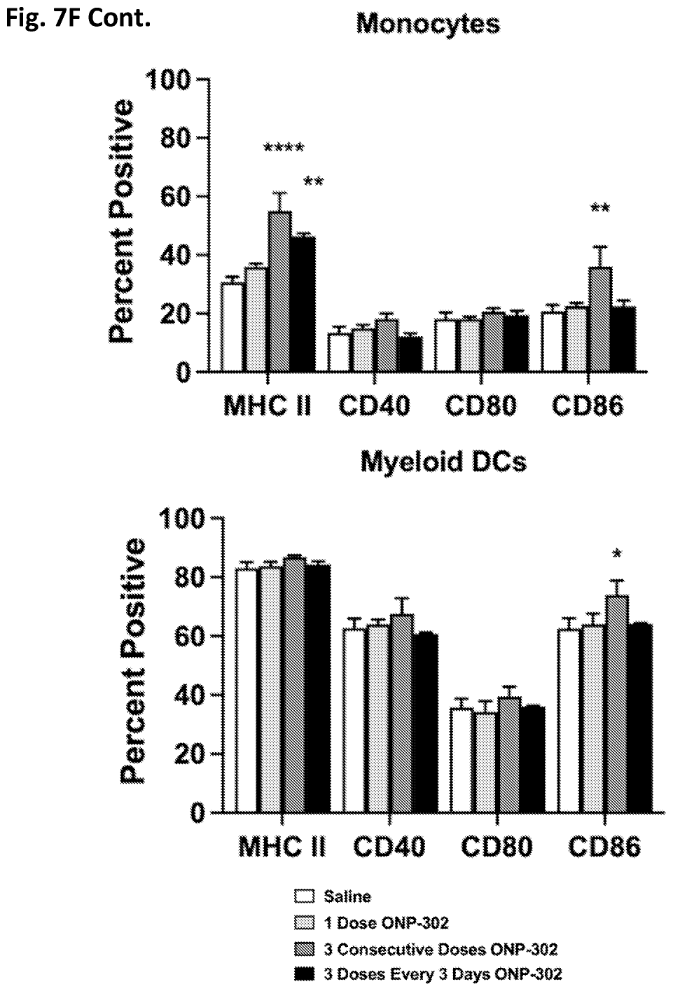

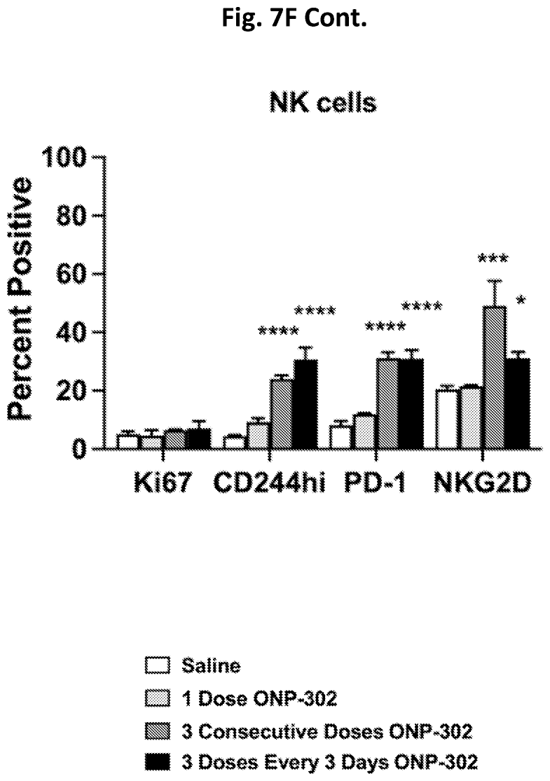

To further confirm and validate the results of the scRNAseq, using the gating schema shown in FIG. 7A, we assessed both the number (FIG. 7B) and phenotype of CD4⁺ T cells, CD8⁺ T cells and NK cells (FIG. 7C), monocytes and macrophages (FIG. 7D), as well as other myeloid populations (FIG. 7E). Of note, repeated dosing of ONP-302 every 72 hours significantly upregulated costimulatory molecules such as CD40 and CD86 on myeloid populations as well as inducing a more activated phenotype on NK cells which significantly upregulated CD244(2B4), PD-1, and NKG2D (FIG. 7F). Taken together, these data indicate that multiple treatments of ONP-302 lead to an increase in pro-inflammatory myeloid cell signatures which lead to downstream increases in NK cell activation and possible function.

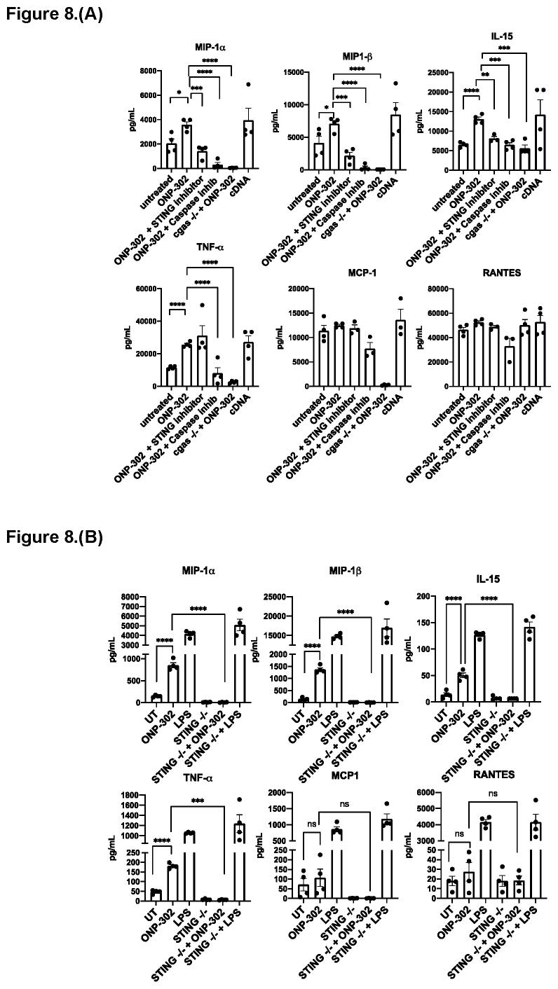

To test whether cGAS/STING signaling was responsible for the transcriptional signatures identified following ONP-302 treatment, RAW cells were cultured in the presence of ONP-302 for 24 hours. Following culture, supernatants were collected to determine the levels of secreted cytokines and chemokines. We observed significant increase in the levels of TNF-a, MIP-1a, MIP-1b and IL-15. We found that wildtype RAW cells cultured in the presence the STING inhibitor, RU.521, or cGAS-deficient RAW cells stimulated with ONP-302 failed to upregulate expression of TNF-a, MIP-1a, MIP-1b, and IL-15 compared to controls (FIG. 8A). In addition, we tested GM-CSF driven bone marrow-derived myeloid cells from wildtype vs. STING^{-/-} mice. Bone marrow cells from both strains were cultured in the presence of 25 ng/mL GM-CSF for 7 days at which time the cell phenotypes were analyzed by flow. The remaining cells were cultured in the presence of LPS (1 mg/mL) or ONP-302 (2.5 mg/mL) for 24 hours and the levels of secreted cytokines/chemokines analyzed. Lipopolysaccharide (LPS)-induced the secretion of TNF-a, MIP-1a, MIP-1b, and IL-15 by both wildtype and STING^{-/-} myeloid cells, while cytokine secretion in response to ONP-302 was lost in the absence of STING expression (FIG. 8B).

Taken together, these data show that phagocytosis of ONP-302 by myeloid cells results in activation of the cGAS/STING pathway leading to secretion of TNF-a, MIP-1a, MIP-1b, and IL-15.

### Example 7: Method of Manufacturing Negatively Charged ONP-302 Nanoparticles

One method of manufacturing negatively charged surface functionalized PLGA particles (ONP-302) is using a double-emulsion process. The manufacturing process uses PLGA, polyvinyl alcohol (PVA), polyacrylic acid (PAA), and Ethyl Acetate (EtOAc).

PLGA used for manufacturing is a 50:50 co-polymer of lactic acid and glycolic acid (±5 mole %) with a molecular weight of between 10-75 kDa, a result of ring-opening polymerization from lactide and glycolide monomers.

A double emulsion is initiated by first generating a water-in-oil primary emulsion, in which PLGA solution (5% in ethyl acetate) is rapidly mixed with sterile water for injection (WFI). The primary emulsion is rapidly mixed with a surfactant and stabilizer solution (4% PVA and PAA (Sigma Aldrich, 100 kDa, 35% wt.)) in ethyl acetate to form an oil-in-water secondary emulsion such that the ratios of PAA/PVA/EtOAc in the emulsion are approximately 1:4:12.5% wt. The composition of the PVA/PAA/Ethyl acetate blend is maintained at a pH below 4.0. Mixing of the primary and secondary emulsions is performed by homogenization.

The nanoparticles formed in the secondary emulsion are then hardened by removing the solvent through evaporation under vacuum (25-35 mBar for at least 4 hours) followed by passive evaporation for 12-72 hours. Hardened nanoparticles are passed through a 20 μm filter prior to initiating tangential flow filtration (TFF). The nanoparticles are then filtered, washed, and concentrated via tangential flow filtration (TFF) to remove residual surfactants and solvent. Concentrated nanoparticles are sampled for in-process analysis to confirm size of ≥300 nm and a zeta potential of <−20 mV.

Cryoprotectant and buffering excipients sucrose, mannitol, and sodium citrate are then added to the concentrated nanoparticles. Nanoparticles are then filled into vials and lyophilized. The physicochemical properties of the final ONP-302 nanoparticle formulation are summarized in Table 2 below:

TABLE 2
ONP-302 Physicochemical Properties

| ATTRIBUTE | RESULT | |
|---|---|---|
| Size by Dynamic Light Scattering | 550 | nm |
| Size by Laser Diffraction | 510 | nm |
| Size distribution by Single Particle Optical | 520/560/630 nm | |
| Sensing (SPOS) | (d₁₀/d₅₀/d₉₀) | |
| Zeta potential by Dynamic Light Scattering | −54 | mV |
| pH | 5.7 | |
| Moisture content | 0.7% | |
| Residual ethyl acetate | 2,120 | ppm |

| | |
|---|---|
| PVA and PAA content by NMR | PVA: 0.07% wt. |
| | PAA: 0.54% wt. |
| Sodium citrate content by NMR | 1.99% |
| Sucrose and Mannitol content by RI-HPLC | Sucrose: 70.3 mg/vial |
| | Mannitol: 98.1 mg/vial |
| Nanoparticle content by Flow Cytometry | 1.1 × 1011 count/vial |

What is claimed is:

1. A method of treating cancer in a subject in need thereof, comprising administering negatively charged particles at a dose ranging from about 0.1 mg/kg to about 15 mg/kg, wherein the negatively charged particles comprise:
a) a zeta potential ranging from about −10 mV to −100 mV; and
b) an average diameter ranging from about 100 nm to about 1500 nm,
wherein the negatively charged particles are free from a therapeutic agent, thereby treating the cancer.

2. The method of claim 1, wherein the negatively charged particles are free from a small molecule.

3. The method of claim 1, wherein the negatively charged particles are free from a peptide.

4. The method of any one of the preceding claims, wherein the negatively charged particles are administered by infusion.

5. The method of any one of the preceding claims, wherein the negatively charged particles are present in a pharmaceutical composition at a concentration ranging from about 0.05 mg/mL to about 50 mg/mL.

6. The method of any one of the preceding claims, wherein the negatively charged particles are administered at a dose ranging from about 1.5 mg/kg to about 2.5 mg/kg, 3.5 mg/kg to about 4.5 mg/kg, or 7.5 mg/kg to about 8.5 mg/kg.

7. The method of any one of the preceding claims, wherein the negatively charged particles are administered at a dose ranging about 10 mg to about 1000 mg.

8. The method of any one of the preceding claims, wherein the negatively charged particles are administered at a dose of about 10 mg, 25 mg, 50 mg, 75 mg, 100 mg, 125 mg, 150 mg, 175 mg, 200 mg, 225 mg, 250 mg, 275 mg, 300 mg, 325 mg, 350 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg, 800 mg, 850 mg, 875 mg, 900 mg, 950 mg, 975 mg, or 1000 mg.

9. The method of any one of claims 1-8, wherein the negatively charged particles are administered in multiple doses.

10. The method of any one of claims 1-8, wherein the negatively charged particles are administered in a single dose.

11. The method of any one of claims 1-8, wherein the negatively charged particles are administered once daily, twice daily, three times per day, seven times per week, six times per week, five times per week, four times per week, three times per week, twice weekly, once weekly, once every two weeks, once every three weeks, once every 4 weeks, once every two months, once every three months, once every 6 months, or once per year.

12. The method of any one of the preceding claims, wherein the negatively charged particles are administered in treatment cycles.

13. The method of claim 12, wherein the each treatment cycle is a week, a month, or a year in duration.

14. The method of claim 13, wherein the treatment cycle is a week in duration, and the negatively charged particles are administered once per week.

15. The method of claim 13 or 14, wherein the treatment cycle is a week in duration, and the negatively charged particles are administered over four treatment cycles.

16. The method of claims 12-15, wherein the negatively charged particles are administered in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, or 52 treatment cycles.

17. The method of claim 1, wherein the negatively charged particles are administered intravenously, subcutaneously, intramuscularly, intraperitoneally, intranasally, or orally.

18. The method of any one of the preceding claims, wherein the negatively charged particles are administered alone or in combination with one or more additional cancer therapeutics.

19. The method of claim 18, wherein the negatively charged particles are administered prior to, concomitantly, or after the administration of the one or more additional cancer therapeutics.

20. The method of any one of the preceding claims, wherein the negatively charged particles are administered prior to or after surgical resection of a tumor.

21. The method of any one of the preceding claims, wherein administering negatively charged particles alone or in combination with one or more additional cancer therapeutics reduces one or more symptoms of cancer.

22. The method of claim 21, wherein the administering of the negatively charged particles reduces tumor size and/or tumor burden in the subject in need thereof.

23. The method of claim 22, wherein the tumor size and/or tumor burden in the subject is reduced by about 1%, about 2%, about 3%, about 4%, about 5%, about 6%, about 7%, about 8%, about 9%, about 10%, about 20%, about 30%, about 40%, about 50%, about 60%, about 70%, about 80%, about 90% or about 100%.

24. The method of claim 22, wherein change in tumor size and/or tumor burden is determined using Response Evaluation Criteria in Solid Tumours (RECIST).

25. The method of claim 21, wherein administering the negatively charged particles reduces the number of circulating tumor cells (CTCs) in the subject in need thereof.

26. The method of claim 25, wherein the CTCs are reduced by at least about 1%, about 2%, about 3%, about 4%, about 5%, about 6%, about 7%, about 8%, about 9%, about 10%, about 20%, about 30%, about 40%, about 50%, about 60%, about 70%, about 80%, about 90% or about 100% compared to the CTCs prior to treatment.

27. The method of claim 21, wherein the administering of the negatively charged particles reduces the number of CTC clusters in the subject in need thereof.

28. The method of claim 27, wherein the number of CTC clusters is reduced by about 1%, about 2%, about 3%, about 4%, about 5%, about 6%, about 7%, about 8%, about 9%, about 10%, about 20%, about 30%, about 40%, about 50%, about 60%, about 70%, about 80%, about 90% or about 100% compared to the CTC clusters prior to treatment.

29. The method of claim 21, wherein the administering of the negatively charged particles reduces the number of CTC-leukocyte clusters in the subject in need thereof.

30. The method of claim 29, wherein the number of CTC-leukocyte clusters is reduced by about 1%, about 2%, about 3%, about 4%, about 5%, about 6%, about 7%, about 8%, about 9%, about 10%, about 20%, about 30%, about 40%, about 50%, about 60%, about 70%, about 80%, about 90% or about 100% compared to the number of CTC-leukocyte clusters prior to treatment.

31. The method of claim 29 or 30, wherein the leukocyte is a myeloid-derived cell.

32. The method of any one of claims 29-31, wherein the myeloid derived cell is a monocyte, macrophage, neutrophil, or dendritic cell.

33. The method of any one of the preceding claims, wherein the administering extends survival of the subject in need thereof.

34. The method of claim 33, wherein the survival is extended by about 1 year to about 50 years.

35. The methods of any one of the preceding claims, wherein the administering of negatively charged particles induces an anti-cancer immune response.

36. The method of claim 35, wherein the anti-cancer immune response comprises an increase in the number of anti-cancer T cells, NK cells, B cells, and/or myeloid derived cells.

37. The method of claim 35, wherein the anti-cancer immune response comprises an increase in the number of activated anti-tumor T cells, NK cells, B cells, and/or myeloid derived cells.

38. The method of claim 36 or 37, wherein the myeloid derived cells are monocytes, macrophages, neutrophils, or dendritic cells.

39. The method of claim 35, wherein the anti-cancer immune response comprises an increase in the concentration of one or more anti-cancer cytokines and/or chemokines.

40. The method of claim 39, wherein the one or more anti-cancer cytokines and/or chemokines are soluble IL-15, IL-17, TNF-α, IFN-γ, MIP-1α, MIP-1β, RANTES, TGF-β1, TGF-β2, VEGF, or M-CSF.

41. The method of claim 35, wherein the anti-cancer immune response comprises an increase in anti-tumor gene expression levels in T cells, NK cells, B cells, and/or myeloid derived cells.

42. The method of claim 41, wherein the myeloid derived cells are monocytes, macrophages, neutrophils, or dendritic cells.

43. The method of claim 35, wherein the anti-cancer immune response is detected in one or more of: a tumor, blood, spleen, or lymph nodes.

44. The method of claim 35, wherein the anti-cancer immune response is detected in one or more sites of metastasis.

45. The method of claim 35, wherein the anti-cancer immune response is measured in one or more biological samples comprising whole-blood, peripheral blood, peripheral blood mononuclear cells (PBMCs), serum, plasma, urine, cerebrospinal fluid (CSF), stool, a tissue biopsy, or a bone-marrow biopsy.

46. The method of any one of the preceding claims, wherein the administration of the negatively charged particles reduces the levels of neutrophil extracellular traps (NETs).

47. The method of any one of the preceding claims, wherein the administration of the negatively charged particles reduces the neutrophil-to-lymphocyte ratio (NLR).

48. The method of any one of the preceding claims, wherein the cancer is metastatic cancer.

49. The method of any one of the preceding claims, wherein the cancer is a solid cancer.

50. The method of any one of the preceding claims, wherein the cancer is brain cancer, skin cancer, eye cancer, breast cancer, prostate cancer, lung cancer, esophageal cancer, head and neck cancer, cervical cancer, liver cancer, colon cancer, bone cancer, uterine cancer, ovarian cancer, bladder cancer, stomach cancer, oral cancer, thyroid cancer, kidney cancer, or testicular cancer.

51. A method for preparing a composition comprising negatively charged particles, the method comprising:
(a) generating a primary emulsion by mixing an aqueous solution with an oil phase including a polymer;
(b) mixing the primary emulsion with a solution including one or more surfactants and/or stabilizers to form a secondary emulsion;
(c) removing the solvent to form crude negatively charged particles; and
(d) filtering the crude particles to form a composition comprising negatively charged particles

52. The method of claim 51, wherein the solution of step (a) includes a solvent.

53. The method of claim 51, wherein the solution of step (b) includes a solvent.

54. The method of any one of claims 51-53, wherein the solvent is an organic solvent

55. The method of any one of claims 51-54, wherein the solutions of step (a) and step (b) include the same solvent.

56. The method of any one of claims 51-54, wherein the solutions of step (a) and step (b) include different solvents.

57. The method of claim 54, wherein the organic solvent is dichloromethane, acetone, ethanol, methylene chloride, dimethyl sulfoxide (DMSO), ethyl acetate, dimethylformamide, tetrahydrofuran, chloroform, and acetic acid.

58. The method of any one of claims 51-57, wherein the emulsion resulting from step (a) is a water-in-oil emulsion.

59. The method of any one of claims 51-58, wherein the emulsion resulting from step (b) is an oil-in-water emulsion.

60. The method of any one of claims 51-59, wherein the polymer of step (a) is a biodegradable polymer.

61. The method of claim 60, wherein the biodegradable polymer is polyglycolic acid (PGA), polylactic acid (PLA), polysebacic acid (PSA), poly(lactic-co-glycolic) (PLGA), poly(lactic-co-sebacic) acid (PLSA), poly(glycolic-co-sebacic) acid (PGSA), polypropylene sulfide, poly(caprolactone), chitosan, a polysaccharide, or a lipid.

62. The method of any one of claims 51-61, wherein the surfactant or stabilizer of step (b) is anionic.

63. The method of claim 62, wherein the surfactant and/or stabilizer is a poloxamer, a polyamine, PEG, Tween-80, gelatin, dextran, pluronic L-63, pluronic F-68, pluronic 188, pluronic F-127, PVA, PAA, methylcellulose, lecithin, DMAB, PEMA, vitamin E TPGS (D-a-tocopheryl polyethylene glycol 1000 succinate), hyaluronic acid, poly amino acids (e.g polymers of lysine, arginine, aspartic acid, glutamic acid, serine, threonine, tyrosine and cysteine, or their enantiomers), methylcellulose, hydroxyethylcellulose, hydroxyprolylcellulose, hydroxypropylmethylcellulose, gelatin, sodium cholate, a carbomer, or a sulfate polymer.

64. The method of any one of claims 51-63, wherein the primary emulsion of step (a) is obtained by homogenization.

65. The method of any one of claims 51-63, wherein the primary emulsion of step (a) is obtained by sonication.

66. The method of any one of claims 51-65, wherein the secondary emulsion of step (b) is obtained by homogenization.

67. The method of any one of claims 51-65, wherein the secondary emulsion of step (b) is obtained by sonication.

68. The method of any one of claims 51-67, wherein the pH of the secondary emulsion of step (b) is less than pH 4.

69. The method of claim 64 or 66, wherein homogenization is performed for 5, 10, 15, 20, 25, 30, 30, 40, 45, 60, 90, 120, 150, 180, 210, 240, 270, 300, 330, 360, 390, 420, 450, 480, 510, 540, 570, or 600 seconds.

70. The method of claim 65 or 67, wherein sonication is performed for 5, 10, 15, 20, 25, 30, 30, 40, 45, 60, 90, 120, 150, 180, 210, 240, 270, 300, 330, 360, 390, 420, 450, 480, 510, 540, 570, or 600 seconds.

71. The method of any one of claims 51-70, wherein the removing in step (c) is performed by evaporation of the solvent.

72. The method of claim 71, wherein evaporation is active evaporation or passive evaporation.

73. The method of claim 72, wherein active evaporation is vacuum-driven evaporation.

74. The method of claim 73, wherein the vacuum-driven evaporation is performed under high pressure or low pressure.

75. The method of claim 72, wherein passive evaporation is performed by stirring.

76. The method of claim 75, wherein evaporation is performed for 0.25, 0.5, 1, 2, 3, 4, 5, 6, 7, 8, 9, 10, 11, 12, 13, 14, 15, 16, 17, 18, 19, 20, 21, 22, 23, 24, 48, 72, or 96 hours.

77. The method of any one of claims 51-76, wherein filtering, washing, and concentrating the nanoparticles in step (d) is performed by filtration, gel filtration, membrane filtration, dialysis, centrifugation, chromatography, density gradient centrifugation, or combinations thereof.

78. The method of any one of claims 51-77, wherein the particles have a negative zeta potential.

79. The method of claim 78, wherein the zeta potential of the particles is about −1 mV to about −100 mV.

80. The method of claim 79, wherein the zeta potential of the particles is about −30 mV to about −80 mV.

81. The method of any one of claims 51-80, wherein the particles have an average diameter of about 0.3 μm to about 3 μm.

82. The method of claim 81, wherein the particles have an average diameter of about 0.3 μm to about 1 μm.

83. The method of claim 82, wherein the particles have an average diameter of about 0.4 μm to about 1 μm.

84. The method of any one of claims 51-83, wherein at least 90% of the particles have an average diameter of about 0.3 μm to about 3 μm.

85. The method of claim 84, wherein at least 90% of the particles have an average diameter of about 0.3 μm to about 1 μm.

86. The method of claim 85, wherein at least 90% of the particles have an average diameter of about 0.4 μm to about 1 μm.

87. The method of any one of claims 51-86, wherein at least 50% of the particles have an average diameter of about 0.3 μm to about 3 μm.

88. The method of claim 87, wherein at least 50% of the particles have an average diameter of about 0.3 μm to 1 μm.

89. The method of claim 88, wherein at least 50% of the particles have an average diameter of about 0.4 μm to 1 μm.

90. The method of any one of claims 51-89, wherein at least 10% of the particles have an average diameter of about 0.3 μm to 3 μm.

91. The method of claim 90, wherein at least 10% of the particles have an average diameter of about 0.3 μm to 1 μm.

92. The method of any one of the preceding claims, wherein the manufacturing batch size is about 0.01 g to 100 kg.

93. The method of claim 92, wherein the manufacturing batch size is 0.01 g, 0.1 g, 10 g, 20 g, 40 g, 60 g, 80 g, 100 g, 160 g, 240 g, 320 g, 400 g, 480 g, 560 g, 640 g, 720 g, 800 g, 1000 g, 5 kg, 10 kg, 50 kg, or 100 kg.

94. A composition comprising particles made by the method of any one of claims 51-93.

95. The composition of claim 94, further comprising a pharmaceutically acceptable excipient.

96. The composition of claim 95, wherein the excipient comprise sucrose, mannitol, and sodium citrate.

97. A pharmaceutical composition comprising negatively charged particles, sucrose, mannitol, and sodium citrate.

98. A method of treating a subject having cancer comprising administering the composition of any one of claims 94-97.

99. The method of claim 1, wherein the negative charged particles are administered at a dose of about 2 mg/kg.

100. The method of claim 1, wherein the negative charged particles are administered at a dose of about 4 mg/kg.

101. The method of claim 1, wherein the negative charged particles are administered at a dose of about 10 mg/kg.

102. A method of treating a solid tumor in a subject in need thereof, comprising administering negatively charged particles at a dose ranging from about 0.4 mg/kg to about 10 mg/kg, wherein the negatively charged particles have:
a) a zeta potential of about −10 mV to about −100 mV; and
b) an average diameter of about 100 nm to about 1500 nm,
wherein the negatively charged particles are free from a therapeutic agent.

103. The method of claim 102, wherein the negative charged particles are administered at a dose of about 2 mg/kg.

104. The method of claim 102, wherein the negative charged particles are administered at a dose of about 4 mg/kg.

105. The method of any one of claims 102-104, wherein the cancer is selected from the group consisting of: brain cancer, skin cancer, eye cancer, breast cancer, prostate cancer, lung cancer, esophageal cancer, head and neck cancer, cervical cancer, liver cancer, colon cancer, bone cancer, uterine cancer, ovarian cancer, bladder cancer, stomach cancer, oral cancer, thyroid cancer, kidney cancer, or testicular cancer.

106. The method of any one of the preceding claims, wherein the negatively charged particles have a D90 of about 600 nm to about 700 nm.

107. The method of any one of the preceding claims, wherein the negatively charged particles have a D50 of about 550 nm to about 600 nm.

108. The method of any one of the preceding claims, wherein the negatively charged particles have a D10 of about 500 nm to about 550 nm.

109. The method of claim 55, wherein the crude particles in step (d) are filtered through a filter with a pore size of about 10 μm to about 20 μm.

110. The method of claim 55, wherein the step (d) further comprises washing of the filtered crude particles.

111. The method of claim 55, wherein the step (d) further comprises concentrating of the filtered crude particles.

112. The method of claim 51, wherein the composition comprises less than 0.5% wt. of the one or more surfactants and less than 1% wt. of the one or more stabilizers.
